(19) United States
(12) Patent Application Publication
(10) Pub. No.: US 2003/0166201 A1
(43) Pub. Date: Sep. 4, 2003

(54) Selection systems for genetically modified cells

(76) Inventor: Michael C. Jensen, Pasadena, CA (US)

Correspondence Address: HELLER EHRMAN WHITE & MCAULIFFE LLP, 4350 LA JOLLA VILLAGE DRIVE, 7TH FLOOR, SAN DIEGO, CA, 92122-1246, US

(21) Appl. No.: 09/846,637

(22) Filed: Apr. 30, 2001

(51) Int. Cl. A61K 48/00; C12N 9/06; C07H 21/04; C12P 21/02; C12N 5/06

(52) U.S. Cl. 435/191; 435/69.1; 435/320.1; 435/325; 536/23.2; 514/44

(57) ABSTRACT

Compositions and methods for use in generating and selecting genetically modified cells are provided. The compositions include selectable markers and selection systems based thereon. Also provided are methods for the introduction and expression of heterologous nucleic acids in host animals that use the compositions and methods for generating and selecting genetically modified cells.

### FIELD OF THE INVENTION

The present invention relates to compositions and methods for use in generating and selecting genetically modified cells. The invention further relates to methods for the introduction and expression of heterologous nucleic acids in host animals that use the compositions and methods for generating and selecting genetically modified cells.

### BACKGROUND OF THE INVENTION

Incorporation of heterologous DNA into host cells for the expression of the DNA therein is a widely used process in connection with, for example, recombinant protein production, the generation of transgenic animals and gene therapy. Typically, it is preferred that the cells into which the heterologous nucleic acid is introduced stably express the heterologous nucleic acid on a long-term basis over many generations of the transformed cell population. Stable transformation of host cells with heterologous nucleic acid involves the use of selection systems that permit the identification of cells that have stably integrated the heterologous nucleic acid into the host chromosomes.

Generally, selection systems for identification of stable transformants are based in the acquisition of a resistance phenotype by these cells through the integration of a selectable marker gene into the host cell genome. The marker gene is one that encodes a product that confers resistance to an agent that is toxic to the host cell. If the marker is a recessive selection marker, such as thymidine kinase (TK) or hypoxanthine-guanine phosphoribosyl transferase (HPRT), the host cell line must be a recessive mutant cell line (e.g., tk⁻ or hprt⁻) that is deficient in the marker enzyme. If the marker is a dominant selection marker, e.g., neomycin phosphotransferase or hygromycin phosphotransferase, a broader range of host cells is available since most cells are sensitive to the cytotoxic effects of selection agents, such as neomycin and hygromycin, that are used in these types of selection systems.

In either selection scheme, the marker gene is introduced into host cells along with heterologous nucleic acid of interest, and the transfected cells are cultured in medium containing an appropriate cytotoxic agent. Only those cells that have taken up and stably maintain and express the heterologous nucleic acid, including the selectable marker gene, will survive growth in the presence of the agent. As a result, transformants that have incorporated the heterologous nucleic acid are selected.

The selection of genetically modified cells is a critical process in ex vivo and in vivo gene therapy applications. In ex vivo gene therapy methods, it is of paramount importance to identify and isolate stably transformed host cells in order to ensure that only those cells that contain therapeutic heterologous nucleic acid integrated into the cell genome are introduced into subjects undergoing the therapy. Due to the limited survival time of the transformed cells in the in vivo environment and the finite duration of therapeutic heterologous nucleic acid expression in the transformed cells, successful ex vivo gene therapy requires from the outset that a homogeneous population of transformed cells capable of sustained, high-level expression of therapeutic genes be introduced into the recipient subject.

In comparison to ex vivo gene therapy methods, there is much less control over and manipulation of the process of gene transfer into target cells in in vivo gene therapy methods. The efficacy of in vivo gene therapy methods depends in large part on the ability to retain and propagate cells of an organism undergoing therapy that have integrated, in vivo, the therapeutic heterologous nucleic acid into the cellular genome and to maximize expression of the nucleic acid in those transformed cells. Therefore, an effective selection means could be a major factor in the success of in vivo gene therapy methods.

A disadvantage of selection systems based on recessive markers is the limited availability of host cell lines. In these systems, it is necessary to utilize one of the currently available recessive mutant cell lines as a host cell line, or to generate and characterize a recessive mutant derivative cell line of a wild-type cell line. This requirement imposes limitations on ex vivo gene therapy methods, and makes recessive marker-based selection systems unusable for in vivo gene therapy methods. One possible advantage of recessive marker-based selection systems, however, is the potential for maintaining selective conditions in vivo through administration of a selection agent that is not toxic to the organism undergoing the gene therapy.

The most common dominant marker selection strategies involve the expression of prokaryotic marker genes, such as the genes encoding neomycin phosphotransferase and hygromycin phosphotransferase, for selection. One disadvantage of the use of these systems in connection with gene therapy methods is associated with the immunogenicity of the bacterial selection proteins when selected transformant cells are introduced into immunocompetent hosts. Because the selected cells produce immunogenic proteins foreign to eukaryotes, the persistence of the selected cells in an immunocompetent eukaryotic host is limited due to elimination of the cells by the host's immune system.

Non-immunogenic “markers” for use in gene therapy have been suggested (see, e.g., WO98/30709). The suggested markers (e.g., alkaline phosphatase, α-galactosidase, β-glucosidase, β-glucuronidase, carboxypeptidase A, cytochrome P450, γ-glutamyl transferase, azoreductase, DT diaphorase, nitroreductase, glucose oxidase and xanthine oxidase), although referred to as “selectable,” are described as being included in gene delivery vehicles to allow for simpler preparation, manufacturing, characterization or testing of gene delivery vehicles. Thus, these “markers” are not designed for or even capable of conferring a phenotype, such as a drug-resistance phenotype, on cells containing them that enables the selective proliferation of the marker-containing cells over cells that do not contain the marker. Instead, the suggested “markers” at most provide only a “tag” for identifying cells that express the marker. It is therefore still necessary to isolate the marker-containing cells from cells that do not contain the marker. Furthermore, because these markers provide no means for selectively reducing proliferation of cells that do not express the marker while promoting the proliferation of marker-containing cells, they are not useful in in vivo gene therapy methods.

Another disadvantage of most truly selectable dominant marker-based selection systems is the inability to maintain selective conditions in vivo due to the general cytotoxicity of the selection agents used in these systems. This is clearly a significant shortcoming of these systems, since continued selective pressure on transformed cells in vivo may be associated with increased levels of heterologous gene expression in the cells [see, e.g., Licht et al. (1997) Stem Cells 15:104-111]. Therefore, it is desirable to be able to maintain a selective environment in vivo during gene therapy.

There is a need, therefore, for selection systems that provide for the selective survival of transformed, therapeutic gene-containing cells but that are unhampered by the generation of an immune response directed against the selected cells and that operate without undesired harmful effects in a subject undergoing genetic therapy.

### SUMMARY OF THE INVENTION

Compositions and methods for use in generating and selecting genetically modified cells are provided herein. The compositions and methods may be used for in vitro or in vivo generation and selection of genetically modified cells. Also provided are methods for the introduction and expression of heterologous nucleic acids in host animals that use the compositions and methods for generating and selecting genetically modified cells. Gene therapy methods using the compositions and methods for generating and selecting genetically modified cells are also provided.

The compositions provided herein include selectable markers and selection systems based thereon. The selection systems disclosed herein have a variety of uses, including, but not limited to, identifying, isolating, differentially affecting and/or providing a selective advantage for proliferation and/or viability of certain cells that have been genetically modified.

Particular selection systems are based in the pairing of a selectable marker with selection agents that provide conditions under which cells containing the selectable marker will exhibit greater proliferation and/or viability than at least substantially identical cells that do not contain the selectable marker. Selectable markers that may be used in such selection systems include inhibitor-resistant or altered enzymes involved in the synthesis of nucleotides in cells. The activity of such enzymes is sufficient to effectively participate in the particular biosynthetic reactions in which they are involved and thereby provide for nucleotide levels required to sustain cells and/or cell growth. Selection agents used in such selection systems include compositions and/or conditions which inhibit sensitive (i.e., non-resistant or unaltered) forms of the enzymes in cells such that at least certain cells exposed to the inhibitor under certain conditions exhibit reduced proliferation and/or viability relative to the amount of proliferation and/or viability of the cells in the absence of the inhibitor.

Exemplary selectable markers which may be used in selection systems as described herein include inhibitor-resistant or altered forms of enzymes of the purine and pyrimidine biosynthesis pathways. Such enzymes include, but are not limited to, inosine monophosphate dehydrogenase (IMPDH), dihydroorotate dehydrogenase (DHODH), ribose phosphate pyrophosphokinase, amidophosphoribosyltransferase, glycinamide ribonucleotide (GAR) synthetase, GAR transformylase, formylglycinamidine ribonucleotide (FGAM) synthetase, aminoimidazole ribonucleotide (AIR) synthetase, AIR carboxylase, aminoimidazolesuccinocarboxamide ribonucleotide (SAICAR) synthetase, adenylosuccinate synthase, adenylosuccinate lyase, aminoimidazolecarboxamide ribonucleotide (AICAR) transformylase, IMP cyclohydrolase, GMP synthase, carbamoyl phosphate synthetase II, aspartate transcarbamylase, dihydroorotase, orotate phosphoribosyl transferase, OMP decarboxylase, CTP synthase, thymidylate synthase, and ribonucleotide reductase. Inhibitors of these enzymes, which may be useful as selection agents in systems using inhibitor-resistant forms of these enzymes as selectable markers, are known and can be identified by those of skill in the art. Included among such inhibitors are the following and derivatives, analogs and metabolites thereof: mycophenolic acid, tiazofurin, ribavirin, EICAR, mizoribine, pyridazines, cinchoninic acid derivatives, naphthoquinone derivatives, isoxazole derivatives, acivicin, 6-mercaptopurine (an inhibitor of adenylosuccinate synthase and adenylosuccinate lyase) and folate anti-metabolites such as 5,10-dideaza-5,6,7,8-tetrahydrofolic acid (DDATHF, lometrexol; inhibitors of the folate requiring enzymes GAR transformylase and AICAR transformylase).

In a particular embodiment of the selection systems provided herein, an inhibitor-resistant or altered enzyme of a purine biosynthesis pathway is used as a selectable marker. The enzyme may be a rate-limiting enzyme of de novo purine nucleotide biosynthesis. For example, an inhibitor-resistant or altered inosine monophosphate dehydrogenase (IMPDH) enzyme (particularly a mammalian, such as rodent and human, IMPDH), which serves as a selectable marker, is used to confer on cells in which it is expressed resistance to an inhibitor of IMPDH (i.e., a selection agent). If the selectable marker is an altered IMPDH, the alteration in the IMPDH is such that the enzyme is no longer effectively inhibited by the selection agent.

Inhibitor-resistant IMPDH enzymes are IMPDH enzymes that are resistant to one or more conditions or agents that are effective inhibitors of other IMPDH enzymes and thus include altered IMPDH enzymes. Inhibitor-resistant IMPDH enzymes that are not altered include, for example, some microbial IMPDH enzymes which are resistant to agents that inhibit mammalian IMPDH enzymes. Tritrichomonas foetus IMPDH is poorly inhibited by mycophenolic acid (MPA), which is an effective inhibitor of most mammalian IMPDH enzymes. T. foetus IMPDH enzymes include those with substantially the same amino acid sequence set forth in SEQ. ID. NO. 14. Nucleic acid encoding the this enzyme includes molecules with the sequence of nucleotides set forth in SEQ. ID. NO. 13.

Altered IMPDH enzymes include altered IMPDH type I and type II enzymes having alterations relative to the amino acid sequences set forth in SEQ. ID. NOS. 2, 18 and 30 that impart resistance on the enzyme to an IMPDH inhibitor. Included among such altered IMPDH enzymes are those that contain one or more alterations in the sequence of amino acids 330-451, 330-441, 330-400, 330-355 or 333-351 of SEQ. ID. NOS. 2, 18 and 30. Particular alterations of the amino acid sequence set forth in SEQ. ID. NO. 2 include one or more of the following: sequences in which amino acid 333 is other than threonine, amino acid 351 is other than serine, amino acid 277 is other than glutamine, amino acid 462 is other than alanine, amino acid 456 is other than phenylalanine and amino acid 470 is other than aspartic acid. Particular alterations of the amino acid sequence set forth in SEQ. ID. NO. 30 include sequences in which amino acid 333 is other than threonine and/or amino acid 351 is other than serine. One such altered IMPDH is a mutant mouse IMPDH type II (see SEQ ID NO. 32). A nucleotide sequence encoding this altered mouse type II IMPDH is provided in SEQ ID NO. 31.

Inhibitor-resistant enzymes may be altered human IMPDH enzymes. Provided herein are isolated altered human IMPDH enzymes that contain one or more alterations in:

(a) the sequence of amino acids from a position corresponding to amino acid 330 to a position corresponding to amino acid 451 of SEQ. ID. NO. 2;

(b) the sequence of amino acids from a position corresponding to amino acid 330 to a position corresponding to amino acid 441 of SEQ. ID. NO. 2;

(c) the sequence of amino acids from a position corresponding to amino acid 330 to a position corresponding to amino acid 400 of SEQ. ID. NO. 2;

(d) the sequence of amino acids from a position corresponding to amino acid 330 to a position corresponding to amino acid 355 of SEQ. ID. NO. 2; or

(e) the sequence of amino acids from a position corresponding to amino acid 333 to a position corresponding to amino acid 351 of SEQ. ID. NO. 2; whereby the altered IMPDH is resistant to a purine biosynthesis inhibitor. In a particular embodiment, the altered human IMPDH enzyme is resistant to mycophenolic acid or a derivative, analog or metabolite thereof. Included among the isolated altered human IMPDH enzymes provided herein are those containing one or more alterations in the sequence of amino acids from a position corresponding to amino acid 333 to a position corresponding to amino acid 351 of SEQ. ID. NO. 2. Examples of such enzymes are those containing an amino acid other than threonine at a position corresponding to amino acid 333 of SEQ. ID. NO. 2 or an amino acid other than serine at a position corresponding to amino acid 351 of SEQ. ID. NO. 2 or an amino acid other than threonine at a position corresponding to amino acid 333 of SEQ. ID. NO. 2 and an amino acid other than serine at a position corresponding to amino acid 351 of SEQ. ID. NO. 2. Particular enzymes are those wherein the amino acid other than threonine at a position corresponding to amino acid 333 is isoleucine or a conservative substitution thereof and the amino acid other than serine at a position corresponding to amino acid 351 is tyrosine or a conservative substitution thereof. One such enzyme provided herein has substantially the same sequence of amino acids as set forth in SEQ ID NO. 4 or the sequence of amino acids as set forth in SEQ. ID. NO. 4 except that amino acids 190 and 191 are alanine and glycine, respectively.

Isolated nucleic acid is also provided that encodes altered human IMPDH enzymes that contain one or more alterations in:

(a) the sequence of amino acids from a position corresponding to amino acid 330 to a position corresponding to amino acid 451 of SEQ. ID. NO. 2;

(b) the sequence of amino acids from a position corresponding to amino acid 330 to a position corresponding to amino acid 441 of SEQ. ID. NO. 2;

(c) the sequence of amino acids from a position corresponding to amino acid 330 to a position corresponding to amino acid 400 of SEQ. ID. NO. 2;

(d) the sequence of amino acids from a position corresponding to amino acid 330 to a position corresponding to amino acid 355 of SEQ. ID. NO. 2; or

(e) the sequence of amino acids from a position corresponding to amino acid 333 to a position corresponding to amino acid 351 of SEQ. ID. NO. 2; whereby the altered IMPDH is resistant to a purine biosynthesis inhibitor. In a particular embodiment, the altered human IMPDH enzyme encoded by the isolated nucleic acid is resistant to mycophenolic acid or a derivative, analog or metabolite thereof. Included among the isolated nucleic acids encoding altered human IMPDH enzymes provided herein are those encoding enzymes containing one or more alterations in the sequence of amino acids from a position corresponding to amino acid 333 to a position corresponding to amino acid 351 of SEQ. ID. NO. 2. Examples of such enzymes are those containing an amino acid other than threonine at a position corresponding to amino acid 333 of SEQ. ID. NO. 2 or an amino acid other than serine at a position corresponding to amino acid 351 of SEQ. ID. NO. 2 or an amino acid other than threonine at a position corresponding to amino acid 333 of SEQ. ID. NO. 2 and an amino acid other than serine at a position corresponding to amino acid 351 of SEQ. ID. NO. 2. Particular enzymes encoded by the isolated nucleic acids are those wherein the amino acid other than threonine at a position corresponding to amino acid 333 is isoleucine or a conservative substitution thereof and the amino acid other than serine at a position corresponding to amino acid 351 is tyrosine or a conservative substitution thereof. For example, nucleic acid encoding an altered human IMPDH enzyme with an amino acid sequence set forth in SEQ. ID. NO. 4, or as in SEQ. ID. NO. 4 except that amino acids 190 and 191 are alanine and glycine, respectively, has been generated by site-directed mutagenesis. A nucleotide sequence encoding this altered human IMPDH includes nucleotides 48 to 1589 of SEQ. ID. NO. 3, or nucleotides 48 to 1589 of SEQ. ID. NO. 3 except that the sequence of nucleotides 614-619 is TGCAGG instead of CCGCAG. Also provided herein are vectors containing these nucleic acids encoding altered human IMPDH enzymes. Further provided are cells containing heterologous nucleic acids which include the nucleic acids encoding altered human IMPDH enzymes that contain one or more alterations as described above. In particular embodiments, the cells are lymphocytes, including, but not limited to, T- and B-lymphocytes.

Also provided herein are non-cancerous human lymphocytes, containing a heterologous altered inosine monophosphate dehydrogenase (IMPDH) that is resistant to a purine biosynthesis inhibitor and/or heterologous nucleic acid encoding an altered inosine monophosphate dehydrogenase (IMPDH) that is resistant to a purine biosynthesis inhibitor.

Other inhibitor-resistant human IMPDH enzymes include altered enzymes having substantially the same amino acid sequences set forth in SEQ. ID. NOS. 6, 8, 10 and 12. Nucleotide sequences encoding enzymes having substantially the same amino acid sequences set forth in SEQ. ID. NOS. 6, 8, 10 and 12 include those set forth in SEQ. ID. NOS. 5, 7, 9 and 11, respectively.

In another embodiment of the selection systems provided herein, an inhibitor-resistant or altered enzyme of a pyrimidine biosynthesis pathway is used as a selectable marker. The enzyme may be a rate-limiting enzyme of de novo pyrimidine nucleotide biosynthesis. For example, an inhibitor-resistant or altered dihydroorotate dehydrogenase (DHODH) enzyme (particularly a mammalian, such as rodent and human, DHODH), which serves as a selectable marker, is used to confer on cells in which it is expressed resistance to an inhibitor of DHODH (i.e., a selection agent). If the selectable marker is an altered DHODH, the alteration in the DHODH is such that the enzyme is no longer effectively inhibited by the selection agent.

Inhibitor-resistant DHODH enzymes are DHODH enzymes that are resistant to one or more conditions or agents that are effective inhibitors of other DHODH enzymes and thus include altered DHODH enzymes. Altered DHODH enzymes include DHODH enzymes having alterations relative to the amino acid sequences set forth in SEQ. ID. NOS. 20, 22 and 16 that impart resistance on the enzyme to a DHODH inhibitor. Such altered enzymes include those containing substantially the same amino acid sequence as set forth in SEQ. ID. NOS. 26 and 28. Exemplary nucleic acid encoding such sequences includes the sequence set forth in SEQ. ID. NOS. 25 and 27, which are sequences encoding mutant A. nidulans DHODH enzymes resistant to CCFQ. Other altered DHODH enzymes include enzymes containing substantially the same amino acid sequence as set forth in SEQ. ID. NO. 20 or SEQ. ID. NO. 22 except that the amino acid at position 134 or 105, respectively, is other than valine, e.g., glutamic acid or conservative amino acid substitutions of glutamic acid. Exemplary nucleic acid encoding such sequences includes the sequence set forth in SEQ. ID. NOS. 19 (nucleotides 1-1188) and 21, except that the triplet of nucleotides 400-402 of SEQ. ID. NO. 19 and the triplet of nucleotides 313-315 of SEQ. ID. NO. 21 encodes an amino acid other than valine, e.g., glutamic acid or a conservative amino acid substitution of glutamic acid.

Inhibitor-resistant enzymes may be altered human DHODH enzymes containing substantially the same amino acid sequence as set forth in SEQ. ID. NO. 20 or SEQ. ID. NO. 22, except that amino acid 56 or 27, respectively, is other than histidine, e.g., alanine or a conservative substitution thereof. Exemplary nucleic acid encoding such sequences includes the sequence set forth in SEQ. ID. NOS. 19 (nucleotides 1-1188) and 21, except that the triplet of nucleotides 166-168 of SEQ. ID. NO. 19 and the triplet of nucleotides 79-81 of SEQ. ID. NO. 21 encodes an amino acid other than histidine, e.g., alanine or a conservative amino acid substitution of alanine. Included among such enzymes is one containing substantially the same amino acid sequence as set forth in SEQ. ID. NO. 24. Exemplary nucleic acid encoding this sequence includes the sequence of nucleotides 4-1101 set forth in SEQ. ID. NO. 23.

Inhibitor-resistant enzymes (e.g., inhibitor-resistant IMPDH and DHODH) are insensitive to or have reduced sensitivity to one or more inhibitors of the enzymes. Some inhibitor-resistant or altered enzymes may be less sensitive to inhibitor than inhibitor-sensitive or wild-type enzymes by a factor of about 1.5-fold to 2500 (or more)-fold or about 3-fold to 2500-fold or about 3-fold to 500-fold or about 5-fold to 200-fold. Sensitivity can be expressed, for example, as the IC₅₀ value (i.e., the inhibitor concentration that effects half-maximal inhibition, which will be greater for less sensitive enzymes).

The catalytic activity of altered enzymes which may be used as selectable markers is substantially similar to or at least not significantly reduced relative to the catalytic activity of wild-type enzymes. Some inhibitor-resistant or altered enzymes may have a Kᵢ for inhibitor that is in the range of about 1.5-fold to about 2500 (or more)-fold or about 3-fold to 2500-fold or about 3-fold to 500-fold greater than the Kᵢ; of the inhibitor-sensitive or wild-type enzyme for the inhibitor. Some altered enzymes may have a Kₘ for substrate and/or cofactor that is at least similar to that of the wild-type enzyme or within a range of about 1.5-fold to about 5-fold or about 1.5-fold to about 3-fold of the inhibitor-sensitive or wild-type enzyme. Some inhibitor-resistant or altered enzymes may have a k_{cat} value that is at least similar to that of the inhibitor-sensitive or wild-type enzyme or within a range of 0.1- to 0.9-fold of that of the inhibitor-sensitive or wild-type enzyme.

Inhibitor-resistant enzymes useful as selectable markers in particular selection systems described herein provide in cells in which they are contained increased resistance to inhibition of cell proliferation and/or reduction of cell viability by an inhibitor of the enzyme relative to the resistance of cells (in particular, at least substantially identically cells or cells of similar type) that do not contain the inhibitor-resistant enzyme (e.g., cells that contain an inhibitor-sensitive enzyme but no inhibitor-resistant enzyme). Thus, a higher concentration of inhibitor is required to achieve the same level of reduction in cell proliferation and/or viability in cells expressing an inhibitor-resistant enzyme, e.g., an inhibitor-resistant or altered IMPDH or DHODH, as compared to cells expressing a sensitive enzyme. Some inhibitor-resistant enzymes for use as selectable markers may provide in cells in which they are expressed about 2- to 10,000-fold or greater (typically about 5- to 2500-fold or 10- to 500-fold) increased resistance to inhibition of cell proliferation and/or reduction in cell viability by an inhibitor of the enzyme. This can be expressed, for example, as a 2- to 10,000-fold or greater (typically about 5- to 2500-fold or 10- to 500-fold) increase in the IC₅₀ value for growth inhibition and/or viability reduction for the inhibitor of the enzyme.

Increased resistance to inhibition of cellular proliferation and/or reduction of cell viability can be the result of increased Kᵢ of an inhibitor-resistant enzyme (as compared to a inhibitor-sensitive or unaltered or wild-type enzyme) or the combination of increased Kᵢ and increased expression (i.e., resulting in increased protein levels) of an inhibitor-resistant enzyme in resistant cells relative to the amount of expression of an inhibitor-sensitive enzyme in non-resistant cells. It is possible for increased resistance to inhibition of cell proliferation and/or reduction of cell viability to be achieved by virtue of the reduced sensitivity of the inhibitor-resistant enzyme for an inhibitor such that substantially increased expression is not required to effect selection of cells containing the inhibitor-resistant enzyme. Selection of genetically modified cells may be achieved in such selection systems through introduction and expression of as few as about 1 to about 10, or about 2 to about 10 or about 1 to about 5 or about 2 to about 5 or about 2 copies or 1 copy of a nucleic acid encoding an inhibitor-resistant enzyme into a cell. Thus, it is possible for increased resistance to inhibition of cell proliferation and/or reduction of cell viability to be achieved by virtue of the reduced sensitivity of the inhibitor-resistant enzyme to an inhibitor without depending on increased levels of expression of the inhibitor-resistant enzyme.

Enzyme inhibitors that may be used as selection agents in systems employing an inhibitor-resistant or altered enzyme as a selectable marker typically provide for a level of inhibition of sensitive or unaltered enzyme in cells such that at least certain cells exposed to the inhibitor under certain conditions exhibit reduced proliferation and/or viability relative to the amount of proliferation and/or viability of the cells in the absence of the inhibitor. At minimum, cells that may exhibit reduced proliferation and/or viability when exposed to the enzyme inhibitor are those that do not contain an inhibitor-resistant or altered enzyme that confers resistance to conditions that include the presence of the inhibitor, but that are otherwise substantially identical or similar to cells that contain the inhibitor-resistant or altered enzyme. Enzyme inhibitors that may be used as selection agents in selection systems provided herein generally are ineffective at substantially inhibiting an inhibitor-resistant or altered enzyme that may be used as a selectable marker.

A selectable marker provided herein may confer one or more properties on a cell containing or expressing the marker which may be a basis for distinguishing the cells and/or for differentially affecting cells containing and cells lacking the marker. Selectable markers thus can, for example, provide a basis for identifying, isolating, differentially affecting and/or providing a selective advantage for proliferation and/or viability of certain cells.

Methods are described herein for providing a cell with a selective advantage for proliferation and/or viability. In these methods, cells are provided with a selectable marker that gives the cells an advantageous position for cell survival and/or proliferation under conditions that inhibit and/or fail to provide for increases in the proliferation of and/or reduce the viability of cells that do not contain the selectable marker. The selective advantage for proliferation and/or viability of cells containing the selectable marker may be exploited in a variety of ways. In one method as provided herein, it may be used as a basis for identifying and/or isolating the marker-containing cells in or from a population of cells. The population of cells may be, for example, substantially identical cells except that some cells contain and some lack the selectable marker. For example, under the selection conditions, the marker-containing cells may exhibit the greatest viability (or be the only surviving cells), exhibit the most rapid proliferation (or be the only proliferating cells) and/or make up the largest segment of the cell population.

Provided herein are methods of providing a selective advantage for proliferation of a first cell relative to a second cell, by introducing a nucleic acid molecule encoding an altered mammalian inosine monophosphate dehydrogenase (IMPDH) and/or an altered mammalian dihydroorotate dehydrogenase (DHODH) into the first cell, wherein the altered IMPDH or DHODH is resistant to an inhibitor of purine biosynthesis and the first cell is a mammalian cell. For example, the nucleic acid molecule may encode the sequence of amino acids set forth in SEQ. ID. NO. 4 (except that amino acids 190 and 191 are alanine and glycine, respectively) and may include the sequence of nucleotides 89 to 1589 in SEQ. ID. NO. 3 (or may include nucleotides 48 to 1589 of SEQ. ID. NO. 3 except that the sequence of nucleotides 614-619 is TGCAGG instead of CCGCAG). Altered IMPDH enzymes include altered IMPDH type I and type II enzymes having alterations relative to the amino acid sequences set forth in SEQ. ID. NOS. 2, 18 and 30 that impart resistance on the enzyme to an IMPDH inhibitor. Included among such altered IMPDH enzymes are those that contain one or more alterations in the sequence of amino acids 330-451, 330-441, 330-400, 330-355 or 333-351 of SEQ. ID. NOS. 2, 18 and 30. Particular alterations of the amino acid sequence set forth in SEQ. ID. NO. 2 include one or more of the following: sequences in which amino acid 333 is other than threonine, amino acid 351 is other than serine, amino acid 277 is other than glutamine, amino acid 462 is other than alanine, amino acid 456 is other than phenylalanine and amino acid 470 is other than aspartic acid. Particular alterations of the amino acid sequence set forth in SEQ. ID. NO. 30 include sequences in which amino acid 333 is other than threonine and/or amino acid 351 is other than serine. One such altered IMPDH is a mutant mouse IMPDH type II (see SEQ ID NO. 32). A nucleotide sequence encoding this altered mouse type II IMPDH is provided in SEQ ID NO. 31.

Altered human IMPDH enzymes may also contain one or more alterations in:

(a) the sequence of amino acids from a position corresponding to amino acid 330 to a position corresponding to amino acid 451 of SEQ. ID. NO. 2;

(b) the sequence of amino acids from a position corresponding to amino acid 330 to a position corresponding to amino acid 441 of SEQ. ID. NO. 2;

(c) the sequence of amino acids from a position corresponding to amino acid 330 to a position corresponding to amino acid 400 of SEQ. ID. NO. 2;

(d) the sequence of amino acids from a position corresponding to amino acid 330 to a position corresponding to amino acid 355 of SEQ. ID. NO. 2; or

(e) the sequence of amino acids from a position corresponding to amino acid 333 to a position corresponding to amino acid 351 of SEQ. ID. NO. 2; whereby the altered IMPDH is resistant to a purine biosynthesis inhibitor. In a particular embodiment, the altered human IMPDH enzyme is resistant to mycophenolic acid or a derivative, analog or metabolite thereof. For example, an altered human IMPDH enzyme may contain one or more alterations in the sequence of amino acids from a position corresponding to amino acid 333 to a position corresponding to amino acid 351 of SEQ. ID. NO. 2. Examples of such enzymes are those containing an amino acid other than threonine at a position corresponding to amino acid 333 of SEQ. ID. NO. 2 or an amino acid other than serine at a position corresponding to amino acid 351 of SEQ. ID. NO. 2 or an amino acid other than threonine at a position corresponding to amino acid 333 of SEQ. ID. NO. 2 and an amino acid other than serine at a position corresponding to amino acid 351 of SEQ. ID. NO. 2. Particular enzymes are those wherein the amino acid other than threonine at a position corresponding to amino acid 333 is isoleucine or a conservative substitution thereof and the amino acid other than serine at a position corresponding to amino acid 351 is tyrosine or a conservative substitution thereof. One such enzyme provided herein has substantially the same sequence of amino acids as set forth in SEQ ID NO. 4 (or as set forth in SEQ. ID. NO. 4 except that amino acids 190 and 191 are alanine and glycine, respectively). A nucleotide sequence encoding this altered human IMPDH includes nucleotides 48 to 1589 of SEQ. ID. NO. 3 (or may include nucleotides 48 to 1589 of SEQ. ID. NO. 3 except that the sequence of nucleotides 614-619 is TGCAGG instead of CCGCAG).

Described herein are methods of providing for selective proliferation, viability or proliferation and viability of a first cell relative to a second cell by introducing into the first cell a nucleic acid encoding an altered mammalian enzyme and exposing the first and second cells to conditions that inhibit the unaltered mammalian enzyme but to which the altered mammalian enzyme is resistant; whereby the first cell exhibits greater proliferation, viability or proliferation and viability relative to the second cell. The mammalian enzyme may be from any of a variety of species, including but not limited to rodents, e.g., mouse or rat, and humans. Any cells may be used in the methods, including, but not limited to, eukaryotic cells and, particularly, mammalian and human cells.

Also provided herein are methods of providing for selective proliferation, viability or proliferation and viability of a first cell relative to a second cell, by introducing a nucleic acid encoding an altered mammalian enzyme of a nucleotide biosynthesis pathway into the first cell and exposing the first and second cells to conditions that inhibit unaltered mammalian enzyme but to which the altered mammalian enzyme is resistant; whereby the first cell exhibits greater proliferation, viability or proliferation and viability relative to the second cell. The mammalian enzyme may be, for example, an enzyme of a purine nucleotide biosynthesis pathway, such as a mammalian, e.g., rodent or human, inosine monophosphate dehydrogenase (IMPDH), or an enzyme of a pyrimidine nucleotide biosynthesis pathway, such as a mammalian, e.g., rodent or human, dihydroorotate dehydrogenase (DHODH). One or more such enzymes may be used in the methods, and in any combination.

In particular embodiments of these methods of providing for selective proliferation, viability or proliferation and viability, the mammalian enzyme may be, for example, IMPDH, ribose phosphate pyrophosphokinase, amidophosphoribosyltransferase, glycinamide ribonucleotide (GAR) synthetase, GAR transformylase, formylglycinamidine ribonucleotide (FGAM) synthetase, aminoimidazole ribonucleotide (AIR) synthetase, AIR carboxylase, aminoimidazolesuccinocarboxamide ribonucleotide (SAICAR) synthetase, adenylosuccinate synthase, adenylosuccinate lyase, aminoimidazolecarboxamide ribonucleotide (AICAR) transformylase, inosine monophosphate (IMP) cyclohydrolase and GMP synthase. Any of these mammalian enzymes may be from, for example, a rodent or a human.

In a further embodiment of these methods of providing for selective proliferation, viability or proliferation and viability, the mammalian enzyme is an IMPDH, for example, an IMPDH type I or type II. Because different cells, as well as the same cells under differing conditions, differentially utilize the two IMPDH isoforms and the de novo and salvage pathways for purine biosynthesis, selection systems using an inhibitor-resistant or altered IMPDH as a selectable marker may be designed in a variety of formats. For example, the IMPDH may be an IMPDH type II and the conditions to which the first and second cells are exposed may include exposure to an inhibitor of IMPDH type II (such as, for example, one or more of mycophenolic acid, mycophenolate mofetil, ribavirin, tiazofurin, 5-ethynyl-1-,β-D-ribofuranosylimidazole-4-carboxamide, mizoribine, selanazole-4-carboxamide adenine dinucleotide, pyridazines and VX-497 or derivatives, analogs or metabolites of any of the foregoing). This format is particularly useful when the predominant IMPDH isoform expressed in the cells is IMPDH type II, such as in lymphocytes, cancer cells, tumor cells, leukemic cells, proliferating cells, and mesangial cells. When the cells are T-lymphocytes or B-lymphocytes, they may be activated prior to exposure to an inhibitor of IMPDH type II. The cells may be exposed to one or more compositions selected from the group consisting of antigens, antibodies, cytokines, growth factors and mitogens. Alternatively, the conditions may include exposure to an inhibitor of type I IMPDH, which may be of particular use when the predominant isoform expressed by the cells is an IMPDH type I. The conditions may include exposure to an inhibitor of IMPDH type I and type II or to an inhibitor of IMPDH type I and an inhibitor of IMPDH type II, such as mycophenolic acid or a derivative, analog or metabolite thereof.

In another embodiment of these methods of providing for selective proliferation, viability or proliferation and viability, the IMPDH may be an IMPDH type I. The conditions may include exposure to an inhibitor of IMPDH type I and/or an inhibitor of IMPDH type II. Exposure to an inhibitor of IMPDH type II may be of particular use when the predominant IMPDH isoform expressed in the cells is IMPDH type II. Exposure to an inhibitor of IMPDH type I may be of particular use when the predominant isoform expressed in the cells is IMPDH type I. The conditions may also include exposure to an inhibitor of IMPDH type I and type II.

In another embodiment of these methods of providing for selective proliferation, viability or proliferation and viability, the nucleic acid introduced into the first cell includes nucleic acid encoding an altered IMPDH type I and nucleic acid encoding an altered IMPDH type II and the conditions include exposure to an inhibitor of IMPDH type I and type II or to an inhibitor of IMPDH type I and an inhibitor of IMPDH type II. In any of these methods of providing for selective proliferation, viability or proliferation and viability, which include introduction of an altered mammalian IMPDH into cells, the conditions may include exposure to one or more inhibitors of IMPDH and one or more inhibitors of a purine salvage pathway enzyme, for example, allopurinol. This format is particularly useful when the cells have sufficient purine synthesis via the salvage pathway and, thus, may not be sufficiently inhibited in their proliferation and/or viability by exposure to IMPDH inhibitors.

In particular embodiments of these methods of providing for selective proliferation, viability or proliferation and viability, the mammalian enzyme is an enzyme of a pyrimidine nucleotide biosynthesis pathway, for example, DHODH. Because different cells, as well as the same cells under differing conditions, differentially utilize the de novo and salvage pathways for pyrimidine biosynthesis, selection systems using an inhibitor-resistant or altered enzyme of a pyrimidine nucleotide biosynthesis pathway as a selectable marker may be designed in a variety of formats. For example, the mammalian enzyme may be from a variety of mammalian species, including, but not limited to rodents, e.g., mouse and rat, and humans. Any cells may be used in the methods, including, but not limited to, eukaryotic cells and, particularly, mammalian and human cells.

Cells that may rely predominantly on the de novo pathway for pyrimidine (e.g., uridine) nucleotide synthesis include cells that can exhibit increased proliferation (e.g., lymphocytes, T- and B-lymphocytes, cancer cells, tumor cells, leukemic cells, and mesangial cells) and cells that exhibit known regenerative, absorptive or excretory activities (e.g., mucosal cells of the ileum, colon crypts in the gastrointestinal tract, cultured Ehrlich ascites tumor cells, and proximal tubule of the kidney cortex). Use of such cells in the methods provided herein may include an altered or inhibitor-resistant enzyme of the de novo pyrimidine biosynthesis pathway, e.g., an altered DHODH, as a selectable marker and an inhibitor of an enzyme of the de novo pathway [e.g., one or more inhibitors of DHODH, such as cinchoninic acid, brequinar (6-fluoro-2-(2′-fluoro-1,1′-biphenyl-4-yl)-3-methyl-4-quinoline carboxylic acid), naphthoquinone derivatives such as dichloroally lawsone, isoxazole derivatives such as leflunomide (N-(4-trifluoromethylphenyl)-5-methylisoxazol-4-carboxamide), quinolone carboxylic acids, naphthoquinones, isoxazoles, phenoxyquinolines, redoxal and derivatives, lawsone, lapachol, atovaquone and (8-chloro-4-(2-chloro-4-fluoro-phenoxy)quinoline) analogs or metabolites of any of the foregoing compositions] in the conditions to which cells are exposed. In these methods, the cells may be activated (e.g., by exposure to antigens, antibodies, cytokines, growth factors and mitogens) prior to exposure to an inhibitor.

Cells (such as, for example, cells of the periportal area of the liver, testis and spermatozoa, prostate and other glands, skeletal muscle, and some cancer cells) that are able to utilize the salvage pathway to synthesize pyrimidines, have lower DHODH activity, and/or that are not subject to substantial increased proliferation, may not be significantly affected by inhibitors of de novo pathway enzymes (e.g., DHODH inhibitors). Use of such cells in the methods provided herein may include an altered or inhibitor-resistant enzyme of the salvage pathway for pyrimidine biosynthesis, e.g., an altered thymidine kinase, deoxycytidine kinase, uridine kinase and/or cytidine kinase, as a selectable marker, and an inhibitor of such enzymes (e.g., AZT, cytosinearabinoside (Ara C), 2′2′-difluorodeoxycytidine (gemcitabine), camptothecin and analogs thereof, okadaic acid, organoselenium compounds and natriuretic factor and analogs thereof) in the conditions to which cells are exposed. Regardless of the type of cell used in any of these methods which utilize an altered mammalian enzyme of a pyrimidine biosynthesis pathway, the conditions may comprise exposure to one or more inhibitors of an enzyme of the de novo pathway (e.g., DHODH) and one or more inhibitors of a pyrimidine salvage pathway enzyme.

In another embodiment of the methods of providing for selective proliferation, viability or proliferation and viability, the nucleic acid introduced into the first cell includes nucleic acid encoding an inhibitor-resistant or altered enzyme of the purine salvage pathway, such as hypoxanthine guanine phosphoribosyltransferase (HGPRT) and may further include nucleic acid encoding an inhibitor resistant or altered enzyme of the de novo purine synthesis pathway, e.g., IMPDH. The conditions to which the first and second cells are exposed in such methods may include exposure to an inhibitor of one or more enzymes of the purine salvage pathway.

In another embodiment of the methods of providing for selective proliferation, viability or proliferation and viability, the nucleic acid introduced into the first cell includes nucleic acid encoding an inhibitor-resistant or altered enzyme of the pyrimidine salvage pathway, such as thymidine kinase, deoxycytidine kinase, uridine kinase and/or cytidine kinase and may further include nucleic acid encoding an inhibitor resistant or altered enzyme of the de novo pyrimidine synthesis pathway, e.g., DHODH. The conditions to which the first and second cells are exposed in such methods may include exposure to an inhibitor of one or more enzymes of the pyrimidine salvage pathway.

In any of these methods of providing for selective proliferation, viability or proliferation and viability, the nucleic acid encoding an altered mammalian enzyme may be introduced into the first cell in vitro or in vivo. When the first cell is in an organism, the altered enzyme, e.g., an altered IMPDH and/or DHODH, it may be advantageous for the altered enzyme to be minimally immunogenic in the organism. The nucleic acid encoding an altered mammalian enzyme may also be introduced into the first cell ex vivo and then transferred in the first cell and/or its progeny into an organism. In this method, it also may be advantageous for the altered enzyme, e.g., an altered IMPDH and/or DHODH, to be minimally immunogenic in the organism. In either of these methods in which a cell containing an altered enzyme-encoding nucleic acid is present within an organism, a variety of organisms is contemplated, including, but not limited to mammals, such as rodents and humans.

Any of these methods of providing for selective proliferation, viability or proliferation and viability may include introducing heterologous nucleic acid into the first cell in addition to nucleic acid encoding an altered mammalian enzyme e.g., IMPDH and/or DHODH. In such methods, the selective proliferation and/or viability of cells that have taken up nucleic acid encoding an altered mammalian enzyme facilitates identifying, isolating and differentially affecting cells that have taken up the additional heterologous nucleic acid and that may stably express the heterologous nucleic acid. Nucleic acid encoding an altered mammalian enzyme (e.g., IMPDH and/or DHODH) and a heterologous nucleic acid of interest are co-transfected into the cells. The co-transfection may be simultaneous (i.e., cells are exposed to nucleic acid encoding an altered mammalian enzyme and the nucleic acid of interest in the same transfection procedure) or sequential in which two or more transfection procedures are conducted for separate transfer of the nucleic acid encoding the altered mammalian enzyme and the heterologous nucleic acids of interest into the host cells.

In any of these methods of providing for selective proliferation, viability or proliferation and viability, the conditions to which the cells are exposed may be such that they do not substantially affect cells that are not similar or identical to the first cell into which the nucleic acid encoding an altered mammalian enzyme is introduced. Such methods are particularly advantageous when the first cell is exposed to the conditions in vivo in an organism where any substantial reduction in the proliferation and/or viability of a wide variety of cells could be detrimental or lethal to the organism. For example, if the first cell is a proliferating lymphocyte, e.g., a T-lymphocyte, and is exposed within an organism to one or more inhibitors of a de novo nucleotide biosynthesis pathway enzyme, e.g., IMPDH and/or DHODH, to which it is resistant due to the presence of one or more altered enzymes, only a limited number of cells (e.g., only highly proliferating cells within the organism that do not utilize salvage pathways to maintain the highly proliferative state, such as proliferating T-lymphocytes that do not contain the altered enzyme) may exhibit a reduction in proliferation and/or viability. The majority of the organism's cells will not be substantially affected by the inhibitor.

In any of these methods of providing for selective proliferation, viability or proliferation and viability, wherein the altered mammalian enzyme is an enzyme of a nucleotide biosynthesis pathway, purine and pyrimidine biosynthesis pathway enzymes may be used independently or in combination. Thus, for example, inhibitor-resistant enzymes of the purine and pyrimidine biosynthesis pathways may be used together as selectable markers in systems which include the corresponding inhibitors of purine and pyrimidine biosynthesis pathway enzymes in the conditions to which cells are exposed (i.e, as selection agents). One advantage of such systems is the ability to conduct multiple selections for transfer of multiple heterologous nucleic acids into host cells. Inhibitor-resistant enzymes of the de novo purine and pyrimidine biosynthesis pathways also may be used as selectable markers in various combinations with de novo pathway inhibitors and purine and pyrimidine salvage pathway inhibitors as selection agents.

In methods as provided herein, the selective advantage for proliferation and/or viability may be used as a basis for differentially affecting the marker-containing cells. The ability to differentially affect marker-containing cells finds particular use in transfer of cells into immunosuppressed hosts, for example, in the adoptive transfer of T cells in conjunction with allogeneic bone marrow transplantation and treatment of immune disorders, such as treatments that might include transplantation of allogeneic stem cells containing a required functional gene. In such methods, cells transferred into a host may be provided with a selectable marker, e.g., nucleic acid encoding an inhibitor-resistant IMPDH or DHODH, which confers resistance to an immunosuppressant, e.g., an inhibitor of IMPDH (such as mycophenolic acid) or DHODH, administered to the host. The marker-containing cells will not be substantially affected by the immunosuppressant, whereas proliferation and/or viability of the host's own immune cells, e.g., T cells, will be reduced. Thus, the transferred cells will exhibit greater proliferation and/or viability relative to the host's immune cells.

Provided herein are methods of providing for selective proliferation, viability or proliferation and viability of a first cell relative to a substantially identical or similar type second cell in vivo by introducing a nucleic acid molecule into the first cell and exposing the first and second cells in vivo to conditions that inhibit the proliferation and/or reduce the viability of the substantially identical or similar type second cell, whereby the first cell exhibits greater proliferation, viability or proliferation and viability relative to the second cell. In these methods, the nucleic acid molecule confers resistance to the conditions on the first cell, and the conditions primarily directly affect only cells similar to the first cell that do not contain the nucleic acid.

In these methods of providing for selective proliferation, viability or proliferation and viability in vivo, the nucleic acid molecule that confers resistance to the conditions on the first cell may encode, for example, one or more resistant or altered enzymes of nucleotide biosynthesis pathways. Examples of such enzymes include resistant or altered enzymes of a purine nucleotide biosynthesis pathway, such as inosine monophosphate dehydrogenase (IMPDH) and others, including others described herein, or of a pyrimidine nucleotide biosynthesis pathway, such dihydroorotate dehydrogenase (DHODH) and others, including others described herein, or of salvage pathway, e.g., hypoxanthine guanine phosphoribosyltransferase (HGPRT), thymidine kinase, deoxycytidine kinase, uridine kinase and/or cytidine kinase. One or more such enzymes may be used in the methods, and in any combination. Cells that may be used in these methods include, but are not limited to mammalian cells such as rodent and human cells and in particular lymphocytes, such as T- and B-lymphocytes.

Conditions to which the cells are exposed in vivo which primarily directly affect only cells similar to the first cell that do not contain the nucleic acid include exposure to one or more inhibitors of enzymes of nucleotide biosynthesis pathways. For example, such inhibitors include the following and derivatives, analogs and metabolites thereof: mycophenolic acid, mycophenolate mofetil, ribavirin, tiazofurin, 5-ethynyl-1-β-D-ribofuranosylimidazole-4-carboxamide, mizoribine, selanazole-4-carboxamide adenine dinucleotide, pyridazines, VX-497, cinchoninic acid, brequinar (6-fluoro-2-(2′-fluoro-1,1′-biphenyl-4-yl)-3-methyl-4-quinoline carboxylic acid), naphthoquinone derivatives such as dichloroally lawsone, isoxazole derivatives such as leflunomide (N-(4-trifluoromethylphenyl)-5-methylisoxazol-4-carboxamide), quinolone carboxylic acids, naphthoquinones, isoxazoles, phenoxyquinolines, redoxal and derivatives, lawsone, lapachol, atovaquone and (8-chloro-4-(2-chloro-4-fluoro-phenoxy)quinoline).

In these methods of providing for selective proliferation, viability or proliferation and viability in vivo, it is particularly advantageous for the nucleic acid that confers resistance to the conditions to encode a protein that is minimally immunogenic in vivo in an organism.

In these methods of providing for selective proliferation, viability or proliferation and viability in vivo, the nucleic acid molecule that confers resistance to the conditions may be introduced into the first cell in vivo in an organism, or may be introduced into the first cell ex vivo and further include transfer of the first cell containing the nucleic acid and/or its progeny into an organism.

In particular embodiments of these methods of providing for selective proliferation and/or viability in vivo, the conditions include administering to the organism an immunosuppressive agent. The immunosuppressive agent may include an inhibitor of one or more enzymes of one or more nucleotide biosynthetic pathways.

The methods may be used in connection with selective proliferation of numerous cell types, including, but not limited to lymphocytes, such as T- and B-lymphocytes, and stem cells.

Any of the methods of providing selective proliferation and/or viability in vivo find particular use in instances in which the organism has undergone a bone marrow or solid organ transplantation.

Any of these methods of providing for selective proliferation and/or viability in vivo may include introducing heterologous nucleic acid into the first cell in addition to nucleic acid that confers resistance to the conditions to which the cells are exposed.

Provided herein are methods for transferring a heterologous nucleic acid molecule into an organism by introducing nucleic acids containing the heterologous nucleic acid molecule and a marker nucleic acid molecule into a first cell in the organism and exposing the organism to conditions that inhibit the proliferation and/or reduce viability of a substantially identical or similar type cell that does not contain the marker nucleic acid. In these particular methods, the marker nucleic acid molecule confers resistance to the conditions on the first cell and the conditions primarily directly affect only cells similar to the first cell that do not contain the marker nucleic acid.

Further provided are methods for transferring a heterologous nucleic acid molecule into an organism by introducing nucleic acids containing the heterologous nucleic acid molecule and a marker nucleic acid molecule into a first cell, introducing the first cell into the organism, and exposing the organism to conditions that inhibit the proliferation and/or reduce viability of a substantially identical or similar type cell that does not contain the marker nucleic acid. In these particular methods, the marker nucleic acid molecule confers resistance to the conditions on the first cell and the conditions primarily directly affect only cells similar to the first cell that do not contain the marker nucleic acid.

In any of these methods for transferring a heterologous nucleic acid molecule into an organism, the heterologous nucleic acid molecule may encode a therapeutic product or in some way be involved in providing a therapeutic benefit within the organism. For example, the heterologous nucleic acid molecule may encode a product that alters the organism's immune responses (e.g., an immunomodulatory, anti-inflammatory or protective protein) and the conditions may include administering to the organism an immunosuppressive agent (e.g., an inhibitor of one or more enzymes of one or more nucleotide biosynthetic pathways). Such methods may be useful in connection with treating an immune disorder of the organism. In particular embodiments, the first cell is a lymphocyte, such as a T- or B-lymphocyte.

Resistant or altered enzymes, or nucleic acids encoding resistant or altered enzymes, as referred to throughout all of the many methods provided herein, may include any one or more of the above-referenced enzymes and nucleic acids as well as any of the following:

nucleic acid encoding the amino acid sequence set forth in SEQ. ID. NO. 32;

nucleic acid containing the sequence of nucleotides 67 to 1611 in SEQ. ID. NO. 31;

nucleic acid encoding the amino acid sequence set forth in SEQ. ID. NO. 30 except that the codon for amino acid 333 encodes an animo acid other than threonine;

nucleic acid encoding the amino acid sequence set forth in SEQ. ID. NO. 30 except that the codon for amino acid 351 encodes an animo acid other than serine;

nucleic acid encoding the amino acid sequence set forth in SEQ. ID. NO. 30 except that the codon for amino acid 333 encodes an amino acid other than threonine and the codon for amino acid 351 encodes an animo acid other than serine;

nucleic acid encoding the amino acid sequence set forth in SEQ. ID. NO. 2 except that the codon for amino acid 333 encodes an animo acid other than threonine;

nucleic acid encoding the amino acid sequence set forth in SEQ. ID. NO. 2 except that the codon for amino acid 351 encodes an animo acid other than serine;

nucleic acid encoding the amino acid sequence set forth in SEQ. ID. NO. 2 except that the codon for amino acid 333 encodes an amino acid other than threonine and the codon for amino acid 351 encodes an animo acid other than serine;

nucleic acid encoding the amino acid sequence set forth in SEQ. ID. NO. 4 (or as set forth in SEQ. ID. NO. 4 except that amino acids 190 and 191 are alanine and glycine, respectively);

nucleic acid containing the sequence of nucleotides 48 to 1589 in SEQ. ID. NO. 3 (or the sequence of nucleotides 48 to 1589 of SEQ. ID. NO. 3 except that the sequence of nucleotides 614-619 is TGCAGG instead of CCGCAG);

nucleic acid encoding the amino acid sequence set forth in SEQ. ID. NO. 2, except that the codon for amino acid 277 encodes an amino acid other than glutamine;

nucleic acid encoding the amino acid sequence set forth in SEQ. ID. NO. 2, except that the codon for amino acid 462 encodes an amino acid other than alanine;

nucleic acid encoding the amino acid sequence set forth in SEQ. ID. NO. 2, except that the codon for amino acid 277 encodes an amino acid other than glutamine and the codon for amino acid 462 encodes an amino acid other than alanine;

nucleic acid encoding the amino acid sequence set forth in SEQ. ID. NO. 2, except that the codon for amino acid 456 encodes an amino acid other than phenylalanine and the codon for amino acid 470 encodes an amino acid other than aspartic acid;

nucleic acid encoding the amino acid sequence set forth in any of SEQ. ID. NO. 6, SEQ. ID. NO. 8, SEQ. ID. NO. 10 and SEQ. ID. NO. 12;

nucleic acid containing the sequence of nucleotides 48 to 189 in any of SEQ. ID. NO. 5, SEQ. ID. NO. 7, SEQ. ID. NO. 9 and SEQ. ID. NO. 11;

nucleic acid encoding the amino acid sequence set forth in SEQ. ID. NO. 24;

nucleic acid containing the sequence of nucleotides 4 to 1101 in SEQ. ID. NO. 23;

nucleic acid encoding the amino acid sequence set forth in SEQ. ID. NO. 22 except that the codon for amino acid 26 encodes an animo acid other than histidine;

nucleic acid encoding the amino acid sequence set forth in SEQ. ID. NO. 20 except that the codon for amino acid 56 encodes an animo acid other than histidine;

nucleic acid encoding the amino acid sequence set forth in SEQ. ID. NO. 22 except that the codon for amino acid 105 encodes an amino acid other than valine;

nucleic acid encoding the amino acid sequence set forth in SEQ. ID. NO. 22 except that the codon for amino acid 105 encodes a glutamic acid residue;

nucleic acid encoding the amino acid sequence set forth in SEQ. ID. NO. 20 except that the codon for amino acid 134 encodes an amino acid other than valine;

nucleic acid encoding the amino acid sequence set forth in SEQ. ID. NO. 20 except that the codon for amino acid 134 encodes a glutamic acid residue.

### DESCRIPTION OF THE DRAWINGS

FIG. 1 depicts the nucleotide sequence of plasmid IMPDH(1)Hytk/pMG\hat{ }Pac containing DNA encoding an altered human IMPDH (T333I/S351Y) within plasmid pMG.

### DETAILED DESCRIPTION OF PREFERRED EMBODIMENTS

Definitions

Unless defined otherwise, all technical and scientific terms used herein have the same meaning as is commonly understood by one of skill in the art to which this invention belongs. All patents, patent applications and publications referred to herein are incorporated by reference.

As used herein, “selection” refers to a process of subjecting cells to conditions which differentially affect and/or distinguish cells containing a selectable marker and cells that do not contain a selectable marker. Selection can be used, for example, in isolating, identifying and/or selectively affecting cells. In a particular selection process, cells are subjected to conditions under which cells containing a selectable marker exhibit greater proliferation and/or viability relative to the proliferation and/or viability of cells that do not contain the selectable marker.

As used herein, a “selectable marker” is a composition that confers one or more properties on a cell containing or expressing the marker which may be a basis for distinguishing the cells and/or for differentially affecting cells containing and cells lacking the marker. Selectable markers thus can, for example, provide a basis for identifying, isolating, differentially affecting and/or providing a selective advantage for proliferation and/or viability of certain cells. A selectable marker can be a composition, such as, for example, a protein, chemical compound, carbohydrate or lipid, that directly provides for identification, isolation and/or a selective advantage for proliferation, or can be a precursor to or component involved in the generation of a composition that directly provides for identification, isolation and/or a selective advantage for proliferation.

As used herein, a “selection agent” is a composition and/or condition to which cells may be exposed that differentially affects different cells and/or distinguishes certain cells. Thus, for example, upon exposure of cells to a selection agent, some of the cells may exhibit greater proliferation and/or viability than other cells.

As used herein, “conditions” to which cells may be exposed or subjected include, but are not limited to, environmental factors, such as, for example, temperature, light, pH, radiation and pressure, and chemical compositions or agents, such as, for example, drugs, enzyme inhibitors, chemotherapeutic agents and antibiotics, and combinations thereof.

As used herein, “proliferation” with respect to cells refers to cell growth via cell division or replication. Increases or decreases in cell proliferation can refer to processes in which the replication rate and/or absolute cell number is increased or decreased. Cell proliferation, which is distinct from viability, can be determined using methods known to those of skill in the art, including, for example, by measuring [³H]thymidine incorporation into cells as an assessment of DNA synthesis and an indicator of cell replication and by cell counting using a hemacytometer.

As used herein, “viability” with respect to cells refers to the ability of a cell to maintain its existence. Methods of assessing cell viability are known to those of skill in the art and include, but are not limited to, methods using indicators of cell function. For example, the ability of cells to exclude certain dyes, such as trypan blue, which accumulate in dead cells may be used to assess cell viability. Cell viability may be assessed by using fluorogenic reagents that distinguish live and dead cells (see, e.g., U.S. Pat. No. 5,314,805). Evidence of cell viability may also be based on assessments of basal metabolism or cell proliferation. The demonstration of synthesis of cell products (e.g., uptake of labeled amino acids into newly synthesized protein) is also indicative of cell viability.

Cell membrane integrity is commonly used to indicate cell viability. Loss of the cell membrane results in loss of cell structure, critical cell contents, essential ionic gradients and electrical potential. Major losses of membrane integrity are therefore associated with cell death. While there is not an exact equivalence between an intact cell membrane and cell viability, it is common to refer to cells that have intact membranes as viable (living) cells and cells where the membrane has been irreversibly disrupted, for example by a cytotoxic reagent, as dead (nonviable) cells.

As used herein, the phrase “selective advantage for the proliferation of cells” refers to a more favorable position of certain cells for proliferation relative to other cells. A selective advantage for proliferation of certain cells may be provided by the introduction of a selectable marker into the cells. The selective advantageous proliferation of cells containing the selectable marker relative to cells that do not contain the marker may be the result of a number factors including, but not limited to, inhibition of the proliferation of cells that do not have a selective advantage and/or stimulation of the proliferation of cells that do have a selective advantage. The selective advantage for proliferation of cells may be relative to the proliferation of some, but not a wide variety of, other cells that do not have a selective advantage.

Cells that have a selective advantage for proliferation may exhibit greater proliferation relative to other cells that do not have a selective advantage. Greater proliferation may be, for example, a greater rate of replication and/or a greater number of replicated cells. Thus, cells that have a selective advantage for proliferation may be identified or isolated from cells that do not have a selective advantage for proliferation by virtue of the selective, greater proliferation of the cells having a selective advantage.

As used herein, “inhibition of proliferation of cells” or “inhibition of cell proliferation” refers to a reduction in the proliferation of cells, as, for example, by decreasing the rate of replication and/or the number of replicated cells. Inhibition may be essentially complete, whereby few to no cells would remain, or partial, wherein the number of cells may be reduced but the cells are not eliminated in total.

As used herein, “activity” with reference to an enzyme refers to the involvement of enzymes in chemical reactions. Enzyme activity can be catalytic activity in which an enzyme increases reaction rates by reducing the activation energy of a particular reaction. Enzyme activity can include binding of enzyme to other molecules, for example, substrate and cofactor molecules. In living cells, enzymes can make reactions take place at a rate that is conducive to sustaining cell viability and promoting cell growth. Enzymes can be part of a series of reactions, e.g., a reaction pathway, that functions to yield a particular result, for example a product, such as a nucleotide, resulting from a biosynthetic pathway.

As used herein, “inhibition” of an enzyme refers to reduction in the activity of the enzyme.

As used herein, the phrase “substantially identical” with reference to a comparison of cells means that the compared cells are of the same type and have not been altered relative to one another in a manor that results in any significant differences between the compared cells, except as may be noted.

As used herein, the term “type” with reference to a cell means a category of cells that share one or more common features or characteristics. For example, cell types include highly proliferative cells, immune system cells, lymphocytes, T-lymphocytes, hematopoietic cells, bone marrow cells, cancer cells, malignant cells and others.

As used herein, the term “lymphocytes” refers to immune system cells derived from stem cells located within hematopoietic tissues. Included among lymphocytes are T-lymphocytes, or T cells, and B-lymphocytes, or B cells. T-lymphocytes develop from precursors in the thymus while B-lymphocytes develop in the bone marrow.

Lymphocytes recognize and bind to antigens which can induce rapid proliferation of the lymphocytes. B cells express surface receptors specific for a particular antigen and combat extracellular pathogens by releasing antibodies. T cells recognize antigens presented on cell (i.e., antigen-presenting cells or APC) surfaces by major histocompatibility complex (MHC) molecules. T cells use antigen-specific receptors (TCRs) to recognize antigenic peptides bound to MHC molecules. Categories of T cells include helper, cytotoxic and suppressor T cells.

Lymphocytes express a large number of different molecules on their surfaces which can be used to mark cell subsets. For example, lineage markers identify a specific lineage, such as CD3, found only on T cells. The CD4 antigen is expressed by helper T cells which perform helper or regulatory functions. CD8 antigen is expressed by cytotoxic and suppressor T cells.

As used herein, the term “cancer” or “cancerous” with respect to a cell refers to a cell that exhibits hallmarks of a cancer cell, including, but not limited to, loss of contact inhibition and immortality.

As used herein, the term “similar” with reference to a comparison of cells means that the compared cells are of the same or related types.

As used herein, the phrase “primarily directly affect only cells similar to . . . ” with reference to conditions means that although the conditions may have some limited and/or remote, indirect effects on multiple cell types, they principally have direct, substantial effects only on certain cells, e.g., cells similar to those into which a selectable marker is added. An example of a direct, substantial effect is a significant inhibition of proliferation and/or viability of cells as a result of exposure to the conditions. In contrast, a limited and/or remote, indirect effect is one which does not significantly, adversely impact on cells and/or organisms containing the cells.

Thus, for example, conditions used in vivo in an organism which primarily directly affect only a specific cell type (e.g., a particular cell type that does not contain a selectable marker) by causing inhibition of proliferation and/or reduction of viability of that cell type will affect other, non-similar cells in the organism to a limited extent, if at all, such that there is no significant adverse impact on the organism as a whole. It is noted that what constitutes a significant adverse impact may vary depending on the context in which it occurs. For example, what may be a significant adverse impact in a healthy organism may not be a significant adverse impact in a comprised or diseased organism.

As used herein, the term “altered” with respect to an enzyme refers to the state of the enzyme relative to an unaltered or wild type form of the enzyme. Thus, an altered enzyme is different in one or more respects from the unaltered or wild type enzyme. The difference(s) may be the result of a variety of factors, including but not limited to, changes in the amino acid composition of the enzyme, changes in the structure of the enzyme, changes in catalytic activity, changes in the binding properties, changes in the affinity of the enzyme for other proteins and compounds, changes in the inhibitor profile of the enzyme, and changes in the regulation of the enzyme or the expression of the enzyme. An alteration in the amino acid composition of an enzyme may be amino acid substitutions, deletions and/or additions relative to an unaltered or wild type enzyme. In particular embodiments herein, altered enzymes are resistant to inhibition by a condition that inhibits a form of the enzyme that is not altered in one or more of the ways that the altered enzyme is changed relative to the unaltered or wild type enzyme.

As used herein, an “unaltered” enzyme is a form of an enzyme that is inhibited by some condition to which an altered form of the enzyme is resistant. An unaltered enzyme may be a wild type form of the enzyme or may be altered relative to the corresponding wild type form. However, the unaltered form is different from the altered form in one or more aspects such that the unaltered form is inhibited at least one condition to which the altered form of the enzyme is resistant.

As used herein, the term “resistant” or “resistance” with reference to a condition refers to the state of being substantially unaffected by the condition. Thus, for example, cells resistant to a condition that inhibits proliferation of cells may be inhibited in proliferation to some degree but will not be inhibited in proliferation to any significant extent or to the extent that the proliferation of cells that are not resistant to the conditions would be inhibited.

An enzyme resistant to a condition is insensitive to the condition or has limited sensitivity to the condition such that it is substantially unaffected, for example the catalytic activity is substantially unaffected, by the condition. An enzyme is substantially unaffected by a condition if it has sufficient enzymatic activity to provide a requisite level of function of any system in which the enzyme is involved. Thus, for example, an enzyme involved in a biosynthetic pathway in a cell will be substantially unaffected by a condition if it has sufficient enzymatic or catalytic activity to provide for levels of biosynthesis of the product of the pathway required by the cell.

As used herein, the term “inhibitor-resistant” with reference to an enzyme refers to an enzyme that is not effectively inhibited by one or more conditions or agents that can inhibit another enzyme that performs the same or substantially similar function as the inhibitor-resistant enzyme. For example, enzymes from different sources, such as from different organisms, that perform the same function (e.g., catalyze the same reaction) may exhibit different sensitivities to inhibitors. Thus, although the activity of an enzyme from one organism may be inhibited by a certain condition or agent, the activity of an analogous enzyme from another organism may not be effectively inhibited by the condition or agent. The enzyme that is not effectively inhibited is referred to as inhibitor resistant with respect to the condition or agent. Similarly, altered enzymes, as described above, can be inhibitor resistant.

As used herein, “nonimmunogenic,” or “minimally immunogenic,” with reference to a composition means that the composition does not elicit a significant immune response in an organism exposed to the composition. In particular, any immune response in an organism exposed to a nonimmunogenic or minimally immunogenic composition would be insufficient to substantially reduce the amount and/or desired effect of the composition in the organism and is insufficient to induce an adverse reaction that is harmful to the organism. In the context of using the composition in an organism, the extent of an adverse reaction acceptable is dependent on the use contemplated.

As used herein, a “pharmaceutically acceptable salt” may be any salt derived from an inorganic or organic acid. The term pharmaceutically acceptable anion refers to an anion of such salts. The salt and the anion are chosen not to be biologically or otherwise undesirable. These salts may be formed with inorganic acids such as, for example, hydrochloric acid, hydrobromic acid, sulfuric acid (giving the sulfate and bisulfate salts), nitric acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluenesulfonic acid and salicyclic acid. Salts may also be derived from bases such as sodium, lithium, potassium, calcium, magnesium and ammonium as well as primary, secondary and tertiary amines.

As used herein, the terms “effective amount” or “selection effective amount” mean a dosage sufficient to provide for the selected greater proliferation and/or viability of cells containing a selectable marker relative to the proliferation and/or viability of substantially identical cells that do not contain the selectable marker.

As used herein, “heterologous” or “foreign” with reference to nucleic acids, DNA and RNA are used interchangeably and refer to nucleic acid, DNA or RNA that does not occur naturally as part of the genome in which it is present or which is found in a location(s) or in an amount in the genome that differs from that in which it occurs in nature. It is nucleic acid that has been exogenously introduced into the cell. Thus, heterologous nucleic acid is nucleic acid not normally found in the host genome in an identical context. Examples of heterologous nucleic acids include, but are not limited to, DNA that encodes a gene product or gene product(s) of interest, introduced for purposes of gene therapy or for production of an encoded protein. Other examples of heterologous DNA include, but are not limited to, DNA that encodes a selectable marker, DNA that encodes therapeutically effective substances, such as anti-cancer agents, enzymes and hormones, and DNA that encodes other types of proteins, such as antibodies.

As used herein, a “therapeutic product” or “therapeutically effective product” is a product that is encoded by heterologous nucleic acid that, upon introduction of the DNA into a host, yields a product that effectively ameliorates or eliminates the symptoms, manifestations of an inherited or acquired disease or that cures said disease.

As used herein, gene therapy involves the transfer or insertion of nucleic acid molecules into certain cells, which may be referred to as target cells, to produce specific gene products that are involved in correcting or modulating diseases or disorders. The nucleic acid is introduced into the selected target cells in a manner such that the nucleic acid is expressed and a product encoded thereby is produced. Alternatively, the nucleic acid may in some manner mediate expression of nucleic acid that encodes a therapeutic product. This product may be a therapeutic compound, which is produced in therapeutically effective amounts or at a therapeutically useful time. It may also encode a product, such as a peptide or RNA, that in some manner mediates, directly or indirectly, expression of a therapeutic product. Expression of the nucleic acid by the target cells within an organism afflicted with a disease or disorder thereby provides a way to modulate the disease or disorder. The nucleic acid encoding the therapeutic product may be modified prior to introduction into the target cell in order to enhance or otherwise alter the product or expression thereof.

For use in gene therapy, cells can be transfected in vitro, followed by introduction of the transfected cells into the body of a subject. This is often referred to as ex vivo gene therapy. Alternatively, the cells can be transfected directly in vivo within the body of a subject.

As used herein, “expression” refers to the process by which nucleic acid, e.g., DNA, is transcribed into mRNA and translated into peptides, polypeptides, or proteins. If the nucleic acid is derived from genomic DNA, expression may, if an appropriate eukaryotic host cell or organism is selected, include splicing of the mRNA.

As used herein, “vector” or “plasmid” refers to discrete elements that are used to introduce heterologous nucleic acids into cells. Typically, vectors are used to transfer heterologous nucleic acids into cells for either expression of the heterologous nucleic acid or for replication of the heterologous nucleic acid. Selection and use of such vectors and plasmids are well within the level of skill of the art.

As used herein, “transformation” or “transfection” refers to the process by which nucleic acids are introduced into cells. Transfection refers to the taking up of exogenous nucleic acid, e.g., an expression vector, by a host cell whether or not any coding sequences are in fact expressed. Numerous methods of transfection are known to the ordinarily skilled artisan. Successful transfection is generally recognized by detection of the presence of the heterologous nucleic acid within the transfected cell, such as, for example, any visualization of the heterologous nucleic acid or any indication of the operation of a vector within the host cell.

As used herein, “injection” refers to the microinjection (use of a small syringe) of nucleic acid into a cell.

As used herein, the amino acids, which occur in the various amino acid sequences appearing herein, are identified according to their well-known, three-letter or one-letter abbreviations (see Table 1). The nucleotides, which occur in the various DNA fragments, are designated with the standard single-letter designations used routinely in the art.

As used herein, amino acid residue refers to an amino acid formed upon chemical digestion (hydrolysis) of a polypeptide at its peptide linkages. The amino acid residues described herein are preferably in the “L” isomeric form. However, residues in the “D” isomeric form can be substituted for any L-amino acid residue, as long as the desired functional property is retained by the polypeptide. NH₂ refers to the free amino group present at the amino terminus of a polypeptide. COOH refers to the free carboxy group present at the carboxyl terminus of a polypeptide. In keeping with standard polypeptide nomenclature described in J. Biol. Chem., 243:3552-59 (1969) and adopted at 37 C.F.R. §§1.821-1.822, abbreviations for amino acid residues are shown in Table 1:

TABLE 1
Table of Correspondence

SYMBOL

| 1-Letter | 3-Letter | AMINO ACID |
|---|---|---|
| Y | Tyr | tyrosine |
| G | Gly | glycine |
| F | Phe | phenylalanine |
| M | Met | methionine |
| A | Ala | alanine |
| S | Ser | serine |
| I | Ile | isoleucine |
| L | Leu | leucine |
| T | Thr | threonine |
| V | Val | valine |
| P | Pro | proline |
| K | Lys | lysine |
| H | His | histidine |
| Q | Gln | glutamine |
| E | Glu | glutamic acid |
| Z | Glx | Glu and/or Gln |
| W | Trp | tryptophan |
| R | Arg | arginine |
| D | Asp | aspartic acid |
| N | Asn | asparagine |
| B | Asx | Asn and/or Asp |
| C | Cys | cysteine |
| X | Xaa | Unknown or other |

It should be noted that all amino acid residue sequences represented herein by formulae have a left to right orientation in the conventional direction of amino-terminus to carboxyl-terminus. In addition, the phrase “amino acid residue” is broadly defined to include the amino acids listed in the Table of Correspondence and modified and unusual amino acids, such as those referred to in 37 C.F.R. §§1.821-1.822, and incorporated herein by reference. Furthermore, it should be noted that a dash at the beginning or end of an amino acid residue sequence indicates a peptide bond to a further sequence of one or more amino acid residues or to an amino-terminal group such as NH₂ or to a carboxyl-terminal group such as COOH.

In a peptide or protein, suitable conservative substitutions of amino acids are known to those of skill in this art and may be made generally without altering the biological activity of the resulting molecule. Those of skill in this art recognize that, in general, single amino acid substitutions in non-essential regions of a polypeptide do not substantially alter biological activity (see, e.g., Watson et al. Molecular Biology of the Gene, 4th Edition, 1987, The Benjamin/Cummings Pub. co., p.224).

Such substitutions may be made in accordance with those set forth in TABLE 2 as follows:

TABLE 2

| Original residue | Conservative substitution |
|---|---|
| Ala (A) | Gly; Ser |
| Arg (R) | Lys |
| Asn (N) | Gln; His |
| Cys (C) | Ser |
| Gln (Q) | Asn |
| Glu (E) | Asp |
| Gly (G) | Ala; Pro |
| His (H) | Asn; Gln |
| Ile (I) | Leu; Val |
| Leu (L) | Ile; Val |
| Lys (K) | Arg; Gln; Glu |
| Met (M) | Leu; Tyr; Ile |
| Phe (F) | Met; Leu; Tyr |
| Ser (S) | Thr |
| Thr (T) | Ser |
| Trp (W) | Tyr |
| Tyr (Y) | Trp; Phe |
| Val (V) | Ile; Leu |

Other substitutions are also permissible and may be determined empirically or in accord with known conservative substitutions.

As used herein, a DNA or nucleic acid homolog refers to a nucleic acid that includes a preselected conserved nucleotide sequence, such as a sequence encoding a therapeutic polypeptide. By the term “substantially homologous” is meant having at least 80%, at least 90%, or at least 95% homology therewith or a less percentage of homology or identity and conserved biological activity or function.

The terms “homology” and “identity” are often used interchangeably. In this regard, percent homology or identity may be determined, for example, by comparing sequence information using a GAP computer program. The GAP program uses the alignment method of Needleman and Wunsch (J. Mol. Biol. 48:443 (1970), as revised by Smith and Waterman (Adv. Appl. Math. 2:482 (1981). Briefly, the GAP program defines similarity as the number of aligned symbols (i.e., nucleotides or amino acids) which are similar, divided by the total number of symbols in the shorter of the two sequences. Default parameters for the GAP program may include: (1) a unary comparison matrix (containing a value of 1 for identities and 0 for non-identities) and the weighted comparison matrix of Gribskov and Burgess, Nucl. Acids Res. 14:6745 (1986), as described by Schwartz and Dayhoff, eds., ATLAS OF PROTEIN SEQUENCE AND STRUCTURE, National Biomedical Research Foundation, pp. 353-358 (1979); (2) a penalty of 3.0 for each gap and an additional 0.10 penalty for each symbol in each gap; and (3) no penalty for end gaps.

Whether any two nucleic acid molecules have nucleotide sequences that are at least 80%, 85%, 90%, 95%, 96%, 97%, 98% or 99% “identical” can be determined using known computer algorithms such as the “FAST A” program, using for example, the default parameters as in Pearson and Lipman, Proc. Natl. Acad. Sci. USA 85:2444 (1988). Alternatively the BLAST function of the National Center for Biotechnology Information database may be used to determine identity

In general, sequences are aligned so that the highest order match is obtained. “Identity” per se has an art-recognized meaning and can be calculated using published techniques. (See, e.g.: Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991). While there exist a number of methods to measure identity between two polynucleotide or polypeptide sequences, the term “identity” is well known to skilled artisans (Carillo, H. & Lipton, D., SIAM J Applied Math 48:1073 (1988)). Methods commonly employed to determine identity or similarity between two sequences include, but are not limited to, those disclosed in Guide to Huge Computers, Martin J. Bishop, ed., Academic Press, San Diego, 1994, and Carillo, H. & Lipton, D., SIAM J Applied Math 48:1073 (1988). Methods to determine identity and similarity are codified in computer programs. Computer program methods to determine identity and similarity between two sequences include, but are not limited to, GCG program package (Devereux, J., et al., Nucleic Acids Research 12(I):387 (1984)), BLASTP, BLASTN, FASTA (Atschul, S. F., et al., J Molec Biol 215:403 (1990)).

Therefore, as used herein, the term “identity” represents a comparison between a test and a reference polypeptide or polynucleotide. For example, a test polypeptide may be defined as any polypeptide that is 90% or more identical to a reference polypeptide.

As used herein, the term at least “90% identical to” refers to percent identities from 90 to 99.99 relative to the reference polypeptides. Identity at a level of 90% or more is indicative of the fact that, assuming for exemplification purposes a test and reference polynucleotide length of 100 amino acids are compared. No more than 10% (i.e., 10 out of 100) amino acids in the test polypeptide differs from that of the reference polypeptides. Similar comparisons may be made between a test and reference polynucleotides. Such differences may be represented as point mutations randomly distributed over the entire length of an amino acid sequence or they may be clustered in one or more locations of varying length up to the maximum allowable, e.g. 10/100 amino acid difference (approximately 90% identity). Differences are defined as nucleic acid or amino acid substitutions, or deletions.

As used herein, “at a position corresponding to” refers to a position of interest (i.e., base number or residue number) in a nucleic acid molecule or protein relative to the position in another reference nucleic acid molecule or protein. Corresponding positions can be determined by comparing and aligning sequences to maximize the number of matching nucleotides or residues, for example, such that identity between the sequences is greater than 95%, greater than 96%, greater than 97%, greater than 98% or greater than 99%. The position of interest is then given the number assigned in the reference nucleic acid molecule. For example, it is shown herein that a particular alteration in human inosine monophosphate dehdrogenase (IMPDH) occurs at amino acid residue position 333 of SEQ. ID. No. 2. To identify the corresponding amino acid in another isolate, the sequences are aligned and then the position that lines up with 333 is identified. The same process may be applied to identify a nucleotide in a nucleotide sequence that corresponds to a particular nucleotide position in another nucleotide sequence. Since various alleles or isolates may be of different length, the position designated, for example, amino acid 333 may not be amino acid 333, but instead is at a position that “corresponds” to the position in the reference sequence.

As used herein, all assays and procedures, such as hybridization reactions and antibody-antigen reactions, unless otherwise specified, are conducted under conditions recognized by those of skill in the art as standard conditions.

A. Selection Systems Using Purine Biosynthesis Pathway Enzymes as a Selectable Marker

The selection systems disclosed herein have a variety of uses, including, but not limited to, identifying, isolating, differentially affecting and/or providing a selective advantage for proliferation of certain cells that have been genetically modified. Particular selection systems are based in the pairing of a selectable marker with selection agents that provide conditions under which cells containing the selectable marker will exhibit greater proliferation and/or viability than cells that do not contain the selectable marker. Selectable markers used in such selection systems are inhibitor-resistant or altered enzymes involved in the synthesis of nucleotides in cells. Selection agents used in the selection systems are compositions and/or conditions which inhibit sensitive (i.e., non-resistant or unaltered) forms of the enzymes in cells such that at least certain cells exposed to the inhibitor under certain conditions exhibit reduced proliferation and/or viability relative to the amount of proliferation and/or viability of the cells in the absence of the inhibitor.

Exemplary selectable markers which may be used in selection systems as described herein include inhibitor-resistant or altered forms of enzymes of the purine and pyrimidine biosynthesis pathways. Such enzymes include, but are not limited to, inosine monophosphate dehydrogenase (IMPDH), dihydroorotate dehydrogenase (DHODH), ribose phosphate pyrophosphokinase, amidophosphoribosyltransferase, GAR synthetase, GAR transformylase, FGAM synthetase, AIR synthetase, AIR carboxylase, SAICAR synthetase, adenylosuccinate synthase, adenylosuccinate lyase, AICAR transformylase, IMP cyclohydrolase, GMP synthase, carbamoyl phosphate synthetase II, aspartate transcarbamylase, dihydroorotase, orotate phosphoribosyl transferase, OMP decarboxylase, CTP synthase, thymidylate synthase, and ribonucleotide reductase. Inhibitors of these enzymes, which may be useful as selection agents in systems using inhibitor-resistant forms of these enzymes as selectable markers, are known and can be identified by those of skill in the art. Included among such inhibitors are: mycophenolic acid and derivatives thereof, tiazofurin, ribavirin, EICAR, mizoribine, pyridazines, cinchoninic acid derivatives, naphthoquinone derivatives, isoxazole derivatives, acivicin, 6-mercaptopurine (an inhibitor of adenylosuccinate synthase and adenylosuccinate lyase) and folate anti-metabolites such as 5,10-dideaza-5,6,7,8-tetrahydrofolic acid (DDATHF, lometrexol; inhibitors of the folate requiring enzymes GAR transformylase and AICAR transformylase) [see e.g., Weber et al. (1991) Adv. Enzyme Regul. 31:45-67]. Enzymes resistant to such inhibitors are known or can be generated, for example, using methods provided herein or known to those of skill in the art.

In a particular embodiment of the selection systems provided herein, an inhibitor-resistant or altered enzyme of a purine biosynthesis pathway is used as a selectable marker. The may be a rate-limiting enzyme of de novo purine nucleotide biosynthesis. For example, in some selection systems, an inhibitor-resistant or altered inosine monophosphate dehydrogenase (IMPDH) enzyme (particularly a human IMPDH), which serves as a selectable marker, is used to confer on cells in which it is expressed resistance to an inhibitor of IMPDH (i.e., a selection agent). If the selectable marker is an altered IMPDH, the alteration in the IMPDH is such that the enzyme is no longer effectively inhibited by the selection agent.

1. Inosine Monophosphate Dehydrogenase (IMPDH)

Inosine monophosphate dehydrogenase (IMPDH) is an enzyme involved in the de novo synthesis of guanine nucleotides and is ubiquitous in eukaryotes, bacteria and protozoa. The de novo pathway begins with the conversion of 5-phosphoribosyl-1-pyrophosphate (PRPP) to inosine monophosphate (IMP). IMPDH catalyzes the NAD-dependent oxidation of inosine monophosphate (IMP) to xanthine monophosphate (XMP), which is rapidly converted to guanosine monophosphate (GMP). Guanosine triphosphate, GTP, which is used in nucleic acid synthesis, is produced from GMP.

IMPDH is the rate-limiting enzyme in the de novo synthesis of guanine nucleotides. Many studies have investigated the mechanism of the reaction catalyzed by IMPDH. In one proposed mechanism, the enzyme follows an ordered Bi-Bi reaction sequence of substrate and cofactor binding and product release. First, IMP binds to IMPDH. This is followed by the binding of the cofactor NAD. The reduced cofactor, NADH, is then released from the product, followed by the product, XMP [see, e.g., Carr et al. (1993) J. Biol. Chem. 268:27286-27290 and Holmes et al. (1974) Biochim. Biophys. Acta 364:209-217]. Another study [Wang and Hedstrom (1997) Biochemistry 36:8479-8473] has demonstrated random binding of substrates while product release occurs in an ordered sequence.

A salvage pathway for guanine biosynthesis, which works by recycling purine bases, is also available in normal mammalian cells, in addition to the de novo pathway. The salvage pathway involves PRPP and guanine, which are converted to GMP via hypoxanthine-guanine phosphoribosyltransferase (HGPRT), and does not utilize IMPDH. In the salvage pathway, GMP can be generated from guanosine or guanine. Proliferating lymphocytes depend primarily on the de novo pathway for purine biosynthesis while most other cell types, including, for example, fibroblasts, smooth muscle cells, endothelial cells and intestinal epithelial cells, are able to use both the de novo and salvage pathways. In some cancer cells, such as rat hepatoma and sarcoma, human primary colon carcinoma and human chronic granulocytic leukemia in blast crisis, the activity of enzymes of the purine salvage pathway may be higher than those of the rate-limiting enzymes of de novo biosynthesis. Also, the metabolic flux through the salvage pathway in these cells has been shown to increase after treatment with inhibitors of de novo guanylate biosynthesis (Weber et al. (1991) Adv. Enzyme. Regul. 31:45-67).

Distinct genes encoding two isoforms of human IMPDH (i.e., IMPDH type I and IMPDH type II) have been reported [see, e.g., Glesne et al. (1993) Genomics 16:274-277 and Gu et al. (1994) Genomics 24:179-181]. Complementary DNA encoding the two isoforms has been isolated [see, e.g., Natsumeda et al. (1990) J. Biol. Chem. 265:5292-5295, Collart and Huberman (1988) J. Biol. Chem. 263:15769-15772, and U.S. Pat. No. 5,665,583]. Each isoform has 514 amino acids and they share 84% sequence identity. The human IMPDH type II gene, including its 5′ flanking region, has been cloned and characterized [see Zimmermann et al. (1994) J. Biol. Chem. 270:6808-6814].

IMPDH activity is closely positively correlated with cell proliferation. Elevated IMPDH activity has been observed with increased proliferation of both normal and malignant tissues and cells, as well as malignant transformation [see, e.g., Jackson and Weber (1975) Nature 256:331-333 and Natsumeda et al. (1988) Cancer Res. 48:507-511 ]. Inhibitors of IMPDH activity have been shown to inhibit cell proliferation and induce cellular differentiation [see, e.g., Kiguchi et al. (1990) Cell Growth Differ. 1:259-270]. Regulation of IMPDH activities during cellular growth, transformation and differentiation has been largely attributed to changes in the expression of the IMPDH type II gene (the type II gene is upregulated in proliferating cells and highly downregulated (90%) in terminally differentiated cells), whereas the type I gene remains constitutively expressed [see, e.g., Nagai et al. (1992) Cancer Res. 52:258-261]. A possible explanation for the presence of two isoforms of IMPDH is a need for a small amount of de novo GTP synthesis in differentiated cells for “housekeeping” purposes and RNA synthesis, while DNA synthesis occurring in proliferating cells requires a higher rate of GTP production [see, e.g., Yalowitz and Jayaram (2000) Anticancer Res. 20:2329-2338].

2. Inhibitors of IMPDH

Inhibitors of IMPDH that may be used as selection agents in systems employing an inhibitor-resistant or altered IMPDH as a selectable marker typically provide for a level of inhibition of IMPDH in cells such that at least certain cells exposed to the inhibitor under certain conditions exhibit reduced proliferation and/or viability relative to the amount of proliferation and/or viability of the cells in the absence of the inhibitor. At minimum, cells that may exhibit reduced proliferation and/or viability when exposed to the IMPDH inhibitor are those that do not contain an inhibitor-resistant or altered IMPDH that confers resistance to conditions that include the presence of the inhibitor, but that are otherwise substantially identical to cells that contain the inhibitor-resistant or altered IMPDH. Inhibitors of IMPDH that may be used as selection agents in selection systems provided herein generally are ineffective at substantially inhibiting an inhibitor-resistant or altered IMPDH that may be used as a selectable marker.

The ability of IMPDH inhibitors to inhibit cell proliferation and/or viability may be determined by a variety of methods. For example, generally, cell proliferation may be determined in the absence of and in the presence of increasing concentrations of inhibitor by measuring [³H]thymidine incorporation by cells as an assessment of DNA synthesis using standard methods known to those of skill in the art. Inhibition of cell proliferation by IMPDH inhibitors may also be assessed in in vitro and in vivo cell proliferation assays described herein (see, for example, Examples 6 and 7) and/or known to those of skill in the art. For example, the procedure of Greaves et al. [(1974) Nature 248:698-701], and modified versions thereof (see, e.g., U.S. Pat. No. 5,380,879), may be used to evaluate the in vitro effect of a compound on the responses of human peripheral blood lymphocytes to T- and B-cell mitogens. The ability of a compound to inhibit the proliferation of mouse mammary carcinoma cells (EMT6 cells) is a further assay that may be used to evaluate the effects of IMPDH inhibitors on cell proliferation [see, e.g., Franklin et al. (1999) Biochem. Pharmacol. 58:867-876]. The hemolytic plaque forming cell assay procedure of Jerne et al. [Cellbound Antibodies, Amos and Kaprowski, eds. (Wistar Institute Press, Philadelphia, 1963) p. 109], and modified versions thereof (see, e.g., U.S. Pat. No. 5,380,879), may be used, for example, to evaluate the in vivo immunosuppressive activity of a compound, which is an indication of the effect of the compound on the ability of immune system cells to proliferate and generate a response to antigen. Another assay for the immunosuppressive activity of a compound utilizes a model of DTH in which the effect of the compound on the response of mice that had been immunized with ovalbumin to a challenge by intraplantar injection of ovalbumin is assessed by measuring footpad thickness as an indicator of the degree of inflammation [see, e.g., Franklin et al. (1999) Biochem. Pharmacol. 58:867-876].

Methods of assessing cell viability are known to those of skill in the art and include, but are not limited to, methods using indicators of cell function. For example, evidence of cell viability may also be based on assessments of basal metabolism or cell proliferation. The demonstration of synthesis of cell products (e.g., uptake of labeled amino acids into newly synthesized protein) is also indicative of cell viability.

Cell membrane integrity is commonly used to indicate cell viability. For example, the ability of cells to exclude certain dyes, such as trypan blue, which accumulate in dead cells may be used to assess cell viability. One such procedure employing trypan blue for counting and calculating the number of live cells, involves loading a hemacytometer with a mixture of cell suspension and trypan blue. Microscopic examination of the loaded hemacytometer is used to count viable cells (unstained cells) and dead cells (blue-stained cells), and the total number of viable cells in the suspension and percentage of viable cells in the population may be calculated (see, e.g., EXAMPLE 1). Cell viability may also be assessed by using fluorogenic reagents that distinguish live and dead cells (see, e.g., U.S. Pat. No. 5,314,805).

IMPDH inhibitors include, but are not limited to, mycophenolic acid (MPA), tiazofurin (2-,β-D-ribofuranosylthiazole-4-carboxamide), ribavirin, 5-ethynyl-1-,β-D-ribofuranosylimidazole-4-carboxamide (EICAR), mizoribine (Bredinin), selenazole-4-carboxamide adenine dinucleotide (SAD), VX-497 and pyridazines [see, e.g., Pankiewicz (1999) Exp. Opin. Ther. Patents 9:55-65].

a. Mycophenolic Acid

Mycophenolic acid (MPA) is a noncompetitive, reversible inhibitor of the enzyme IMPDH [see, e.g., Sievers et al. (1997) Pharmacotherapy 17:1178-1197] with antineoplastic, antitumor, antibacterial, antifungal, antiviral and immunosuppressive properties [see, e.g., Hood and Zarembski (1997) Am. J. Health-Syst. Pharm. 54:285-294]. MPA has an approximately 5-fold higher binding affinity for human IMPDH type II over human IMPDH type I [see Carr et al. (1993) J. Biol. Chem. 268:27286-27290 and Natsumeda et al. (1993) Ann. N.Y. Acad. Sci. 696:88-93]. MPA shows uncompetitive inhibition of human IMPDH with a Kᵢ of 156 nM versus NAD [Yalowitz and Jayaram (2000) Anticancer Res. 20:2329-23381. Mycophenolic acid affinity varies among microbial and mammalian IMPDH as is evident from differences in Kᵢ values: 22 nM for human IMPDH, 500 nM for Bacillus subtilis IMPDH, 20 μM for Escherichia coli IMPDH and 14 μM for Tritrichomonas foetus IMPDH [Farazi et al. (1997) J. Biol. Chem. 272:961-965 ].

Mycophenolate mofetil (MMF) is an organic synthetic derivative of the natural fermentation product MPA [see, e.g., Nelson et al. (1990) J. Med. Chem. 33:833-838 and Lee et al. (1990) Pharm. Res. 7:161-166]. Mycophenolate mofetil is a morpholinoethylester prodrug of MPA which is rapidly hydrolyzed to MPA after oral administration. MMF has also been used in the treatment of allograft rejection in solid organ transplant patients [see, e.g., The Mycophenolate Mofetil Renal Refractory Rejection Study Group (1996) Transplantation 61:722-729] and has been investigated in the treatment of autoimmune disorders such as psoriasis and rheumatoid arthritis [see, e.g., Sievers et al. (1997) Pharmacotherapy 17:1178-1197].

Derivatives and analogs of MPA are also useful as selection agents in the systems provided herein. Numerous MPA derivatives and analogs have been reported, including, but not limited to, those described in U.S. Pat. Nos. 4,686,234; 4,725,622; 4,727,069; 4,748,173; 4,753,935; 4,786,637; 4,808,592; 4,861,776; 4,868,153; 4,948,793; 4,952,579; 4,959,387; 4,922,467; 5,247,083; 5,380,879 (including derivatives of MPA in which the 4-hydroxy group has been replaced by amino substituents); and 5,444,072 (including derivatives of MPA in which the 6-methoxy group has been replaced with other substituents) and in PCT International Patent Application Nos. PCT/US92/09932 (WO94/12184), PCT/US93/06410 (WO94/01105).

b. Ribavirin

The monophosphate form of ribavirin [see, e.g., Perigaud et al. (1992) Nucleosides Nucleotides 11:903-945] is a substrate mimic of IMP and acts as a competitive inhibitor of IMPDH [see Christopherson and Lyons (1990) Med. Res. Rev. 10:505-548].

C. Tiazofurin

Tiazofurin is phosphorylated intracellularly to its monophosphate form, which is then converted to the active metabolite thiazole-4-carboxamide adenine dinucleotide or TAD. This NAD+analog, containing tiazofurin instead of the nicotinamide riboside moiety, inhibits IMPDH activity through competition for the NAD⁺cofactor-binding site of the enzyme [see, e.g., Lui et al (1984) J. Biol. Chem. 259:5078-50821. TAD inhibits IMPDH with a Kᵢ of 0.075 μM against NAD [see Yamada et al. (1989) Leuk. Res. 13:179-184 ].

d. EICAR

EICAR (5-ethynyl-1-β-D-ribofuranosylimidazole-4-carboxamide) binds to IMPDH in the IMP binding pocket of the molecule and inhibits IMPDH by covalently modifying the active site cysteine. A similar dinucleotide metabolite (i.e., EAD) is also formed from the monophosphorylated form of EICAR [see, e.g., Balzarini et al. (1993) J. Biol. Chem. 268:24591-24598].

e. Mizoribine (Bredinin)

Mizoribine is a nucleoside analog that can be phosphorylated intracelluarly to the 5′-monophosphate which then inhibits IMPDH competitively [see, e.g., Kusumi et al. (1988). Cell. Biochem. Function 7:201-204].

f. Selenazole-4-carboxamide adenine dinucleotide (SAD)

SAD is a NAD⁺mimic and is an analog of the tiazofurin metabolite TAD [Cooney et al. (1982) Biochem. Pharmacol. 31:2133-2136].

g. Pyridazines

Pyridazines and derivatives thereof, such as those reported by Franklin et al. [(1999) Biochem. Pharmacol. 58:867-876], are non-nucleoside, uncompetitive inhibitors of IMPDH, particularly IMPDH type II, which also have significant immunosuppressive activity.

h. VX-497

VX-497 is a nanomolar, uncompetitive inhibitor of both isoforms of IMPDH (Ki=7 nM against type I and 10 nM against type II). It inhibits B cell and T cell proliferation with an IC₅₀ of approximately 100 nM [see Sintchak and Nimmesgern (2000) Immunopharmacology 47:163-184].

i. Other

Additional inhibitors of IMPDH are provided, for example, in U.S. Pat. Nos. 5,569,650, 5,658,890, 5,700,786, 5,807,876, 5,932,600 and 6,054,472 and PCT International Patent Application Nos. PCT/US97/06623 (WO97/40028), PCT/US98/04932 (WO98/40381) and PCT/US99/09005 (WO99/55663).

j. Methods of Identifying Inhibitors of IMPDH

Inhibitors of IMPDH may also be identified in a variety of screening assays. For example, candidate inhibitory compounds may be tested for ability to inhibit IMPDH activity assessed in an HPLC assay or spectrophotometric assay. Thus, IMPDH activity may be determined by monitoring NADH production as a change in absorbance spectrophotometrically of a reaction mixture containing IMP, IMPDH and NAD [see, e.g., Farazi et al. (1997) J. Biol. Chem. 272:961-965 and Anderson and Sartorelli (1968) J. Biol. Chem. 243:4762-4768]. IMPDH activity may also be determined, for example, by using high pressure liquid chromatography on a C18 column with ion pairing reagents to separate IMP, NAD, XMP and NADH. The extent of reaction is determined from the resulting peak areas (see, e.g., U.S. Pat. No. 6,054,472 and Example 7). This assay is particularly useful in determining the inhibition profiles of compounds which have significant absorbance in the UV-visible region between 290 and 340 nm.

A high throughput screening assay for inhibitors of IMPDH is based on quantitative determination of the NADH formed in the IMPDH-catalyzed reaction by coupling the diaphorase-catalyzed oxidation of NADH to the reduction of MTT [see, e.g., Franklin et al. (1999) Biochem. PharmacoL. 58:867-876]. The optical density of the reduced MTT generated in the reaction is monitored at 540 nm.

In another method for identifying inhibitors of IMPDH [see PCT International Patent Application No. PCT/IB98/02109 (WO99/33996)], organisms auxotrophic for guanine (due to a defective IMPDH gene which fails to produce functional IMPDH enzyme), for example, Escherichia coli strain H712 [see Nijkamp and De Haan (1967) Biochim. Biophys. Acta 145:31-40], that have been transformed with DNA encoding an IMPDH enzyme of interest are exposed to candidate inhibitors. IMPDH inhibitors are identified by an ability to inhibit transformed cell proliferation. Specificity of the inhibitor for IMPDH is evaluated by supplementing the transformed cell culture media with guanine nucleotide precursors to correct the defect. Guanine auxotrophs with a specific IMPDH defect that are suitable for use in this screening system have been reported for both prokaryotic [Nijkamp and De Haan (1967) Biochim. Biophys. Acta 145:31-40 and Freese et al. (1979) J. Gen. Microbiol. 115:193-205] and eukaryotic [Greer and Wellman (1980) Can. J. Microbiol. 26:1412-1415] organisms.

3. Inhibitor-Resistant IMPDH Enzymes

Inhibitor-resistant IMPDH enzymes useful as selectable markers in particular selection systems described herein provide in cells in which they are expressed increased resistance to inhibition of cell proliferation and/or reduction of cell viability by an inhibitor of an IMPDH relative to the resistance of cells that do not express the inhibitor-resistant IMPDH. Resistance to inhibition of cell proliferation and/or reduction of cell viability can be assessed using methods described herein or known in the art. For example, cells can be grown in media containing incrementally increasing concentrations of an IMPDH inhibitor, and cell number at each concentration may be determined using standard techniques such as a Coulter counter [see, e.g., Hodges et al. (1989) J. Biol. Chem. 264:18137-18141]. Viable cell numbers may also be determined at each concentration using methods described herein and known to those of skill in the art.

Cell proliferation may be determined, for example, by measuring [³H]thymidine incorporation by cells as an assessment of DNA synthesis using standard methods known to those of skill in the art. The results can be evaluated in a number of ways. For example, the concentration of inhibitor that results in a 50% reduction of cell proliferation over a certain time period, i.e., the IC₅₀ value, can be determined. Results may also be evaluated as a plot of the reciprocal fraction of the mean doubling time of inhibitor-treated cultures over a particular growth period compared to the doubling time in the absence of inhibitor versus inhibitor concentration.

Similarly, using methods described herein and known to those of skill in the art, viable cell numbers may be calculated in the presence of increasing concentrations of inhibitor. The concentration of inhibitor that results in a 50% reduction in viable cells, i.e., the IC₅₀, can be determined.

Cells expressing an inhibitor-resistant or altered IMPDH enzyme will exhibit greater proliferation and/or viability at increasing concentrations of inhibitor than cells that express a sensitive or unaltered IMPDH enzyme. Therefore, a higher concentration of inhibitor is required to achieve a the same level of reduction in cell proliferation and/or viability in cells expressing an inhibitor-resistant IMPDH as compared to cells expressing a sensitive IMPDH. Thus, cells expressing an inhibitor-resistant IMPDH enzyme will exhibit increased resistance to inhibition of cell proliferation and/or reduction of cell viability by an IMPDH inhibitor. Some inhibitor-resistant IMPDH enzymes for use as selectable markers provide in cells in which they are expressed about 2- to 10,000-fold or greater (typically about 5- to 2500-fold or 10- to 500-fold) increased resistance to inhibition of cell proliferation and/or reduction of cell viability by an inhibitor of an IMPDH. This can be expressed, for example, as a 2- to 10,000-fold increase in the IC₅₀ value for growth inhibition and/or viability reduction for the inhibitor of IMPDH.

Increased resistance to inhibition of cellular proliferation and/or reduction of cell viability can be the result of increased Kᵢ of an inhibitor-resistant IMPDH (as compared to a inhibitor-sensitive or unaltered or wild-type IMPDH) or the combination of increased Kᵢ and increased expression (i.e., resulting in increased protein levels) of an inhibitor-resistant IMPDH in resistant cells relative to the amount of expression of an inhibitor-sensitive IMPDH in non-resistant cells. It is possible for increased resistance to inhibition of cell proliferation and/or reduction of cell viability to be achieved by virtue of the reduced sensitivity of the inhibitor-resistant IMPDH for an inhibitor such that substantially increased expression is not required to effect selection of cells containing the inhibitor-resistant IMPDH. Thus, selection of genetically modified cells is achieved in these selection systems through introduction and expression of as few as about 1 to about 10, or about 2 to about 10 or about 1 to about 5 or about 2 to about 5 or about 2 copies or 1 copy of a nucleic acid encoding an inhibitor-resistant IMPDH into a cell. Thus, increased resistance to inhibition of cell proliferation and/or reduction of cell viability may be achieved by virtue of the reduced sensitivity of the inhibitor-resistant IMPDH to an inhibitor without depending on increased levels of expression of the inhibitor-resistant IMPDH.

Indications of increased expression or gene amplification include increased mRNA and/or protein levels, increased enzyme activity when cells are cultured in the presence of inhibitor and decreased enzyme activity when cells are cultured in the absence of inhibitor. Changes in the kinetic or inhibition parameters (e.g., the Kₘ and Kᵢ values) of an inhibitor-resistant or altered IMPDH relative to those of an inhibitor-sensitive or unaltered IMPDH are reflective of an inherent reduced sensitivity of the enzyme irrespective of the amount of enzyme that may be present.

Generally, such IMPDH enzymes which provide for increased resistance to inhibition of cell proliferation and/or reduction of cell viability by an IMPDH inhibitor are not effectively inhibited by one or more conditions that can inhibit an IMPDH. Such enzymes, which are generally referred to as inhibitor-resistant, may be from an organism (e.g., a microbe) that expresses an IMPDH that has an inhibitor sensitivity that differs from that of IMPDH enzymes from other organisms (e.g., a mammal) or may be an IMPDH that is altered relative to wild-type IMPDH such that an inhibitor of wild-type IMPDH is no longer an effective inhibitor of the enzyme. Although the activity of the altered IMPDH enzyme may differ from that of the wild-type enzyme, it must be sufficient to effectively catalyze the oxidation of IMP to XMP and thereby maintain cellular guanine deoxy- and ribonucleotide pools required for DNA and RNA biosynthesis.

In particular embodiments of the selection systems using inhibitor-resistant, including altered, IMPDH as a selectable marker, the resistant IMPDH enzymes are nonimmunogenic or minimally immunogenic with respect to host organisms in which they may be used. Immunogenicity of IMPDH enzymes may be evaluated using a variety of assays such as those described herein and known in the art.

a. Catalytic Activity and Inhibitor Sensitivity of Inhibitor-Resistant IMPDH Enzymes

Inhibitor-resistant enzymes are insensitive to or have reduced sensitivity to one or more inhibitors of IMPDH enzymes. The catalytic activity of altered IMPDH enzymes is substantially similar to or at least not significantly reduced relative to the catalytic activity of wild-type IMPDH enzymes.

The activity and inhibitor sensitivity of inhibitor-resistant or altered IMPDH enzymes may be evaluated using assays as described herein or any assay known in the art for measuring IMPDH activity. One such method involves monitoring NADH production catalyzed by the altered IMPDH enzyme, for example, by measuring a change in absorbance spectrophotometrically, in the presence of IMP and NAD [see, e.g., Farazi et al. (1997) J. Biol. Chem. 272:961-965 and Anderson and Sartorelli (1968) J. Biol. Chem. 243:4762-4768]. Generally, reaction solutions (0.5-1.0 ml) may contain 0.1M Tris HCI, pH 8.0; 0.1M KCl; 3.0 mM EDTA; 100 μg/ml BSA; 0.05 mM IMP; 0.10 mM NAD; 10% DMSO; and 5-15 nM altered IMPDH enzyme, and the reactions are conducted at 40° C. and initiated by addition of the altered IMPDH enzyme. In an adaptation of the method of Magasanik et al. [(1957) J. Biol. Chem. 226:339], an alternative reaction mixture is as above but contains 0.1 M IMP and 15-50 nM altered IMPDH enzyme and additionally contains 2 mM DTT; the reaction is performed at 37° C. and started by adding NAD to a final concentration of 0.1 M (see U.S. Pat. No. 6,054,472).

The reaction mixture may be prepared in disposable methacrylicplastic microcuvets (“UV-transparent” plastic, 1 cm pathlength, 1.5 ml capacity). The reactions are monitored at 340 nm for 10 minutes in a UV/VIS spectrophotometer, and rate data are collected. Affinity for the inhibitor may also be determined by including varying concentrations of inhibitor and NAD in the reactions. For example, the inhibitor may be dissolved in DMSO to a final concentration of 20 mM and added to the initial assay mixture for preincubation with the enzyme at a final volume of 2-5% (v/v). The reaction is started by addition of NAD, and the initial rates measured. Kᵢ determinations may be made by measuring the initial velocities in the presence of varying amounts of inhibitor and fitting the data using the tight-binding equations of Henderson [(1972) Biochem. J. 127:321] (see U.S. Pat. No. 6,054,472).

The data obtained from IMPDH activity assays in the presence and absence of inhibitor may be used to determine the Kₘ and k_{cat} values of the inhibitor-resistant or altered IMPDH enzyme and the Kᵢ for the inhibitor [see, e.g., Farazi et al. (1997) J. Biol. Chem. 272:961-965]. The Kₘ values for type I and type II IMPDHs are typically in the range of approximately 18 and 9 μM, respectively, for IMP, and 46 and 32 μM, respectively for NAD [see Natsumeda and Carr (1993) Ann. N.Y. Acad. Sci. 696:88-93]. The Kᵢ values for type I and II IMPDHs are in the range of approximately 102 and 90 μM, respectively, for NADH, and 80 and 94 μM, respectively, for XMP [see Natsumeda and Carr (1993) Ann. N.Y. Acad. Sci. 696:88-93]. Some inhibitor-resistant or altered IMPDH enzymes may have a Kᵢ for inhibitor that is in the range of about 1.5-fold to about 2500 (or more)-fold or about 3-fold to 2500-fold or about 3-fold to 500-fold greater than the Kᵢ of the inhibitor-sensitive or wild-type enzyme for the inhibitor. Some inhibitor-resistant or altered IMPDH enzymes are less sensitive to inhibitor than inhibitor-sensitive or wild-type enzymes by a factor of about 1.5-fold to 2500 (or more)-fold or about 3-fold to 2500-fold or about 3-fold to 500-fold or about 5-fold to 200-fold. Sensitivity can be expressed, for example, as the IC₅₀ (i.e., the inhibitor concentration that effects half-maximal inhibition, which will be greater for less sensitive enzymes) value of an enzyme for an inhibitor, as can be determined, for example, by plotting the fractional velocity of the enzyme (vᵢ/vₒ) versus inhibitor concentration [see, e.g., Davis et al. (1997) Biochem. Pharmacol. 54(4):459-465]. Some altered IMPDH enzymes may have a Kₘ for IMP and/or NAD that is at least similar to that of the wild-type enzyme or within a range of about 1.5-fold to about 5-fold or about 1.5-fold to about 3-fold of the inhibitor-sensitive or wild-type enzyme. Some inhibitor-resistant or altered IMPDH enzymes may have a k_{cat} value that is at least similar to that of the inhibitor-sensitive or wild-type enzyme or within a range of 0.1- to 0.9-fold of that of the inhibitor-sensitive or wild-type enzyme.

b. Generation of Altered IMPDH Enzymes

Altered IMPDH enzymes may be obtained by a variety of means. For example, altered IMPDH-encoding DNA may be generated by random mutagenesis of the cDNA encoding a wild-type IMPDH enzyme followed by selection of cells containing the mutagenized cDNA for the ability to grow in the presence of an inhibitor of wild-type IMPDH. Site-directed mutagenesis of a wild-type IMPDH-encoding DNA may also be used to obtain an altered IMPDH-encoding DNA.

(1) Random Mutagenesis

One method of generating altered IMPDH-encoding DNA involves transferring a complementary DNA encoding a wild-type IMPDH enzyme (such as that having a coding sequence substantially similar to that set forth as nucleotides 48 to 1589 in SEQ. ID. NO. 1) into a mutator strain of Escherichia coli, such as mutD5 E. coli strain NR9072 [see, e.g., Schaaper (1988) Proc. Natl. Acad. Sci. U.S.A. 85:8126-8130]. Spontaneous mutations of the wild-type IMPDH cDNA will occur when it is contained on a plasmid that is introduced into mutator strains which have defects in processes such as exonucleolytic proofreading during DNA replication and mutHLS-encoded mismatch correction.

Plasmid containing the IMPDH-encoding cDNA is then isolated from a culture of the transformed mutD5 E. coli and used to transform an E. coli strain that is defective in IMPDH activity, e.g., E. coli strain H712. E. coli strain H712 carries a partial deletion in guaB, the gene encoding IMPDH [see Nijkamp and De Haan (1967) Biochim. Biophys. Acta 145:31-40]. A wild-type human IMPDH type II-encoding cDNA can complement the guaB deficiency of H712 cells [see, e.g., Farazi et al. (1997) J. Biol. Chem. 272:961-965]. Inhibitor-resistant clones of the H712 cells transformed with the library of randomly mutagenized IMPDH-encoding cDNA may be selected by growth on minimal medium containing the inhibitor.

The inhibitor-resistant clones are then characterized to determine if there is an alteration in the IMPDH-encoding cDNA that reduces or eliminates its sensitivity to the inhibitor yet does not significantly reduce the catalytic activity of the enzyme. For example, IMPDH enzyme can be purified from cell lysates using an IMP affinity column and then assayed for activity using assays as described herein or any assay known in the art for measuring IMPDH activity.

(2) Site-Directed Mutagenesis

Altered IMPDH-encoding DNA may also be generated by site-directed mutagenesis. One method for performing site-directed mutagenesis employs a nucleic acid amplification mutagenesis technique [see, e.g., Innis et al. (1990) in PCR Protocols: A Guide to Methods and Applications]. This technique requires two rounds of PCR amplification and four oligonucleotide primers, two inside primers and two outside primers. The two inside primers are of opposite orientation and contain a region of complementarity (greater than or equal to 13 bases), with at least one primer incorporating the desired mutation. The outside primers are complementary to the template and define the 5′ and 3′ limits of the fragment to be amplified. In the first round of PCR, a plasmid containing an IMPDH-encoding DNA is used as a template for two separate reactions in which (1) a forward outside primer is combined with a reverse inside primer, and (2) a reverse outside primer is combined with a forward inside primer. Following purification of the two PCR products, a second round of PCR is initiated combining the first two PCR products. The fragments from the first round of PCR contain a region of complementarity as defined by the original inside primer pair and, therefore, serve as the template. The outside primers are used to amplify the full-length fragment.

To reduce the risk of base misincorporation, additional unique restriction sites can be incorporated into the IMPDH-encoding DNA template at intervals of approximately 300 bp to minimize the size of the amplified DNA fragment. Restriction sites can be introduced using general PCR mutagenesis techniques described above. The IMPDH plasmid incorporating additional restriction sites is then used as the template for the two separate reactions in the first round of PCR as described above. In the first reaction, the forward primer containing the mutation is combined with a reverse primer containing the nearest unique restriction site. In the second reaction, the reverse primer containing the mutation is combined with a forward primer containing the nearest restriction site. Following purification, the two PCR products are combined in a second round of PCR and amplified with the outside primers containing the nearest restriction sites. This fragment is then digested by the nearest unique restriction enzymes and subcloned into a plasmid from which the corresponding wild-type fragment has been removed. Incorporation of the desired mutation and absence of nonspecific mutations can be confirmed by fluorescent cycle sequencing of each plasmid.

IMPDH mutant plasmid can then be transformed into a purine auzotrophic strain of E. coli, H712 [see Nijkamp and De Haan (1967) Biochim. Biophys. Acta 145:31-40], which lacks a complete endogenous gene for the bacterial IMPDH enzyme. The IMPDH mutants are then characterized to determine if the mutation reduces or eliminates its sensitivity to the inhibitor yet does not significantly reduce the catalytic activity of the enzyme. For example, cell lysate can be use directly or IMPDH enzyme can be purified and then assayed for activity using assays as described herein or any assay known in the art for measuring IMPDH activity.

C. Exemplary Inhibitor-Resistant IMPDH Enzymes

Inhibitor-resistant IMPDH enzymes include microbial enzymes, e.g., T. foetus IMPDH [see Digits et al. (1999) Biochem. 38:15388-97; see also amino acid sequence set forth in SEQ ID NO. 14] which is poorly inhibited by MPA, and altered IMPDH enzymes. Altered IMPDH enzymes include enzymes having alterations relative to the amino acid sequences set forth in SEQ. ID. NOS. 2, 18 and 30 that impart resistance on the enzyme to an IMPDH inhibitor. Included among such altered IMPDH enzymes are those that contain one or more alterations in the sequence of amino acids 330-351, 330-441, 330-400, 330-355 or 333-351 of SEQ. ID. NOS. 2, 18 and 30. Particular alterations of the amino acid sequence set forth in SEQ. ID. NO. 2 include one or more of the following: sequences in which amino acid 333 is other than threonine, amino acid 351 is a other than serine, amino acid 277 is other than glutamine, amino acid 462 is other than alanine, amino acid 456 is other than phenylalanine and amino acid 470 is other than aspartic acid. Particular alterations of the amino acid sequence set forth in SEQ. ID. NO. 30 include sequences in which amino acid 333 is other than threonine and/or amino acid 351 is a other than serine. One such altered IMPDH is a mutant mouse IMPDH type II [see, e.g., Lightfoot et al. (1994) Biochim. Biophys. Acta 1217:156-162 and SEQ ID NO. 32]. A nucleotide sequence encoding this altered mouse type II IMPDH is provided in SEQ ID NO. 31.

Inhibitor-resistant enzymes may be altered human IMPDH type II enzymes and include enzymes having substantially the same amino acid sequences set forth in SEQ. ID. NOS. 4, 6, 8, 10 and 12. DNA encoding an altered human IMPDH type II enzyme with an amino acid sequence set forth in SEQ. ID. NO. 4 (or as set forth in SEQ. ID. NO. 4 except that amino acids 190 and 191 are alanine and glycine, respectively) has been generated by site-directed mutagenesis. A nucleotide sequence encoding this altered human type II IMPDH is provided in SEQ. ID. NO. 3 (or the sequence of nucleotides 48 to 1589 of SEQ. ID. NO. 3 except that the sequence of nucleotides 614-619 is TGCAGG instead of CCGCAG).

DNA encoding enzymes having substantially the same amino acid sequences set forth in SEQ. ID. NOS. 6, 8, 10 and 12 have been generated using random mutagenesis of a wild-type IMPDH-encoding cDNA and selection of cells containing the mutagenized DNA based on resistance to MPA [see Farazi et al. (1997) J. Biol. Chem. 272:961-965]. Nucleotide sequences encoding these altered IMPDH enzymes are provided in SEQ. ID. NOS. 5, 7, 9 and 11. The altered enzymes had decreased affinity for MPA as follows: mutation of Q277R (see SEQ ID NO. 6) resulting in a 6-fold increase in the Kᵢ for MPA relative to wild-type human IMPDH type II enzyme, mutation of A462T (see SEQ ID NO. 8) resulting in a 3-fold increase in the Kᵢ for MPA relative to wild-type human IMPDH type II enzyme, double mutation of Q277R/A462T (see SEQ ID NO. 10) resulting in a 6-fold increase in the Kᵢ for MPA relative to wild-type human IMPDH type II enzyme with wild-type levels of activity, and mutation of F456S/D470G (see SEQ ID NO. 12) resulting in a 3-fold increase in the Kᵢ for MPA relative to wild-type human IMPDH type II enzyme.

4. Design of Selection Systems Based in a Resistant IMPDH Enzyme as a Selectable Marker

Selection systems using a resistant IMPDH enzyme as a selectable marker may be used with a variety of cells and in a variety of formats. Selection of a particular format may involve considerations of factors such as cell type, resistant enzyme that is being used as a selectable marker and properties of the selection agent.

a. Cells

Different cells, as well as the same cells under differing conditions, differentially utilize the two IMPDH isoforms and the de novo and salvage pathways for purine biosynthesis. For example, de novo purine synthesis is pivotal for the proliferative responses of T and B lymphocytes to mitogens [see, e.g., Sievers et al. (1997) Pharmacotherapy 17:1178-1197]. IMPDH activity and mRNA levels are induced up to 15-fold upon mitogenic or antigenic stimulation of human peripheral blood T lymphocytes [see Zimmermann et al. (1995) J. Biol. Chem. 270:6808-6814]. Lymphocytes are not able to synthesize guanine nucleotides via the salvage pathway [see Wu (1994) Perspect. Drug Discovery Des. 2:185-204]. Most cells, however, are able to use both the de novo and salvage pathways for purine nucleotide biosynthesis.

Cells, such as lymphocytes, that rely predominantly on the de novo pathway for guanine synthesis, rather than the salvage pathway, are most significantly affected by inhibitors of IMPDH. Generally, cells that can exhibit increased proliferation, such as proliferative responses to mitogens, antigens and other induction agents, tend to be most significantly affected by IMPDH inhibitors. For example, proliferation of mesangial cells located in the glomeruli of the kidneys is reduced by inhibitors of IMPDH [see Hauser et al. (1999) Nephrol. Dial. Transplant. 14:58-63]. In contrast, cells that are able to utilize the salvage pathway to synthesize purine nucleotides, and/or that are not subject to substantial increased proliferation, may not be significantly affected by IMPDH inhibitors.

IMPDH type I mRNA is the dominant isoform expressed in normal human lymphocytes [see Natsumeda and Carr (1993) Ann. N.Y. Acad. Sci. 696:88-93]. Proliferating lymphocytes, however, use IMPDH type II, the predominant enzyme isoform found in lymphocytes that are undergoing a proliferative response, in de novo guanine synthesis. Nonreplicating cells rely on the IMPDH type I isoform for de novo purine nucleotide synthesis.

Cells that have elevated IMPDH type II activity levels, or that rely primarily on IMPDH type II for purine biosynthesis, such as, for example, activated lymphocytes, certain transformed tumor cells [see, e.g., Jackson et al. (1975) Nature 256:331-333, Collart et al. (1992) Cancer Res. 52:5826-5828, and Huberman et al. (1994) Adv. Exp. Med. Biol. 370:741-746], myeloid leukemia L1210 cells, leukemic cell lines K562 and HL-60, leukemic cells from patients with different types of leukemia [see, e.g., Konno et al. (1991) J. Biol. Chem. 266:506-509] and hybridomas, are more significantly affected by agents that preferentially inhibit IMPDH type II [see, e.g., Hatse et al. (1999) Biochem. Pharmacol. 58:539-555].

Because cells can differ in their response to inhibitors of IMPDH, the type of cell, and the conditions in which the cell is maintained, can be factors in determining the selection marker and selection agent used in the selection systems provided herein. The differential response of cells to inhibitors of IMPDH can also advantageously be the basis for selective inhibition of the proliferation of certain cells but not all cells, as discussed further in connection with use of the selection systems in vivo.

b. IMPDH Used as a Selectable Marker

Resistant IMPDHs for use in these selection systems include IMPDH type I and type II enzymes that are resistant to an inhibitor of wild-type IMPDH. Generally, resistant IMPDH type I enzymes are used as selectable markers in systems for selecting cells that predominantly express IMPDH type I, and resistant IMPDH type II enzymes are used as selectable markers in systems for selecting cells that predominantly express IMPDH type II. It is also possible, however, to use resistant IMPDH type II enzymes as selectable markers in systems for selecting cells that predominantly express IMPDH type I and to use resistant IMPDH type I enzymes as selectable markers in systems for selecting cells that predominantly express IMPDH type II.

It is further possible to use resistant IMPDH type I and resistant IMPDH type II enzymes together as the selectable marker in systems for selecting cells that predominantly express one of the isoforms of IMPDH or, particularly in systems for selecting cells that express both isoforms of IMPDH at levels that are sufficient to provide for some cell proliferation. A single resistant isoform of IMPDH may also be used as a selectable marker in systems for selecting cells that express both isoforms of IMPDH. In this format, the system could include two selection agents, one that effectively inhibits the type I isoform and another that inhibits the type II isoform, or could include a single selection agent that effectively inhibits IMPDH types I and II.

c. IMPDH Inhibitors Used as Selection Agents

Inhibitors for use in these selection systems include inhibitors of IMPDH type I and type II enzymes. Inhibitors may effectively inhibit only one of the two IMPDH isoforms or may effectively inhibit both forms. An inhibitor may effectively inhibit one isoform of IMPDH at one concentration but inhibit the other isoform, or both isoforms, at increased concentrations. For example, MPA is a five-fold more potent inhibitor of the type II isoform than the type I isoform of human IMPDH. Thus, at lower concentrations, MPA is an effective inhibitor of IMPDH type II, without significantly inhibiting IMPDH type I, whereas at higher concentrations, MPA may be an effective inhibitor of IMPDH type I in addition to being an effective inhibitor of IMPDH type II. Two inhibitors with differing potencies for inhibiting the two isoforms of IMPDH may also be used as a selection agent in these selection systems. Regardless of the profile(s) of the IMPDH inhibitor(s) used, an important feature of an IMPDH inhibitor(s) used in these particular selection systems is that it provides for a level of inhibition of IMPDH in cells undergoing selection such that at least cells that do not contain an inhibitor-resistant IMPDH selectable marker but that are otherwise substantially identical to cells that do contain the selectable marker will exhibit reduced proliferation and/or viability in the presence of the inhibitor compared to in the absence of the inhibitor.

5. In vitro Use of Selection Systems Based in a Resistant IMPDH Enzyme as a Selectable Marker

a. Use of the Selection Systems in Selecting Genetically Modified CElls that Predominantly Express the Type II Isoform of IMPDH

Selection systems based in a resistant IMPDH as a selectable marker may be used in several formats in connection with the transfer of nucleic acids into cells that predominantly express IMPDH type II, such as, for example proliferating lymphocytes. In one format, the cells may be transfected with DNA encoding an IMPDH type II enzyme that is resistant to an inhibitor of IMPDH type II and then cultured in the presence of the inhibitor. Inhibitor concentrations to be used in such selection systems are concentrations that provide for selective greater proliferation of cells containing the DNA encoding an inhibitor-resistant IMPDH relative to at least substantially identical cells that do not contain DNA encoding an inhibitor-resistant IMPDH. Such concentrations of inhibitor may be determined empirically by those of skill in the art. The inhibitor may be used at concentrations that provide for proliferation of cells containing DNA encoding an inhibitor-resistant IMPDH to the exclusion of proliferation of cells that do not contain DNA encoding an inhibitor-resistant IMPDH.

Cells that predominantly express IMPDH type II under only certain conditions should be exposed to those conditions at some time prior to exposure to the inhibitor of IMPDH type II. If the cells are not exposed to conditions that result in increased expression of IMPDH type II, and the inhibitor used as a selection agent is not an effective inhibitor of IMPDH type I, then identification and/or isolation of cells into which heterologous has been transferred based on favored proliferation of those cells may be limited. Thus, for example, selection of transfected lymphocytes using a system employing a type II IMPDH-specific inhibitor and an inhibitor-resistant IMPDH type II as selection agent and selectable marker, respectively, typically involves stimulation of active cell cycling prior to exposure of cells to inhibitor. Proliferation of the lymphocytes is induced by lymphocyte activation through, for example, administration of antigens, monoclonal antibodies, cytokines, growth factors and/or mitogens, or any activation condition known in the art.

In another format, the cells may be transfected with DNA encoding an IMPDH type I that is resistant to an inhibitor of IMPDH type II and then cultured in the presence of the inhibitor. Because the activities of human type I and type II IMPDH may differ, this system may find particular use when it is desired to alter the level of purine nucleotide synthesis and/or level of proliferation of the transfected cells (e.g., if the cells are lymphocytes). Alternatively, if the IMPDH type I selectable marker is expressed at fairly high levels in the transfected cells (e.g., if the marker gene is present and expressed in multiple copies in transfected cells), then the level of IMPDH activity may provide for equivalent function, e.g., proliferation, as would be obtained in the absence of the inhibitor of IMPDH type II. If the inhibitor is one that also effectively inhibits IMPDH type I, then the resistant IMPDH type I should also be resistant to the type I inhibitory effects of the inhibitor. For example, MPA is a significantly more effective inhibitor of type II IMPDH than of type I IMPDH. However, if the limited inhibitory effect of MPA on human IMPDH type I is sufficient to inhibit proliferation of cells that predominantly express IMPDH type II that have been transfected with IMPDH type I-encoding DNA, then, to optimize selection of transfected cells, it may be preferable to utilize a type I IMPDH that is more resistant to MPA.

In a further format, the cells may be transfected with DNA encoding an IMPDH type II that is resistant to an inhibitor of IMPDH type II and an IMPDH type I that is resistant to an inhibitor of IMPDH type I and then cultured in the presence of an agent that effectively inhibits both isoforms of IMPDH or in the presence of two agents, each of which inhibits one of the two isoforms. This format may be of particular use with cells that express sufficient levels of IMPDH type I to permit proliferation of the cells in the presence of an inhibitor of IMPDH type II. In such a situation, it may also be possible to transfect the cells with DNA encoding an IMPDH type II that is resistant to an inhibitor of IMPDH type II and culture the cells in the presence of an agent or agents that inhibit both isoforms of IMPDH. In this format, the IMPDH type II would also have to be resistant to the inhibitor of IMPDH type I.

b. Use of the Selection Systems in Selecting Genetically Modified Cells that Predominantly Express the Type I Isoform of IMPDH

Selection systems based in a resistant IMPDH as a selectable marker may be used in several formats in connection with the transfer of nucleic acids into cells that predominantly express IMPDH type I. In one format, the cells may be transfected with DNA encoding an IMPDH type I enzyme that is resistant to an inhibitor of IMPDH type I and then cultured in the presence of the inhibitor.

In another format, the cells may be transfected with DNA encoding an IMPDH type II that is resistant to an inhibitor of IMPDH type I and then cultured in the presence of the inhibitor. If the inhibitor is one that also effectively inhibits wild-type IMPDH type II, then the IMPDH type II should also be resistant to the type II inhibitory effects of the inhibitor. Because the activities of human type I and type II IMPDH may differ, this system may find particular use when it is desired to alter the level of purine nucleotide synthesis and/or level of proliferation of the transfected cells. If the inhibitor is one that also effectively inhibits IMPDH type II, then the resistant IMPDH type II should also be resistant to the type II inhibitory effects of the inhibitor.

In a further format, the cells may be transfected with DNA encoding an IMPDH type II that is resistant to an inhibitor of IMPDH type and an IMPDH type I that is resistant to an inhibitor of IMPDH type I and then cultured in the presence an agent that effectively inhibits both isoforms of IMPDH or in the presence of two agents, each of which inhibits one of the two isoforms. This format may be of particular use with cells that express sufficient levels of IMPDH type II to permit proliferation of the cells in the presence of an inhibitor of IMPDH type I. In such a situation it may also be possible to transfect the cells with DNA encoding an IMPDH type I that is resistant to an inhibitor of IMPDH type I and culture the cells in the presence of an agent or agents that inhibit both isoforms of IMPDH. In this format, the IMPDH type I would also have to be resistant to the inhibitor of IMPDH type II.

c. Use of the Selection Systems in Selecting Genetically Modified Cells that are Able to Utilize the Salvage Pathway for Purine Biosynthesis

With certain cells, the use of selection systems that are based in a resistant IMPDH as a selectable marker and IMPDH inhibitors as selection agents may not provide sufficient inhibition of proliferation of non-transfected cells due to purine biosynthesis via the salvage pathway. Cells that have sufficient purine biosynthesis via the salvage pathway that are not sufficiently inhibited in their proliferation by the presence of IMPDH inhibitors can be used with these selection systems when an inhibitor(s) of salvage pathway enzymes is included in the selection agent.

Enzymes of the purine salvage pathway include, but are not limited to, hypoxanthine guanine phosphoribosyltransferase (HGPRT) and adenine phosphoribosyltransferase. Inhibitors of the purine synthesis salvage pathway include allopurinol, which exerts an inhibitory effect by increasing the intracellular concentration of hypoxanthine which leads to inhibition of HGPRT. Inhibitor-resistant or altered salvage pathway enzymes may also be used as selectable markers alone or in combination with inhibitor-resistant or altered enzymes from the de novo pathway along with inhibitors of the enzymes as selection agents.

B. Selection Systems Using Pyrimidine Biosynthesis Pathway Enzymes as a Selectable Marker

In another embodiment of the selection systems provided herein, an inhibitor-resistant or altered enzyme of a pyrimidine biosynthesis pathway is used as a selectable marker. Enzymes of the pyrimidine biosynthesis pathway include, but are not limited to, dihydroorotate dehydrogenase (DHODH), carbamoyl phosphate synthetase II, aspartate transcarbamylase, dihydroorotase, orotate phosphoribosyl transferase, OMP decarboxylase, CTP synthase, thymidylate synthase, and ribonucleotide reductase. Inhibitors of these enzymes, which may be useful as selection agents in systems using inhibitor-resistant forms of these enzymes as selectable markers, are known and can be identified by those of skill in the art. Included among such inhibitors are: acivicin, an inhibitor of carbamoyl phosphate synthetase II and CTP synthase, pyrazofurin, an inhibitor of OMP decarboxylase, hydroxyurea, an inhibitor of ribonucleotide reductase, and methotrexate and 5-fluorouricil, inhibitors of thymidylate synthase [Weber et al. (1991) Adv. Enzyme Regul. 31:45-67]. Enzymes resistant to such inhibitors are known or can be generated, for example, using methods provided herein or known to those of skill in the art.

The inhibitor-resistant or altered enzyme of a pyrimidine biosynthesis pathway used as a selectable marker may be a rate-limiting enzyme of de novo pyrimidine nucleotide synthesis. For example, in some selection systems, an inhibitor-resistant or altered dihydroorotate dehydrogenase (DHODH) enzyme (particularly a human DHODH), which serves as a selectable marker, is used to confer on cells in which it is expressed resistance to an inhibitor of DHODH (i.e., a selection agent). If the selectable marker is an altered DHODH, the alteration in the DHODH is such that the enzyme is no longer effectively inhibited by the selection agent.

1. Dihydroorotate Dehydrogenase (DHODH)

Dihydroorotate dehydrogenase (DHODH) is the rate-limiting enzyme of the de novo pyrimidine biosynthesis pathway, and is the molecular target of a number of antiproliferative and immunosuppressive compounds [see, e.g., Davis et al. (1997) Biochem. Pharmacol. 54:459-465; and Halloran (1996) Clin. Transplantation 10:118-123]. The de novo pathway begins with the synthesis of carbomoyl phosphate from HCO₃⁻ and the amide nitrogen of glutamine by the cytosolic enzyme carbamoyl phosphate synthetase II. Carbamoyl phosphate is then condensed with aspartate to form carbamoyl aspartate catalyzed by aspartate transcarbamylase. Dihydroorotase then catalyzes an intramolecular condensation forming dihydroorotate. Dihydroorotate is irreversibly oxidized to orotate by DHODH. Orotate reacts with PRPP to yield orotidine-5′-mono-phosphate (OMP) catalyzed by orotate phosphoribosyl transferase. Finally, OMP undergoes a decarboxylation step catalyzed by OMP decarboxylase to form UMP.

The oxidation of dihydroorotate to orotate by DHODH is achieved by transferring electrons to ubiquinone (QO) through an enzyme-bound redox cofactor (flavin). In one proposed mechanism, the enzyme follows a two site ping-pong mechanism, with dihydroorotate oxidized at one site and Q₀ reduced at the other with different faces of the flavin interacting with each substrate (Neidhardt, et al. (1999) J. Mol. Microbiol. Biotechnol. 1(1):183-188). Prokaryotic and fungal forms of the enzyme have been shown to be either soluble or associated with the inner membrane. Mammalian DHODH is associated with the mitochondrial inner membrane (Neidhardt, et al., (1999) J. Mol. Microbiol. BiotechnoL. 1(1):183-188).

A salvage pathway for pyrimidine biosynthesis, which works by recycling pyrimidine bases and nucleosides, is also available in normal mammalian cells, in addition to the de novo pathway. The salvage pathway involves nucleoside phosphorylases and nucleoside kinases. For example, uracil can be salvaged to form UMP through the concerted action of uridine phosphorylase and uridine kinase. Deoxyuridine is also a substrate for uridine phosphorylase. The salvage pathway to dTMP synthesis involves the enzymes thymine phosphorylase and thymidine kinase. The salvage of deoxycytidine is catalyzed by deoxycytidine kinase.

Proliferating lymphocytes depend primarily on the de novo pathway for pyrimidine biosynthesis while most other cell types, including, for example, fibroblasts, smooth muscle cells, endothelial cells and intestinal epithelial cells, are able to use both the de novo and salvage pathways. In some cancer cells the activity of enzymes of the salvage pathway may be higher than those of the rate-limiting enzymes of de novo biosynthesis. Also, the metabolic flux through the salvage pathway in these cells may increase after treatment with inhibitors of de novo pyrimidine biosynthesis (Weber et al. (1991) Adv. Enzym. Regul 31:45-67).

DHODH activity, as well as the activity of other enzymes in the de novo and salvage pyrimidine biosynthesis pathways, is closely positively correlated with cell proliferation. Elevated DHODH activity has been observed with increased proliferation of both normal and malignant tissues and cells and also in tissues with known regenerative, absorptive or excretory activities [see, e.g., Loeffler et al. (1996) Histochemistry and Cell Biology 105(2):119-128 and Fairbanks et al. (1995) J. Biol. Chem. 270(50):29682-29689]. Inhibitors of DHODH activity have been shown to inhibit cell proliferation and induce cellular differentiation [see, e.g., Fairbanks et al. (1995) J. Biol. Chem. 270(50):29682-29689; Lyons et al. (1990) Biochem. International 22(6):939-950; Loeffler et al. (1992) Biochem. Pharmacol. 43(10):2281-2287; and Czech et al. (1999) Int. J. Immunotherapy 1(4):185-191].

2. Inhibitors of DHODH

Inhibitors of DHODH that may be used as selection agents in systems employing an inhibitor-resistant or altered DHODH as a selectable marker typically provide for a level of inhibition of DHODH in cells such that at least certain cells exposed to the inhibitor under certain conditions exhibit reduced proliferation relative to the amount of proliferation of the cells in the absence of the inhibitor. At minimum, cells that may exhibit reduced proliferation when exposed to the DHODH inhibitor are those that do not contain an inhibitor-resistant or altered DHODH that confers resistance to conditions that include the presence of the inhibitor, but that are otherwise substantially identical to cells that contain the resistant DHODH. Inhibitors of DHODH that may be used as selection agents in selection systems provided herein generally are ineffective at substantially inhibiting an inhibitor-resistant or altered DHODH that may be used as a selectable marker.

The ability of DHODH inhibitors to inhibit cell proliferation may be determined by a variety of methods. For example, generally, cell proliferation may be determined in the absence of and in the presence of increasing concentrations of inhibitor by measuring [³H]thymidine incorporation by cells as an assessment of DNA synthesis using standard methods known to those of skill in the art. Inhibition of cell proliferation by DHODH inhibitors may also be assessed in in vitro and in vivo cell proliferation assays described herein (see, for example, Examples 6 and 7) and/or known to those of skill in the art. For example, the procedure of Greaves et al. [(1974) Nature 248:698-701], and modified versions thereof (see, e.g., U.S. Pat. No. 5,380,879 and Fairbanks et al., (1995) J. Biol. Chem. 270(50):29682-29689), may be used to evaluate the in vitro effect of a compound on the responses of human peripheral blood lymphocytes to T- and B-cell mitogens. The ability of a compound to inhibit the proliferation of mouse mammary carcinoma cells (EMT6 cells) is a further assay that may be used to evaluate the effects of DHODH inhibitors on cell proliferation [see, e.g., Franklin et al. (1999) Biochem. Pharmacol. 58:867-876]. The hemolytic plaque forming cell assay procedure of Jerne et al. [Cellbound Antibodies, Amos and Kaprowski, eds. (Wistar Institute Press, Philadelphia, 1963) p. 109], and modified versions thereof (see, e.g., U.S. Pat. No. 5,380,879), may be used, for example, to evaluate the in vivo immunosuppressive activity of a compound, which is an indication of the effect of the compound on the ability of immune system cells to proliferate and generate a response to antigen. Another assay for the immunosuppressive activity of a compound utilizes a model of DTH in which the effect of the compound on the response of mice that had been immunized with ovalbumin to a challenge by intraplantar injection of ovalbumin is assessed by measuring footpad thickness as an indicator of the degree of inflammation [see, e.g., Franklin et al. (1999) Biochem. Pharmacol. 58:867-876].

DHODH inhibitors include, but are not limited to, cinchoninic acid derivatives such as brequinar (6-fluoro-2-(2′-fluoro-1,1′-biphenyl-4-yl)-3-methyl-4-quinoline carboxylic acid), naphthoquinone derivatives such as dichloroally lawsone, isoxazole derivatives such as leflunomide (N-(4-trifluoromethylphenyl)-5-methylisoxazol-4-carboxamide) and other immunosuppressive and antiproliferative compounds such as NSC 665564, redoxal and BNID.

a. Cinchoninic Acid or Quinolone Carboxylic Acid Derivatives

Brequinar, a synthetic difluoroquinolone carboxylic acid derivative, (NSC 368390) is a non-competitive inhibitor of the enzyme DHODH with antiproliferative and immunosuppressive properties [see, e.g., Xu et al. (1998) J. Immunol. 160:846-853]. Brequinar is reported to be a slow-binding inhibitor of human DHODH with a Kᵢ of 1.8 nM [see, e.g., Knecht and Loffler, (1998) Biochem. Pharmacol. 56:1259-1264]. Quinolone carboxylic acid derivatives, analogs and metabolites are also useful as selection agents in the systems provided herein. Numerous derivatives, analogs, metabolites, and processes for preparing such compounds have been reported, including but not limited to, those described in U.S. Pat. Nos. 5,084,462; 5,032,597; 4,968,701; 4,861,783; and 4,680,299.

b. Naphthoquinone Derivatives

The natural naphthoquinone compound lawsone and derivatives dichloroallyl lawsone (DCL), lapachol, and atovaquone are inhibitors of DHODH. Atovaquone (Kᵢ=2.71M) and DCL (Kᵢ=9.8 nM) have been shown to be competitive inhibitors of human DHODH with respect to ubiquinone. Atovaquone and DCL show uncompetitive behavior with respect to the substrate dihydroorotate (Atovaquone, Kᵢ=11.5 μM; DCL, Kᵢ=38 nm) [see, e.g., Knecht et al., (2000) Chemico-Biological Interactions 124:61-76]. Other naphthoquinone derivatives, analogs and metabolites are also useful as selection agents in the systems provided herein. Numerous derivatives, analogs, metabolites and methods of preparing such compounds have been reported, including, but not limited to, those described in U.S. Pat. Nos. 4,430,891; 3,655,599; and 4,530,845.

c. Isoxazole Derivatives

The isoxazole derivative Leflunomide is rapidly converted to its active open-ring form upon administration, (N-(4-trifluoromethylphenyl)-2-cyano-3-hydroxycrotonamide) (A771726). A771726 is an uncompetitive inhibitor with respect to the substrate dihydroorotate and a non-competitive inhibitor with respect to ubiquinone. Leflunomide has Kᵢ values in the range of 600-4,000 nM [See, e.g., Knecht and Loffler, (1998) Biochem. Pharmacol. 56:1259-1264 and Greene et al. (1995) Biochem. Pharmacol. 50:861-867]. Derivatives, analogs and metabolites of Leflunomide are also useful as selection agents in the systems provided herein. Numerous isoxazole derivatives, analogs, metabolites, and process for preparing such compounds have been reported, including, but not limited to, those described in Knecht and Loffler [(1998) Biochem. Pharmacol. 56:1259-1264] and U.S. Pat. Nos. 5,905,090; 4,087,535; 4,351,841; and 4,965,276.

d. Other

NSC 665564 is an inhibitor of the enzyme DHODH and is thought to have the same mechanism of inhibition as Brequinar. NSC 665564 has a comparable IC₅₀ to Brequinar and a lower Kᵢ [Cleaveland et al. (1996) Biochem. Biophys. Res. Commun. 223(3):654-659). Redoxal (2,2′-(3,3′-dimethoxy(1,1′-biphenyl)-4,4′-diyl)diimino)bis-benzoic acid) is a non-competitive inhibitor for both substrates of human DHODH, dihydroorotate and ubiquinone [Knecht and Loffler (1999) FEBS Letters 467:27-30]. Redoxal has an apparent Kᵢ of 330 nM. BNID (1-(p-bromophenyl)-2-methyl 1H-napth(2,3-d)-imidazole-4,9-dione) is also an inhibitor of DHODH with a Kᵢ of 530 nM [Cleaveland et al. (1995) Biochem. Pharmacol. 49(7):947-954]. Phenoxyquinolines, which have been reported to be plant fungicides, may also inhibit DHODH [see, e.g., U.S. Pat. No. 5,976,848]. Included in this class of compounds is 8-chloro-4-(2-chloro-4-fluorophenoxy)quinoline (CCFQ).

e. Methods of Identifying Inhibitors of DHODH

Inhibitors of DHODH may also be identified in a variety of screening assays. For example, candidate inhibitory compounds may be tested for ability to inhibit DHODH activity assessed in spectrophotometric assay. Two such assays are coupled assays, based on the ability of active enzyme to transfer electrons through ubiquinone to an acceptor (NBT or DCIP) in the presence of dihydroorotate. Thus, DHODH activity may be determined by monitoring the reduction of NBT or DCIP as a change in absorbance at 610 nm of a reaction mixture containing ubiquinone, dihydroorotate, DHODH and either NBT or DCIP [see, e.g., Dacis et al. (1997) Biochem. Pharmacol. 54(4):459-465].

In another method for determining inhibitors of DHODH, MOLT-4 cells are incubated with the drug for 18 hr followed by a 1-hr pulse with (14C) bicarbonate. Cellular accumulation of (14C) N-carbamyl-L-aspartic acid and (14C)L-dihydroorotic acid concurrent with marked depletion of CTP and UTP indicates inhibition of the enzyme DHODH [See, e.g., Cleaveland et al., (1995) Biochem. Pharmacol. 49(7):947-954, and Cleaveland et al., (1996) Biochem. Biophys. Res. Commun 223(3):654-650].

In another method for identifying inhibitors of DHODH, organisms auxotrophic for pyrimidines (due to lack of the endogenous gene for the bacterial DHODH or presence of a defective DHODH gene which fails to produce functional DHODH enzyme), for example, Escherichia coli strain TAP330 which lacks the endogenous gene for bacterial DHODH [see Davis et al. (1997) Biochem. Pharmacol. 54(4):459-565 and Copeland et al. (1995) Arch Biochem. Biophys. 323:79-86], that have been transformed with DNA encoding a DHODH enzyme of interest are exposed to candidate inhibitors. DHODH inhibitors are identified by an ability to inhibit transformed cell proliferation. Specificity of the inhibitor for DHODH is evaluated by supplementing the transformed cell culture media with pyrimidine nucleotide precursors to correct the defect.

3. Inhibitor-Resistant DHODH Enzymes

Inhibitor-resistant DHODH enzymes useful as selectable markers in particular selection systems described herein provide in cells in which they are expressed increased resistance to inhibition of cell proliferation and/or reduction of cell viability by an inhibitor of a DHODH relative to the resistance of cells that do not express the inhibitor-resistant DHODH. Resistance to inhibition of cell proliferation and/or reduction of cell viability can be assessed using methods described herein or known in the art. For example, cells can be grown in media containing incrementally increasing concentrations of a DHODH inhibitor, and cell number at each concentration may be determined using standard techniques such as a Coulter counter [see, e.g., Hodges et al. (1989) J. Biol. Chem. 264:18137-18141]. Viable cell numbers may also be determined at each concentration using methods described herein and known to those of skill in the art.

Cell proliferation may be determined, for example, by measuring [³H]thymidine incorporation by cells as an assessment of DNA synthesis using standard methods known to those of skill in the art. The results can be evaluated in a number of ways. For example, the concentration of inhibitor that results in a 50% reduction of cell proliferation over a certain time period, i.e., the IC₅₀ value, can be determined. Results may also be evaluated as a plot of the reciprocal fraction of the mean doubling time of inhibitor-treated cultures over a particular growth period compared to the doubling time in the absence of inhibitor versus inhibitor concentration.

Cells expressing an inhibitor-resistant or altered DHODH enzyme will exhibit greater proliferation and/or viability at increasing concentrations of inhibitor than cells that express a sensitive or unaltered DHODH enzyme. Therefore, a higher concentration of inhibitor is required to achieve the same level of reduction in cell proliferation and/or cell viability in cells expressing an inhibitor-resistant DHODH as compared to cells expressing a sensitive DHODH. Thus, cells expressing an inhibitor-resistant DHODH enzyme will exhibit increased resistance to inhibition of cell proliferation and/or reduction of cell viability by a DHODH inhibitor. Some inhibitor-resistant DHODH enzymes for use as selectable markers may provide in cells in which they are expressed about 2- to 10,000-fold or greater (typically about 5- to 2500-fold or 10- to 500-fold) increased resistance to inhibition of cell proliferation by an inhibitor of an DHODH. This can be expressed, for example, as a 2- to 10,000-fold increase in the IC₅₀ value for growth inhibition for the inhibitor of DHODH.

Increased resistance to inhibition of cellular proliferation can be the result of increased Kᵢ of an inhibitor-resistant DHODH (as compared to a inhibitor-sensitive or unaltered or wild-type DHODH) or the combination of increased Kᵢ and increased expression (i.e., resulting in increased protein levels) of an inhibitor-resistant DHODH in resistant cells relative to the amount of expression of an inhibitor-sensitive DHODH in non-resistant cells. It is possible for increased resistance to inhibition of cell proliferation to be achieved by virtue of the reduced sensitivity of the inhibitor-resistant DHODH for an inhibitor such that substantially increased expression is not required to effect selection of cells containing the inhibitor-resistant DHODH. Thus, selection of genetically modified cells is achieved in these selection systems through introduction and expression of as few as about 1 to about 10, or about 2 to about 10 or about 1 to about 5 or about 2 to about 5 or about 2 copies or 1 copy of a nucleic acid encoding an inhibitor-resistant DHODH into a cell. Thus, increased resistance to inhibition of cell proliferation and/or reduction of cell viability may be achieved by virtue of the reduced sensitivity of the inhibitor-resistant DHODH to an inhibitor without depending on increased levels of expression of the inhibitor-resistant DHODH.

Indications of increased expression or gene amplification include increased mRNA and/or protein levels, increased enzyme activity when cells are cultured in the presence of inhibitor and decreased enzyme activity when cells are cultured in the absence of inhibitor. Changes in the kinetic or inhibition parameters (e.g., the Kₘ and Kᵢ values) of an inhibitor-resistant or altered DHODH relative to those of an inhibitor-sensitive or unaltered DHODH are reflective of an inherent reduced sensitivity of the enzyme irrespective of the amount of enzyme that may be present.

Generally, such DHODH enzymes which provide for increased resistance to inhibition of cell proliferation and/or reduction of cell viability by a DHODH inhibitor are not effectively inhibited by one or more conditions that can inhibit a DHODH enzyme. Such enzymes may be from an organism that expresses a DHODH that has an inhibitor sensitivity that differs from that of DHODH enzymes from another organism. For example, the rat and human DHODH enzymes differ in sensitivities to brequinar and isoxazol derivatives [see, e.g., Knecht and Loffler (1998) Biochem. Pharmacol. 56:1259-1264]. From the IC₅₀ values, it has been deduced that brequinar is a more potent inhibitor of the human DHODH activity (IC₅₀=10 nM) than of the rat enzyme (IC₅₀ 367 nM). The rat DHODH enzyme is influenced by isoxazol derivatives to a greater extent (IC₅₀=19 nM A77-1726, i.e., the active metabolite of leflunomide) than the human enzyme (IC₅₀=1.1 μM A77-1726).

DHODH enzymes that provide for increased resistance to inhibition of cell proliferation and/or reduction of cell viability by a DHODH inhibitor may also be a DHODH that is altered relative to wild-type DHODH such that an inhibitor of wild-type DHODH is no longer an effective inhibitor of the enzyme. Although the activity of the altered DHODH enzyme may differ from that of the wild-type enzyme, it must be sufficient to effectively catalyze the oxidation of dihydroorotate to orotate and thereby maintain cellular pyrimidine pools required for DNA and RNA biosynthesis.

In particular embodiments of the selection systems using inhibitor-resistant, including altered, DHODH as a selectable marker, the resistant DHODH enzymes are nonimmunogenic or minimally immunogenic with respect to host organisms in which they may be used. Immunogenicity of altered DHODH enzymes may be evaluated using a variety of assays such as those described herein and known in the art.

a. Catalytic Activity and Inhibitor Sensitivity of Inhibitor-Resistant DHODH Enzymes

Inhibitor-resistant DHODH enzymes are insensitive to or have reduced sensitivity to one or more inhibitors of DHODH enzymes. The catalytic activity of altered DHODH enzymes is substantially similar to or at least not significantly reduced relative to the catalytic activity of wild-type DHODH enzymes.

The activity and inhibitor sensitivity of inhibitor-resistant or altered DHODH enzymes may be evaluated using assays as described herein or any assay known in the art for measuring DHODH activity. Two such methods involve calorimetric assays, the nitroblue tetrazolium (NBT) assay and 2,6-dichloroindophenol (DCIP) assay [see, e.g., Davis et al. (1997) Biochem. Pharmacol. 54(4):459-465, and Knecht et al. (1999) Chemico-Biological Interactions 124:61-76]. Both assays are coupled assays, based on the ability of active enzyme to transfer electrons through ubiquinone to an acceptor (NBT or DCIP) in the presence of dihydroorotate. The reduction of these compounds produces a change in their absorption spectra which can be monitored spectrophotometrically.

In the case of the NBT assay, reduction of NBT is accompanied by an increase in absorbance at 610 nm which is monitored as a function of time. Generally, the reaction mixture (200 μL total volume) contains 200 μM ubiquinone in an assay buffer (100 mM Tris, pH 8.0, 0.1% Triton X-100), 250 μM dihydroorotate, and 100 μM NBT. The reaction is initiated by the addition of dihydroorotate and NBT. Alternatively, the reaction can be initiated by addition of enzyme. For inhibitor studies, 10 μL inhibitor is incubated for 5 min with the enzyme/ubiquinone solution prior to addition of dihydroorotate and NBT. This assay is linear over a wider enzyme concentration range compared to the DCIP assay and is therefore convenient for conditions employing either long incubation times (to test for residual activity) or high enzyme concentrations (for inhibitor titration studies). An estimate of the relative activity of the mutant compared to wild-type can be made by dividing the Vₘₐₓ values for the two enzymes (determined from the NBT assay) by the respective total amount of protein. Total protein can be quantitated using the Bradford assay.

In the case of the DCIP assay, reduction of DCIP is stoichiometrically equivalent to oxidation of dihydroorotate and is accompanied by a loss of absorbance at 610 nm which is monitored as a function of time. In general, the reaction mixture (200 μL total volume) contains 200 μM ubiquinone in assay buffer (100 mM Tris, pH 8.0, 0.1% Triton X-100), 250 μM dihydroorotate, and 100 μM DCIP. The reaction is initiated by the addition of dihydroorotate and DCIP. Alternatively, the reaction can be initiated by addition of enzyme. For determining the Km relative to dihydroorotate and ubiquinone, the reaction mixture contains a constant ubiquinone concentration (200 μM) and varying concentrations of dihydroorotate (0-100 μM) or a constant concentration of 250 μM dihydroorotate and varying concentrations of ubiquinone (0-100 μM) respectively. The data from each experiment can then be fit to the Henri-Michaelis-Menten equation using a nonlinear least squares method to determine values of Kₘ and Vₘₐₓ.

To determine catalytic turnover constants, k_{cat} values, an active site titration procedure can be used. In this procedure, an inhibitor of DHODH can be used to titrate the amount of active DHODH present in cell lysate, isolated mitochondria or solution after purification. Prior to substrate addition, the enzyme is incubated with 0-100 nM inhibitor for 5 min. The fractional velocity as a function of inhibitor concentration is determined, and the total enzyme concentration can be calculated by direct fit of the data to the Morrison equation.

The data obtained from DHODH activity assays in the presence and absence of inhibitor may be used to determine the Kₘ and k_{cat} values of the inhibitor-resistant or altered DHODH enzyme and the Kᵢ for the inhibitor. The Kₘ value for human DHODH is about 9.4 μM for dihydroorotate, about 13.7 μM for decylubiqunone (QD) [see, e.g., Knecht et al. (2000) Chemico-Biological Interactions 124:61-76] and about 110 μM for ubiquinone (coenzyme Q) [see, e.g., Neidhardt et al. (1999) J. Mol. Microbiol. Biotechnol. 1:183-188]. Orotate is an uncompetitive inhibitor with respect to quinone [Kᵢⱼ(intercept inhibition constant)=60 ±10 μM] and a competitive inhibitor with respect to dihydroorotate [Kᵢₛ(slope inhibition constant)=22±3 μM]. Some inhibitor-resistant or altered DHODH enzymes may have a Kᵢ for inhibitor that is in the range of about 1.5-fold to about 2500 (or more)-fold or about 3-fold to 2500-fold or about 3-fold to about 500-fold greater than the Kᵢ of the inhibitor-sensitive or wild-type enzyme for the inhibitor. Some inhibitor-resistant or altered DHODH enzymes may be less sensitive to inhibitor than inhibitor-sensitive or wild-type enzyme by a factor of about 1.5-fold to 2500 (or more)-fold or about 3-fold to 2500-fold or about 3-fold to 500-fold or about 5-fold to 200-fold. Sensitivity can be expressed, for example, as the IC₅₀ (i.e., the inhibitor concentration that effects half-maximal inhibition, which will be greater for less sensitive enzymes) value of an enzyme for an inhibitor, as can be determined, for example, by plotting the fractional velocity of the enzyme (vᵢ/vₒ) versus inhibitor concentration [see, e.g., Davis et al (1997) Biochem. Pharmacol. 54(4):459-465]. Some altered DHODH enzymes may have a Kₘ for dihydroorotate and/or ubiquinone that is at least similar to that of the inhibitor-sensitive or wild-type enzyme or within a range of about 1.5-fold to about 5-fold or about 1.5-fold to about 3-fold of the of the inhibitor-sensitive or wild-type enzyme. Some inhibitor-resistant or altered DHODH enzymes may have a k_{cat} value that is at least similar to that of the inhibitor-sensitive or wild-type enzyme or within a range of 0.1- to 0.9-fold that of the inhibitor-sensitive or wild-type enzyme.

b. Generation of Resistant DHODH Enzymes

Altered DHODH enzymes may be obtained by a variety of means. For example, altered DHODH-encoding DNA may be generated by random mutagenesis of the cDNA encoding a wild-type DHODH enzyme followed by selection of cells containing the mutagenized cDNA for the ability to grow in the presence of an inhibitor of wild-type DHODH. Site-directed mutagenesis of a wild-type DHODH-encoding DNA may also be used to obtain a resistant DHODH-encoding DNA.

(1) Random Mutagenesis

One method of generating altered DHODH-encoding DNA involves transferring a complementary DNA encoding a wild-type DHODH enzyme (such as that having a coding sequence substantially similar to that set forth as nucleotides 1 to 1188 in SEQ ID NO. 19 or nucleotides 4 to 1101 in SEQ ID NO. 21) into a mutator strain of Escherichia col, such as mutD5 E. coli strain NR9072 [see, e.g., Schaaper (1988) Proc. Natl. Acad. Sci. U.S.A. 85:8126-8130]. Spontaneous mutations of the wild-type DHODH cDNA will occur when it is contained on a plasmid that is introduced into mutator strains which have defects in processes such as exonucleolytic proofreading during DNA replication and mutHLS-encoded mismatch correction.

Plasmid containing the DHODH-encoding cDNA is then isolated from a culture of the transformed mutD5 E. coli and used to transform an E. coli strain that is defective in DHODH activity, e.g., E. coli strain TAP330 which lacks the endogenous gene for bacterial DHODH [see Davis et al., (1997) Biochem. Pharmacol. 54(4):459-565 and Copeland et al., (1995) Arch Biochem. Biophys. 323:79-86]. A wild-type human DHODH-encoding cDNA can complement the deficiency of TAP330 cells. Inhibitor-resistant clones of the TAP330 cells transformed with the library of randomly mutagenized DHODH-encoding cDNA may be selected by growth on minimal medium containing the inhibitor.

The inhibitor-resistant clones are then characterized to determine if there is an alteration in the DHODH-encoding cDNA that reduces or eliminates its sensitivity to the inhibitor yet does not significantly reduce the catalytic activity of the enzyme. For example, DHODH enzyme can be purified from cell lysate using purification techniques known in the art and then assayed for activity using assays as described herein or any assay known in the art for measuring DHODH activity.

(2) Site-Directed Mutagenesis

Altered DHODH-encoding DNA may also be generated by site-directed mutagenesis. One method for performing site-directed mutagenesis employs a PCR mutagenesis technique [see, e.g., Innis et al. (1990) PCR Protocols: A Guide to Methods and Applications]. This technique requires two rounds of PCR amplification and four oligonucleotide primers, two inside primers and two outside primers. The two inside primers are of opposite orientation and contain a region of complementarity (greater than or equal to 13 bases), with at least one primer incorporating the desired mutation. The outside primers are complementary to the template and define the 5′ and 3′ limits of the fragment to be amplified. In the first round of PCR, the DHODH plasmid is used as a template for two separate reactions in which (1) a forward outside primer is combined with a reverse inside primer, and (2) a reverse outside primer is combined with a forward inside primer. Following purification of the two PCR products, a second round of PCR is initiated combining the first two PCR products. The fragments from the first round of PCR contain a region of complementarity as defined by the original inside primer pair and, therefore, serve as the template. The outside primers are used to amplify the full-length fragment.

To reduce the risk of base misincorporation, additional unique restriction sites can be incorporated into the DHODH template at intervals of approximately 300 bp to minimize the size of the amplified DNA fragment. Restriction sites can be introduced using general PCR mutagenesis techniques described above. The DHODH plasmid incorporating additional restriction sites is then used as the template for the two separate reactions in the first round of PCR as described above. In the first reaction, the forward primer containing the mutation is combined with a reverse primer containing the nearest unique restriction site. In the second reaction, the reverse primer containing the mutation is combined with a forward primer containing the nearest restriction site. Following purification, the two PCR products are combined in a second round of PCR and amplified with the outside primers containing the nearest restriction sites. This fragment is then digested by the nearest unique restriction enzymes and subcloned into a DHODH plasmid from which the corresponding wild-type fragment has been removed. Incorporation of the desired mutation and absence of nonspecific mutations can be confirmed by fluorescent cycle sequencing of each plasmid.

DHODH mutant plasmid can then be transformed into a pyrimidine auzotrophic strain of E. colI, TAP330, which lacks the endogenous gene for the bacterial DHODH. The DHODH mutants are then characterized to determine if the mutation reduces or eliminates its sensitivity to the inhibitor yet does not significantly reduce the catalytic activity of the enzyme. For example, cell lysate can be use directly or DHODH enzyme can be purified using metal-chelate chromatography and cation exchange chromatography and then assayed for activity using assays as described herein or any assay known in the art for measuring DHODH activity.

C. Exemplary Inhibitor-Resistant DHODH Enzymes

Inhibitor-resistant DHODH enzymes include wild-type enzymes with reduced sensitivity to inhibitors of other wild-type enzymes and altered DHODH enzymes, e.g., mutant Aspergillus nidulans DHODH [see U.S. Pat. No. 5,976,848, Nov. 2, 1999] resistant to the phenoxyquinoline CCFQ (8-chloro-4-(2-chloro-4-fluoro-phenoxy)quinoline) and altered human DHODH with reduced sensitivity to brequinar [see, e.g., the H26A mutant human DHODH reported by Davis et al. (1997) Biochem. Pharmacol. 54(4):459-465]. Thus, altered DHODH enzymes include enzymes having substantially the same amino acid sequences set forth in SEQ. ID. NOS. 20, 22, and 16 but that are altered such that the alteration(s) imparts on the enzyme resistance to a DHODH inhibitor. Such altered enzymes include those containing substantially the same amino acid sequence as set forth in SEQ. ID. NOS. 26 and 28. Exemplary DNA encoding such sequences includes the sequence set forth in SEQ. ID. NOS. 25 and 27, which are sequences encoding mutant A. nidulans DHODH enzymes resistant to CCFQ. Other altered DHODH enzymes include enzymes containing substantially the same amino acid sequence as set forth in SEQ. ID. NO. 20 or SEQ. ID. NO. 22 except that the amino acid at position 134 or 105, respectively, is other than valine, e.g., glutamic acid.

Inhibitor-resistant enzymes may be altered human DHODH enzymes containing substantially the same amino acid sequence as set forth in SEQ. ID. NO. 20 or SEQ. ID. NO. 22, except that amino acid residue 56 or 27, respectively, is other than histidine. Included among such enzymes is one containing substantially the same amino acid sequence as set forth in SEQ ID NO. 24. DNA encoding an altered human DHODH enzyme having substantially the same amino acid sequence set forth in SEQ ID NO. 24 has been generated using PCR site-directed mutagenesis [see, e.g., Davis et al. (1997) Biochem. Pharmacol. 54(4):459-465]. The H26A mutant of DHODH has a decrease in sensitivity to the inhibitor Brequinar of at least 167-fold relative to the wild-type human enzyme with comparable Kₘ values for dihydroorotate and ubiquinone and a 10-fold decrease in Vₘₐₓ [see Davis et al. (1997) Biochem. Pharmacol. 54(4):459-465].

4. Design and in vitro Use of Selection Systems Based in a Resistant DHODH Enzyme as a Selectable Marker

Selection systems using a resistant DHODH enzyme as a selectable marker may be used with a variety of cells and in a variety of formats. Selection of a particular format may involve considerations of factors such as cell type, resistant enzyme that is being used as a selectable marker and properties of the selection agent.

a. Cells

Different cells, as well as the same cells under differing conditions, differentially utilize the de novo and salvage pathways for pyrimidine biosynthesis. For example, de novo pyrimidine synthesis is pivotal for the proliferative responses of lymphocytes to mitogens [see, e.g., Fairbanks et al., (1995) J. Biol. Chem. 270(50):29682-29689]. Only a small amount of UTP synthesis is needed in resting lymphocytes which are, therefore, able to meet their metabolic requirements predominantly by pyrimidine salvage pathways. Proliferating lymphocytes require a higher rate of UTP production for DNA synthesis and for the massive expansion in phospholipid biosynthesis. Pyrimidine pools have been shown to increase up to 8-fold upon mitogenic or antigenic stimulation of human blood T lymphocytes [Fairbanks et al. (1995) J. Biol Chem. 270(50):29682-29689]. The metabolic requirement of proliferating lymphocytes is fulfilled predominantly by de novo pyrimidine biosynthesis.

Generally, cells that can exhibit increased proliferation, such as proliferative responses to mitogens, antigens and other induction agents, or exhibit known regenerative, absorptive or excretory activities (e.g., mucosal cells of the ileum, colon crypts in the gastrointestinal tract, cultured Ehrlich ascites tumor cells, and proximal tubule of the kidney cortex) have higher DHODH activity levels [see, e.g., Loeffler, et al., (1996) Histochem. and Cell Biol. 105(2):119-128]. Such cells rely predominantly on the de novo pathway for uridine synthesis, rather than the salvage pathway, and are therefore most significantly affected by inhibitors of DHODH. For example, proliferation of leukemia cells and formation of neointimal hyperplasia, characterized by proliferation of vascular smooth muscle cells in vessel walls, is reduced by inhibitors of DHODH [see e.g., Lyons et al., (1990) Biochem. International 22(6):939-950 and Czech et al., (1999) Int. J. Immunotherapy 1(4):185-191]. In contrast, cells that are able to utilize the salvage pathway to synthesize pyrimidines, have lower DHODH activity, and/or that are not subject to substantial increased proliferation, may not be significantly affected by DHODH inhibitors (e.g., periportal area of the liver, testis and spermatozoa, prostate and other glands, skeletal muscle, and some cancer cells) [see, e.g., Loeffler, et. al., (1996) Histochem. and Cell Biol. 105(2):119-128 and Weber et al., (1991) Adv. Enzyme. Regul. 31:45-67 ].

Because cells can differ in their response to inhibitors of DHODH, the type of cell, and the conditions in which the cell is maintained, can be factors in determining the selection marker and selection agent used in the selection systems provided herein. The differential response of cells to inhibitors of DHODH can also advantageously be the basis for selective inhibition of the proliferation of certain cells but not all cells, as discussed further in connection with use of the selection systems in vivo.

b. Use of the Selection Systems in Selecting Genetically Modified Cells that Rely Predominantly on the de novo Pathway for Pyrimidine Synthesis and/or Express Elevated DHODH Activity

Selection systems based on a resistant DHODH as a selectable marker may be used in several formats in connection with the transfer of nucleic acids into cells that rely predominantly on the de novo pathway for pyrimidine nucleotide synthesis, such as, for example proliferating lymphocytes. In one format, the cells may be transfected with DNA encoding a DHODH enzyme that is resistant to an inhibitor of DHODH and then cultured in the presence of the inhibitor. An important feature of a DHODH inhibitor used in these particular selection systems is that it provides for a level of inhibition of DHODH in cells undergoing selection such that at least cells that do not contain an inhibitor-resistant DHODH selectable marker but that are otherwise substantially identical to cells that do contain the selectable marker will exhibit reduced proliferation in the presence of the inhibitor compared to in the absence of the inhibitor.

Inhibitor concentrations to be used in such selection systems are concentrations that provide for selective greater proliferation of cells containing the DNA encoding an inhibitor-resistant DHODH relative to at least substantially identical cells that do not contain DNA encoding an inhibitor-resistant DHODH. Such concentrations of inhibitor may be determined empirically by those of skill in the art. The inhibitor may be used at concentrations that provide for proliferation of cells containing DNA encoding an inhibitor-resistant DHODH to the exclusion of proliferation of cells that do not contain DNA encoding an inhibitor-resistant DHODH.

c. Use of the Selection Systems in Selecting Genetically Modified Cells that are Able to Utilize the Salvage Pathway for Pyrimidine Biosynthesis

With certain cells, the use of selection systems that are based on a resistant DHODH as a selectable marker and DHODH inhibitors as selection agents may not provide sufficient inhibition of proliferation of non-transfected cells due to pyrimidine biosynthesis via the salvage pathway. Cells that have sufficient pyrimidine biosynthesis via the salvage pathway that are not sufficiently inhibited in their proliferation by the presence of DHODH inhibitors can be used with these selection systems when an inhibitor(s) of salvage pathway enzymes is included in the selection agent.

Enzymes of the pyrimidine salvage pathway, include, but are not limited to, thymidine kinase, deoxycytidine kinase, uridine kinase and cytidine kinase. The activity of thymidine kinase is unique in that it fluctuates with the cell cycle, rising to peak activity during DNA synthesis making it a beneficial target for antiproliferative drug therapy. Inhibitors of the pyrimidine synthesis salvage pathway include, but are not limited to, direct and indirect inhibitors of salvage pathway enzymes such as AZT [see e.g., Weber et al. (1991) Adv. Enzyme Regul. 31:45-67], cytosinearabinoside (Ara C) [see, e.g., International Patent Application Publication No. WO98/30709], 2′2′-difluorodeoxycytidine (gemcitabine) [see, e.g., Radhey et al. (1992) Oncology Res. 4(11-12):517-522], camptothecin and analogs thereof [see e.g., Voeller et al. (2000) Cancer Chemotherapy and Pharmacology 45(5):409-416], okadaic acid [see e.g., Tsukamoto et al. (1996) Biochem. Pharmacol. 52(6):879-884], organoselenium compounds [see e.g., Tillotson et al. (1994) Carcinogenesis 15(4):607-610], and natriuretic factor and analogs thereof [see, e.g., Cahill et al. (1994) Amer. J. Phys. 266:194-203. Inhibitor-resistant or altered salvage pathway enzymes may also be used as selectable markers alone or in combination with inhibitor-resistant or altered enzymes from the de novo pathway along with inhibitors of the enzymes as selection agents.

C. Selection Systems Using Pyrimidine and Purine Biosynthesis Pathway Enzymes as Selectable Markers

Selection systems using pyrimidine or purine biosynthesis pathway enzymes as selectable markers as described herein can be used independently or in combination. Thus, for example, inhibitor-resistant enzymes of the purine and pyrimidine biosynthesis pathways may be used together as selectable markers in systems which use the corresponding inhibitors of purine and pyrimidine biosynthesis pathway enzymes as selection agents. One advantage of such systems is the ability to conduct multiple selections for transfer of multiple heterologous nucleic acids into host cells. Inhibitor-resistant enzymes of the de novo purine and pyrimidine biosynthesis pathways also may be used as selectable markers in various combinations with de novo pathway inhibitors and purine and pyrimidine salvage pathway inhibitors as selection agents.

D. Transfer and Expression of Nucleic Acids Encoding Inhibitor Resistant Enzymes of the Purine or Pyrimidine Biosynthesis Pathway into Cells

a. Transfer of Nucleic Acids Encoding a Resistant IMPDH or DHODH into Cells

In using the selection systems based in a resistant IMPDH and/or DHODH as a selectable marker to select for cells into which heterologous nucleic acids have been transferred, DNA encoding a resistant IMPDH and/or DHODH and nucleic acid of interest are co-transfected into the cells. The co-transfection may be simultaneous (i.e., cells are exposed to DNA encoding a resistant IMPDH and/or DHODH and the nucleic acid of interest in the same transfection procedure) or sequential in which two or more transfection procedures are conducted for separate transfer of the DNA encoding the resistant IMPDH and/or DHODH and the nucleic acids of interest into the host cells.

DNA encoding a resistant IMPDH or DHODH includes nucleic acid as described herein, obtainable as described herein and/or known to those of skill in the art. Exemplary DNA encoding an inhibitor-resistant IMPDH includes DNA encoding an altered IMPDH which includes DNA encoding an altered human IMPDH type II (see, e.g., SEQ ID NOS. 3, 5, 7, 9 and 11 and any DNA sequence encoding amino acid sequences set forth in SEQ ID NOS. 4, 6, 8, 10 and 12) and DNA encoding an altered mouse IMPDH type II [see, e.g., Lightfoot et al. (1994) Biochim. Biophys. Acta 1217:156-162 and SEQ ID NO. 31 and any DNA sequence encoding the amino acid sequence set forth in SEQ ID NO. 30]. Exemplary DNA encoding a resistant IMPDH also includes DNA encoding T. foetus IMPDH [see, e.g., SEQ ID NO. 13 and Digits et al. (1999) Biochem. 38:15388-97; and any DNA sequence encoding the amino acid sequence set forth in SEQ ID NO. 14]. Exemplary DNA encoding a resistant DHODH includes DNA encoding an altered human DHODH (see, e.g., SEQ ID NO. 23 and any DNA sequence encoding amino acid sequences set forth in SEQ ID NO. 24) and DNA encoding an altered Aspergillus nidulans DHODH [see, e.g., SEQ ID NOS. 25 and 27 and U.S. Pat. No. 5,976,848, Nov. 2, 1999; and any sequence encoding the amino acid sequences set forth in SEQ ID NOS. 26 and 28].

The nucleic acid encoding a resistant IMPDH or DHODH can be operably linked to elements that facilitate expression of the nucleic acid in host cells. Such elements include promoters, enhancers and terminators that are functional in the recipient host cell and are known to those of skill in the art. In a specific embodiment, the promoter is not native to the genes for the resistant IMPDH or DHODH. Exemplary promoters include, but are not limited to the human elongation factor-1α/human T cell leukemia virus (ER-1α/HTLV) hybrid promoter, the human cytomegalovirus (CMV) immediate early 1 promoter-enhancer element, tissue specific promoters, such as MHC class I promoter, the SV40 early promoter [Bernoist and Chambon (1981) Nature 290:304-310], the promoter contained in the 3′ long terminal repeat of Rous sarcoma virus [Yamamoto et al. (1980) Cell 22:787-797], the herpes thymidine kinase promoter [Wagner et al. (1981) Proc. Natl. Acad. Sci. USA 78:1441-1445], the regulatory sequences of the metallothionein gene [Brinster et al. (1982) Nature 296:39-42]; prokaryotic expression vectors such as the β-lactamase promoter [Villa-Kamaroff et al. (1978) Proc. Natl. Acad. Sci. USA 75:3727-3731-1978] or the tac promoter [DeBoer et al. (1983) Proc. Natl. Acad. Sc. USA 80:21-25]; promoter elements from yeast and other fungi such as the Gal4 promoter, the alcohol dehydrogenase promoter, the phosphoglycerol kinase promoter, the alkaline phosphatase promoter, and the following animal transcriptional control regions that exhibit tissue specificity and have been used in transgenic animals: elastase I gene control region which is active in pancreatic acinar cells [Swift et al. (1984) Cell 38:639-646]; Ornitz et al. (1986) Cold Spring Harbor Symp. Quant. Biol. 50:399-409]; MacDonald (9187) Hepatology 7:425-515]; insulin gene control region which is active in pancreatic beta cells [Hanahan et al. (1985) Nature 315:115-122], immunoglobulin gene control region which is active in lymphoid cells [Grosschedl et al. (1984) Cell 38:647-658]; Adams et al. Nature 318:533-538]; Alexander et al. (1987) Mol. Cell Biol. 7:1436-1444], mouse mammary tumor virus control region which is active in testicular, breast, lymphoid and mast cells [Leder et al. (1986) Cell 45:485-495], albumin gene control region which is active in liver (Pinckert et al. (1987) Genes and Devel. 1:268-276], alpha-fetoprotein gene control region which is active in liver [Krumlauf et al. (1985) Mol. Cell. Biol. 5:1639-1648]; Hammer et al. (1987) Science 235:53-58], alpha-1 antitrypsin gene control region which is active in liver [Kelsey et al. (1987) Genes and Devel. 1:161-171], beta globin gene control region which is active in myeloid cells [Mogram et al. (1985) Nature 315:338-340]; Kollias et al. (1986) Cell 46:89-94], myelin basic protein gene control region which is active in oligodendrocyte cells of the brain [Readhead et al. (1987) Cell 48:703-712], myosin light chain-2 gene control region which is active in skeletal muscle [Sani (1985) Nature 314:283-286], and gonadotrophic releasing hormone gene control region which is active in gonadotrophs of the hypothalamus [Mason et al. (1986) Science 234:1372-1378]. Promoters may be elements that facilitate expression of operably linked nucleic acids in eukaryotic, particularly mammalian, cells.

For transfer of nucleic acid encoding a resistant IMPDH and/or DHODH into cells, the nucleic acid may be contained within a vector. Any vector known in the art for transfer and expression of nucleic acids in cells may be used, including plasmids, cosmids and artificial chromosomes. For simultaneous co-transfection, the DNA encoding a resistant IMPDH and/or DHODH and the nucleic acid of interest may be contained on separate vectors or on the same vector in which they can be operably linked to elements that facilitate expression of the nucleic acids in host cells. Multiple sequences, such as a resistant IMPDH and/or DHODH and another nucleic acid of interest, contained on the same vector may be controlled either by a single promoter or by multiple promoters. Exemplary vectors include pMG/HyTk/Δpac (InvivoGen, San Diego, Calif.), which contains a human elongation factor-1α/human T cell leukemia virus (ER-1α/HTLV) hybrid promoter and the immediate-early CMV enhancer/promoter with intron A.

Any methods known to those of skill in the art for the insertion of DNA fragments into a vector may be used to construct expression vectors containing a resistant IMPDH and/or DHODH-encoding DNA and appropriate transcriptional/translational control signals and/or other protein coding sequences. These methods may include in vitro recombinant DNA and synthetic techniques and in vivo recombinants (genetic recombination).

Nucleic acid encoding a resistant IMPDH and/or DHODH may be transferred into the host cells using a variety of procedures which are known in the art. The particular procedure used will depend in part on the host cell. Such procedures include, but are not limited to, direct uptake using calcium phosphate [CaPO₄; see, e.g., Wigler et al. (1979) Proc. Natl. Acad. Sci. U.S.A. 76:1373-1376], polyethylene glycol [PEG]-mediated DNA uptake, electroporation, lipofection [see, e.g., Strauss (1996) Meth. Mol. Biol. 54:307-327], microcell fusion [see, e.g., Lambert (1991) Proc. Natl. Acad. Sci. U.S.A. 88:5907-5911; U.S. Pat. No. 5,396,767, Sawford et al. (1987) Somatic Cell Mol. Genet. 13:279-284; Dhar et al. (1984) Somatic Cell Mol. Genet. 10:547-559; and McNeill-Killary et al. (1995) Meth. Enzymol. 254:133-152], lipid-mediated carrier systems [see, e.g., Teifel et al. (1995) Biotechniques 19:79-80; Albrecht et al. (1996) Ann. Hematol. 72:73-79; Holmen et al. (1995) In Vitro Cell Dev. Biol. Anim. 31:347-351; Remy et al. (1994) Bioconjug. Chem. 5:647-654; Le Bolch et al. (1995) Tetrahedron Lett. 3:6681-6684; Loeffler et al. (1993) Meth. Enzymol. 217:599-618].

Nucleic acid delivery procedures particularly suited for transfer of nucleic acids into hematopoietic cells, including but not limited to, bone marrow cells, stem cells, B cells, peripheral blood T cells and peripheral blood lymphocytes include liposome-mediated delivery [see, e.g., Philip et al. (1993) J. Biol. Chem. 268:16087-16090 and Aksentijevich et al. (1996) Hum. Gen. Ther. 7:1111-1122], adenovirus infection [see, e.g., Ragot et al. (1993) Nature 361:647-650], retroviral transduction [see, e.g., Cochlovius et al. (1998) Cancer Immunol. Immunother. 46:61-66, Bunnell et al. (1995) Proc. Natl. Acad. Sci. U.S.A. 92:7739 and Finer et al. (1994) Blood 83:43], electroporation [see, e.g., Hughes et al. (1996) J. Biol. Chem. 271:5369-5377 and Cron et al. (1997) J. Immunol. Meth. 205:145-150], particle bombardment [see, e.g., Yang et al. (1990) Proc. Natl. Acad. Sci. U.S.A. 87:9568-9572] and direct local injection of the DNA (for in vivo transfer of DNA) [see, e.g., Wolff et al. (1990) Science 247:1465-1468; and Zhu et al. (1993) Science 261:209-211]. DNA delivery procedures that can target the DNA to specific cells include antibody-based methods in which the DNA is incorporated in vesicles [Mannino and Gould-Fogerite (1988) Biotechniques 6:682-690] or retroviruses [Roux et al. (1989) Proc. Natl. Acad. Sci. U.S.A. 86:9079-9083] which are rendered target-specific after bridging with polyclonal or monoclonal antibodies. One such method, referred to as antifection [see, e.g., Hirsch et al. (1993) Transpl. Proc. 25:138-139 and Poncet et al. (1996) Gene Therapy 3:731-738] involves the preparation of an inert antibody-DNA vector which can be delivered to a selected cell population.

Transfer of nucleic acids encoding a resistant IMPDH and/or DHODH into host cells may require stimulation of active cell cycling in the host cell population by activating factors. For example, resting T cells can be resistant to transfection with plasmid DNA. Inducing cell proliferation has been shown to enhance nucleic acid transfection. Furthermore, because rapidly dividing cells, such as proliferating lymphocytes, are predominantly dependent on de novo pathways for purine and pyrimidine nucleotide biosynthesis, specifically pathways involving type II IMPDH and DHODH, such cells are more sensitive to inhibitors of these pathways such as, for example MPA, MMF, and brequinar. Therefore, for certain cells, host cell stimulation may be required for optimal use of selection systems based on resistant IMPDH and/or DHODH enzymes as selectable markers.

Lymphocyte activation is the term generally applied to the series of cellular events that collectively make up the response of lymphocytes, particularly T and B lymphocytes, to antigen. For example, T lymphocyte activation includes the following steps: early signal transduction events, transcriptional activation of a variety of genes, expression of new cell surface molecules, secretion of cytokines (e.g., IL-2), and induction of mitotic activity. Included among the new cell surface molecules are high affinity IL-2 receptors. The binding of T cell growth factors, particularly IL-2 or IL-4, to their receptors initiates a series of events that culminate in mitotic activity. In most CD4+ and some CD8+cells, there is transient production of IL-2 for 1-2 days. During this time, the interaction of IL-2 with the high affinity IL-2 receptor results in T cell growth. Mitotic division of activated T cells results in expansion of clones of cells with the same antigen specificity and thereby augments the immune response to a particular antigen. When T cells are stimulated through the T cell receptor (TCR):CD3 complex, mitotic activity can be detected within 48 to 72 hours in vitro. Depending on the T cell population, this induction of mitosis can reflect cell cycle transition from either GO or G1 to the S phases of the cell cycle.

Antigen-induced activation and differentiation of lymphocytes normally occur in the lymphoid tissues and can be shown in vitro by culturing lymphocytes with activating agents. Possible agents include, but are not limited to antigens recognized by the surface antigen receptors, monoclonal antibodies to TCR-CD3, and mitogens. Mitogens are a class of molecules that can activate T or B cells in a non-antigen-specific manner and induce lymphocyte proliferation. Mitogens include some lectins (e.g., phytohaemagglutinin (PHA), concanavalin A (Con A), pokeweed mitogen (PWM)). Lectins are carbohydrate-binding proteins derived from plants and bacteria. Mitogenic lectins are able to activate lymphocytes by cross linking B cell receptors (BCRs) or TCRs. Mitogen stimulation of lymphocytes in vitro is believed to closely mimic stimulation by specific antigens. PHA and Con A stimulate human and mouse T cells. Lipopolysaccharide (LPS) stimulates mouse B cells, while PWM stimulates both human T and B cells.

Stimulation and activation of lymphocyte host cells in connection with the transfer of nucleic acids encoding a resistant IMPDH and/or DHODH into the cells may be performed prior to, during and/or after delivery of the nucleic acids to host cells using methods known to those skilled in the art. Such methods may include, but are not limited to, contacting host cells with antigens, monoclonal antibodies, cytokines, growth factors, mitogens and combinations thereof. For example, in an exemplary protocol, human peripheral blood mononuclear cells (PBMC) or mouse splenocytes which have been stimulated in vitro with anti-CD3 mAb and IL-2 are cultured for 19.5 to 20 hours in medium containing PHA or Con A, respectively, prior to electroporation-facilitated delivery of heterologous nucleic acids to the cells [see, e.g., Hughes et al. (1996) J. Biol. Chem. 271:5369-5377 and Cron et al. (1997) J. Immunol. Meth. 205:145-150]. In another protocol, mouse B cell blasts are stimulated with LPS (E. coli 055:B5; Sigma Chemial Co., St. Louis, Mo.) for 24 hours and then cocultured (4×10⁶ cells/ml; 6-ml cultures) with virus-producer monolayers in the presence of 50 μg/ml LPS for 24 hours for retroviral-mediated delivery of heterologous nucleic acids to the cells [see, e.g., Agarwal et al. (2000) J. Clin. Invest. 106:245-252 and Zambidis et al. (1997) Mol. Med. 3:212-224]. In a further protocol, lymphocytes are transfected after culture for three days with IL-2 and PHA [see, e.g., Cann et al. (1988) Oncogene 3:123] or Con A [see, e.g., Novak et al. (1992) Mol. Cell. Biol. 12:1515].

b. Growth of Cells into Which Nucleic Acids Encoding a Resistant IMPDH and/or DHODH Have Been Transferred

Host cells that have been subjected to procedures for the delivery of nucleic acid encoding a resistant IMPDH and/or DHODH are cultured under conditions that inhibit proliferation of host cells that do not express the resistant IMPDH and/or DHODH. For example, the cells are grown in the presence of an agent that inhibits the IMPDH and/or DHODH activity of cells that have not taken up the nucleic acid encoding the resistant IMPDH and/or DHODH. Appropriate concentrations of the selection agent to accomplish selective proliferation of cells that express the resistant IMPDH and/or DHODH may be determined empirically.

E. Selection Systems and Use Thereof In Vivo

Selection systems that may be used in in vivo applications are also provided (“in vivo selection systems”). These systems are not only suitable for use in vivo, but may also be used to identify, isolate and/or provide a selective advantage for proliferation of certain cells ex vivo and in vitro. The in vivo selection systems are based in the pairing of a selectable marker with selection agents that provide an environment under which cells containing the selectable marker will exhibit greater proliferation than substantially identical cells, for example T-lymphocytes, that do not contain the selectable marker without substantially affecting a wide variety of cells. Because the selection agent is such that it primarily affects only specific cells (e.g., only those cells containing the selectable marker and/or similar cells that do not contain the marker), and not a wide variety of cells, the selection systems are particularly suitable for use in vivo as well as ex vivo and in vitro.

1. Selection Agents and Conditions

Selection agents and conditions useful in the selection systems described herein are cell specific in that they primarily affect only a limited number of cell types. Thus, although the selection agents and conditions used in the systems herein may have some effects on multiple types of cells, those effects are limited in nature and extent and do not significantly impact the majority of cells in a host organism.

Useful selection agents include, but are not limited to, drugs, substances used as chemotherapeutic agents, biosynthetic enzyme inhibitors, enzymes that degrade and/or inactivate toxic agents, antibiotics, and antibodies. Selection agents include inhibitors of enzymes of the de novo purine and pyrimidine biosynthesis pathways, particularly inhibitors of IMPDH and DHODH, including those described herein.

Inhibitors of enzymes of the purine and pyrimidine biosynthesis pathways are particularly well suited for use as selection agents in in vivo selection systems because of the differential response of cells to such inhibitors. Different cells, as well as the same cells under various conditions, differentially utilize the de novo and salvage pathways for purine and pyrimidine biosynthesis.

For example, although resting lymphocytes are able to meet their pyrimidine nucleotide requirements predominantly through pyrimidine salvage pathways, the metabolic requirement of proliferating lymphocytes, in which pyrimidine pools can increase up to 8-fold relative to resting lymphocytes, necessitates reliance on de novo pyrimidine biosynthesis. Thus, inhibitors of enzymes of the de novo pathway for pyrimidine biosynthesis, e.g., DHODH inhibitors, will more significantly affect proliferating lymphocytes than resting lymphocytes or any cells that are able to utilize pyrimidine salvage pathways to fulfill pyrimidine nucleotide requirements of the cell. Generally, any cells that have de novo pyrimidine biosynthesis pathway enzyme (e.g., DHODH) activity levels that are elevated relative to those of the majority of cells, at least under certain conditions, will be more significantly affected by inhibitors of such enzymes. Such cells include cells that exhibit increased proliferation, e.g., proliferative responses to mitogens, antigens and other induction agents, or that exhibit regenerative, absorptive or excretory activities (e.g., mucosal cells of the ileum, colon crypts in the gastrointestinal tract, cultured Ehrlich ascites tumor cells, and proximal tubule of the kidney cortex).

Similarly, although most cells are able to use both the de novo an salvage pathways for purine production, lymphocytes are not able to synthesize guanine nucleotides via the salvage pathway. Thus, both resting and proliferating lymphocytes rely on the de novo pathway for purine production. However, IMPDH activity and mRNA levels are induced up to 15-fold upon mitogenic or antigenic stimulation of human peripheral blood T lymphocytes. The predominant IMPDH isoform expressed in proliferating lymphocytes is the type II form, in contrast to the type I form that is the dominant isoform expressed in normal resting human lymphocytes. Generally, any cells that have de novo purine biosynthesis pathway enzyme (e.g., IMPDH) activity levels that are elevated relative to those of the majority of cells, at least under certain conditions, or that rely primarily on IMPDH type II for purine biosynthesis, will be more significantly affected by inhibitors of such enzymes. Such cells include cells that exhibit increased proliferation, e.g., proliferative responses to mitogens, antigens and other induction agents, and transformed tumor cells.

The differential responses of cells to inhibitors of enzymes in the purine and pyrimidine production pathways make possible the design of selection systems that can be used in vivo where it is essential that only a specific target population of cells be affected to any significant extent. Exemplary inhibitors of IMPDH include, but are not limited to, MPA, tiazofurin (2-,β-D-ribofuranosylthiazole-4-carboxamide), ribavirin, 5-ethynyl-1-,β-D-ribofuranosylimidazole-4-carboxamide (EICAR) and mizoribine (brenidin). Exemplary DHODH inhibitors useful in these systems include, but are not limited to, cinchoninic acid derivatives such as Brequinar (6-fluoro-2-(2′-fluoro-1,1′-biphenyl-4-yl)-3-methyl-4-quinoline carboxylic acid), naphthoquinone derivatives such as dichloroally lawsone, isoxazole derivatives such as Leflunomide (N-(4-trifluoromethylphenyl)-5-methylisoxazol-4-carboxamide), and other immunosuppressive and antiproliferative compounds such as NSC 665564, redoxal and BNID.

2. Selectable Markers

Selectable markers for use in the in vivo selection systems are nonimmunogenic or minimally immunogenic when used in immunocompetent host organisms and confer properties that permit proliferation of cells containing the marker under conditions that are not favorable for proliferation of cells that do not contain the marker. Suitable selectable markers include, but are not limited to nucleic acids, proteins, enzymes, carbohydrates, lipids, or other agents resistant to or that degrade/or inactivate toxic agents. Selectable markers include inhibitor-resistant enzymes of the de novo purine and pyrimidine biosynthesis pathways, particularly IMPDH and DHODH.

a. Nonimmunogenic or Minimally Immunogenic Markers

Because the markers provided herein are nonimmunogenic or minimally immunogenic, they do not elicit any significant immune responses in immunocompetent hosts that may adversely interfere with the continued presence of heterologous nucleic acid-containing cells in host organisms. Generally, a marker that is a composition found in a particular species of animal will not be immunogenic when used in a host of the same species. Thus, human protein markers will generally not be immunogenic in human hosts. Mutant human protein markers that do not differ significantly from the unmutated protein are also generally not immunogenic. It is also possible that markers that are compositions found in one species of animal may not be immunogenic when used in a host animal of a closely related species. In addition, immunogenic compositions may be altered to reduce or eliminate their immunogenicity in host animals. Reduction or elimination of immunogenicity may be accomplished, for example, by humanization techniques, epitope mapping and alteration techniques, and protein engineering techniques.

Humanization techniques are well known in the art and generally involve grafting or resurfacing techniques in which variable regions that interact with substrate are linked to human framework sequences. Humanization can be further refined by computer modeling to localize the minimum number of donor derived amino acids needed for substrate recognition. These amino acids may then be modeled into a human framework and a gene encoding the resulting sequence can be synthesized. The protein encoded by this synthetic gene should be less immunogenic in a human host.

Epitope mapping is the precise identification of an epitope associated with a function or structure within a protein. For example, a binding domain of a protein may be determined using an array of approaches. Once an epitope is determined, modifications can be made to prevent HLA and/or antibody recognition. One method of epitope mapping involves the digestion of a pure protein into smaller fragments using specific proteases for different time periods, separation of the fragments on SDS-PAGE (ordering the fragments), transfer onto membrane, binding to antibodies or radioactive ligands, and isolation of the smallest peptide either by affinity chromatography or extraction from gels or membrane for peptide sequencing [see, e.g., Glenney et al., J. Mol. Biol. 167:275-293 (1983)]. Another epitope mapping method involves cloning cDNA encoding the protein of interest into an expression vector. The cloned cDNA is truncated using a restriction endonuclease or Bal 31 nuclease for subsequent expression in an appropriate vector. A truncated protein may then be expressed in vitro by a cellular transcription and translation system followed by immunoprecipitation with an antibody or ligand to identify the smallest protein which binds to it. By identification of a segment of the cDNA corresponding to the expression of that protein, a clone is isolated and sequenced to yield information as to the epitope of interest [see, e.g., Lorenzo et al., Eur. J. Biochem. 176:53-60 (1988)]. Site-directed mutagenesis may also be used in epitope mapping. In this method, oligonucleotides are utilized to generate site specific alterations in cDNA encoding a protein of interest, and the mutant cDNA is introduced into cells which lack the protein. The cells may then express the altered protein which may be assayed for function, e.g., ligand binding [see, e.g., Kashles et al. Proc. Natl. Acad. Sci. U.S.A. 85:9576-9571 (1988)]. Epitope mapping may also be performed by restriction digestion of DNA into multiple fragments followed by insertion into an expression vector for the expression and analysis of the function of the resulting protein. [see, e.g., Kamboj, et al. J. Cell Biol. 107:1835-1843 (1988)].

Upon completion of an epitope map, protein engineering techniques may be employed to alter the protein such that the immune system is unable to process it for antigen presentation. Alternatively, agents may be used which inhibit antigen processing and/or presentation.

A selectable marker may be tested for immunogenicity in a particular host organism using a variety of assays as described herein and/or known in the art. For example, CTL assays may be used for antigens to which the organism has previously generated immunity (see, e.g., WO91/02805), and in vitro generation of T-cell response utilizing dendritic cells transduced with the antigen may be used for antigens to which the organism does not have a previously existing response [see Henderson et al. (1996) Cancer Res. 56:3763-3770; Hsu et al. (1995) Natl. Med. 2:52-58]. Standard skin tests, such as those utilized to test allergic reactions, may also be employed to test the immunogenicity of a marker in a particular organism.

b. Properties Conferred by Markers on Cells Containing the Markers

Selectable markers for use in the in vivo selection systems may confer properties on the cells containing them such as maintenance of cell viability, proliferation and function in presence of cytotoxic and/or cytostatic agents. Selectable markers for use in these systems may be inhibitor-resistant IMPDH and/or DHODH enzymes. Such enzymes provide in cells in which they are expressed increased resistance to inhibition of cell proliferation by an inhibitor of an IMPDH and/or DHODH relative to the resistance of substantially similar cells that do not express the inhibitor-resistant enzyme(s). Resistance to inhibition of cell proliferation can be assessed using methods described herein or known in the art.

3. Use of the Selection Systems in vivo

Use of the selection systems in vivo involves the administration of a selection effective amount of a selection agent (IMPDH or DHODH inhibitor), for example, MPA, MMF, brequinar or a pharmaceutically acceptable salt or derivatives thereof. The selection agent may be administered with a pharmaceutically acceptable, non-toxic, excipient, including solid, semi-solid, liquid or aerosol dosage forms.

Administration of the selection agent can be via a variety of modes and formulations for administering compounds to subjects. For example, the selection agent may be administered orally, nasally, intrabronchially, rectally, parenterally, intravascularly, transdermally (including electrotransport), or topically, in the form of a solid, semi-solid, lyophilized powder, or liquid dosage forms, such as, for example, tablets, suppositories, pills, capsules, powders, solutions, suspensions, emulsions, creams, lotions, aerosols, ointments, injectables and gels, preferably in unit dosage forms suitable for simple administration of precise dosages. The compositions typically will include a pharmaceutical carrier or excipient and an active compound (i.e., the selection agent or pharmaceutically acceptable salt or derivative thereof), and, in addition, may include other medicinal agents, pharmaceutical agent carriers, adjuvants and other such substances.

For oral administration, a pharmaceutically acceptable, non-toxic composition may be formed by the incorporation of excipients, such as, for example, pharmaceutical grades of mannitol, lactose, starch, magnesium stearate, sodium saccharine, talcum, cellulose, glucose, gelatin, sucrose and magnesium carbonate. Such compositions may take several forms, such as solutions, suspensions, tablets, pills, capsules, powders and sustained release formulations. The composition may contain, along with the active ingredient, a diluent, such as lactose, sucrose, dicalcium phosphate, a disintegrant, such as starch or derivatives thereof, a lubricant, such as magnesium stearate, and a binder, such as starch, gum acacia, polyvinylpyrrolidone, gelatin, cellulose and derivatives thereof.

Liquid formulations may, for example, be prepared by dissolving, dispersing an active compound (for example, about 0.1% to about 95%, or 0.1% to about 50%, or about 0.5% to about 20%) and optional pharmaceutical adjuvants in a carrier, such as water, saline, aqueous dextrose, glycerol, and ethanol, to thereby form a solution or suspension. Actual methods of preparing such dosage forms are known, or will be apparent, to those skilled in the art; for example, see Remington's Pharmaceutical Sciences, Mack Publishing Company, Easton, Pa., 15th ed., 1975.

For parenteral administration, the selection agent may be mixed with a carrier, such as, for example, an oily ester such as ethyl oleate and isopropyl myristate. Sterile liquid pharmaceutical compositions, solutions or suspensions can be utilized by, for example, intraperitoneal injection, subcutaneous injection, intramuscular injection or intravenously.

Transdermal administration of the selection agent may be conducted through the use of a patch containing the agent and a carrier that is inert to the agent, is non-toxic to the skin and allows delivery of the agent for systemic absorption into the blood stream via the skin. Carriers for transdermal absorption may include pastes, e.g., absorptive powders dispersed in petroleum or hydrophilic petroleum containing the agent with or without a carrier or a matrix containing the agent; creams and ointments, e.g., viscous liquid or semi-solid emulsions, gels and occlusive devices.

Generally, the selection agents are administered to achieve a selection effective amount. The dose required to obtain a selection effective amount may vary depending on the agent, formulation and individual to whom the agent is administered. Possible selection effective amounts of MPA, MMF or a derivative or analog thereof, may include those set forth for methods of using such compositions to treat various conditions, e.g., stenosis, hyperproliferative vascular disease, autoimmune disorders, psoriasis, inflammatory diseases, rheumatoid arthritis, tumors, viruses and allograft rejection, in U.S. Pat. Nos. 5,380,879, 5,444,072 and PCT International Patent Application Nos. PCT/US92/09932 (WO94/12184), PCT/US93/06410 (WO94/01105). Such amounts include a plasma concentration of about 0.3,pM to 10.0 μM, preferably about 1.0 μM. Oral and intravenous doses of MPA or MMF reported as effective include 0.01 mg/kg to about 100 mg/kg of body weight, with ranges of from about 0.1 mg/kg to about 64.3 mg/kg and about 25 mg/kg to about 60 mg/kg being preferable. MMF is administered for preventing allograft rejection in oral dosages of 0.5, 2.0, 3.0, 3.5 and 4.0 grams per day, corresponding to a daily dosage from about 25 mg/kg to about 60 mg/kg, depending upon the subject and the allograft being treated.

Selection effective amounts of inhibitor for in vivo selection may be significantly less than the ranges provided above for the treatment of specific diseases. Therefore, determination of selection effective amounts may also involve in vitro proliferation assays in which varying doses of selection agent are administered to cells in culture and the number of surviving cells is determined in order to calculate the IC⁵⁰ concentration (i.e, the concentration effective for reducing cell proliferation by 50%).

Selection effective amounts may also be based in in vivo animal studies.

F. Use of the Selection Systems in Association with Treating Immune Disorders

Selection systems provided herein which are specific for the selection of cells containing selectable markers as described herein are particularly useful in connection with treatment of immune disorders, such as primary (congenital) immune deficiency, secondary (acquired) immune disorders and autoimmune disorders, that involve transfer of nucleic acids to lymphocytes, bone marrow cells, or other cells beneficial for the treatment of immune disorders. Primary immune deficiency disorders include, but are not limited to, antibody deficiencies, combined immunodeficiencies, complement deficiencies, and phagocytic cell deficiencies including, but not limited to, X-linked Agammaglobulinemia, severe combined immunodeficiency (SCID), adenosine deaminase (ADA) deficiency, C2 deficiency, purine nucleoside phosphorylase deficiency, granulomatous disease, and leukocyte adhesion deficiency. Secondary immune disorders include, but are not limited to, AIDS, and those resulting from malnutrition, neoplasia and infections. Autoimmune disorders include, but are not limited to, Type I diabetes mellitus, inflammatory bowel disease (e.g., Crohn's disease and ulcerative colitis), systemic lupus erythematosus, chronic active hepatitis, multiple sclerosis, Grave's disease, Hashimoto's thyroiditis, Behcet's syndrome, myastenia gravis, Sjogren's syndrome, pernicious anemia, idiopathic adrenal insufficiency and polyglandular autoimmune syndromes Type I and II.

1. Purine and Pyrimidine Biosynthesis Inhibitor-Based Therapies in the Treatment of Immune Disorders

Immunosuppressive therapy is often used to treat immune disorders. Immunosuppressants such as de novo purine and pyrimidine biosynthesis inhibitors dampen the immune response by inhibiting T- and B-cell proliferation. These compounds also inhibit glycosylation of adhesion molecules involved in leucocyte trafficking to endothelial cells, thus restricting amplification of inflammatory injury often associated with autoimmune disorders. By depleting nucleotide pools, these compounds also deplete tetrahydobiopterin, a co-factor for the inducible form of nitric oxide synthase, thereby suppressing tissue damage mediated by peroxynitrite. Treatment for primary immune disorders, such as chronic granulomatous disease, may include transplantation of allogeneic stem cells containing the required functional gene or autologous stem cells transduced with a therapeutic gene. This type of therapy also requires suppression of the immune system which may be accomplished with immunosuppressants such as purine and pyrimidine biosynthesis inhibitors. A typical, therapeutically effective daily dose for the treatment of most immune conditions is on the order of 0.4 to 30 mg/kg of body weight per day [see, e.g., U.S. Pat. No. 4,992,467]. At these dosage levels, further complications such as diminished anti-tumor response and response to infectious agents may result. Therefore, a therapy for immune disorders that permits the continued presence and function of specific immune cells while suppressing further immune associated injury would be of great benefit.

2. Gene Transfer-Based Therapies in the Treatment of Immune Disorders

Gene transfer-based therapies for treatment of primary immune deficiency have focused predominantly on gene replacement as a way to correct an inherited genetic defect. Immune deficiencies targeted for gene transfer-based therapies have generally been those caused by single gene defects such as SCID, familial hypercholesterolemia, cystic fibrosis, Gaucher's disease, and chronic granulomatous disease.

Although many autoimmune disorders and secondary immune deficiencies do not have a strong genetic basis, their treatment may, nevertheless, be improved by gene therapies. Gene therapy strategies for treatment of such immune disorders predominantly focus on the transfer of genes encoding immunomodulatory products that will alter host immune responses in a beneficial manner. Useful gene encoded products include those that modify antigen presentation, T cell responses, and general downstream effector functions, including inflammation and tissue destruction. Useful gene encoded products may also be regenerative growth factors for the repair of damaged target tissue. Specific examples include CTLA-4 which blocks the costimulatory molecules B7-1 and B7-2 [see, e.g., Finck et al., (1994) Science 265:1225-1227], Type 1 cytokines, such as IL-12, which stimulate Th-1 responses, and Type 2 cytokines, such as IL-4 and IL-10, which counteract Th-1 excessive immunity and have anti-inflammatory properties. IL-1 receptor antagonist and soluble forms of IL-1 and TNF receptors are useful gene encoded products which selectively block IL-1 and TNF [see, e.g., Arend (1993) Adv. Immunol. 54:167-227; Arend et al. (1995) Arthritis Rheum. 38:151-160]. These cytokines, among others, provoke the synthesis of free radicals and proteinases that are the ultimate mediators of tissue destruction and dysfunction.

Genes encoding one or more of these immunomodulatory, anti-inflammatory or protective proteins may be transferred to a variety of sites in the body. Genes may be administered systemically, locally or ex vivo into specific cells such as lymphocytes or antigen presenting cells (APCs). Ex vivo genetic modification of autologous lymphocytes is particularly useful in the treatment of autoimmune disorders. Autologous lymphocytes with specificity for the autoantigen can be transduced with a therapeutic transgene and, when infused back into the patient, will express the transgene at the site of autoimmunity. APCs also provide a valuable target for gene transfer. APCs can be genetically modified to eliminate or inhibit autoreactive T lymphocytes by inhibiting their ability to present antigen or by influencing T-cell responses. For example, the transfer of FasL genes to macrophages eliminates autoreactive T lymphocytes via interactions between FasL expressed on the T cells to which the macrophages are presenting [see, e.g., Zhang et al. (1998) Nat. Biotechnol. 16:1045-1049; Zhang et al. (2000) J. Clin. Invest. 105:813-821].

3. Use of Resistant IMPDH and/or DHODH as a Selectable Marker in Selection of Genetically Modified Cells for the Treatment of Immune Disorders

Selection systems provided herein are useful for in vivo selection of cells transduced by local or systemic or ex vivo delivery of genes. In a particular embodiment, cells are transduced by a viral vector encoding an inhibitor-resistant IMPDH and/or DHODH and a therapeutic gene. The therapeutic gene may be a replacement gene, an immunomodulatory gene or anti-inflammatory gene as discussed above. Systemic delivery of vectors encoding a resistant IMPDH and/or DHODH and therapeutic gene may be used for treatment when disease is disseminated or affects organs that are difficult to access. The vector may also be delivered locally to primary sites of disease. Selective pressure is maintained in vivo by the administration of an IMPDH or DHODH inhibitor for which the modified cells are resistant.

The selection systems provided herein are also particularly applicable for use in selection of ex vivo genetically modified T-cells and APCs. For the treatment of autoimmune disorders, antigen specificity and migratory properties of autoreactive T cells provide an endogenous system for regulated delivery of therapeutic transgene products to the site of autoimmune activity. Genetically modified APCs provide a system for inhibiting autoreactive T cells.

In a particular embodiment, specific T-cell lines, such as autoreactive T-cells, may be engineered to produce a resistant IMPDH and/or DHODH which serves as a selectable marker therein. Specific autoreactive T-cells can be prepared from a donor [see, e.g., Meinl et al. (1997) Am. J. Pathol. 150(2):445-453; Vandevyver et al. (1995) Euoropean J. Immunol. 25(4):958-968] and transduced with an inhibitor-resistant IMPDH and/or DHODH containing retroviral vector which confers resistance to a specific IMPDH and/or DHODH inhibitor. Methods for preparing retroviral vectors [see, e.g., U.S. Pat. No. 5,834,256; WO 96/33282] and methods for T cell transduction are well known in the art [see, e.g, WO 96/33282]. Engineered cells containing a selectable marker may also contain one or more other therapeutic genes for treatment of an immune disorder. For example, immunoregulatory sequences may be introduced that inhibit inflammation or regenerative growth factor sequences may be introduced which function to repair damaged target tissue. Antigen-dependent transgene expression may also be obtained by placing the transgene under the control of a specific antigen-inducible promotor. The transfected cells are exposed to an inhibitor of IMPDH and/or DHODH to which the modified cells are resistant. Only T-cells that have incorporated resistant IMPDH- and/or DHODH-encoding DNA, and any additional therapeutic genes, will proliferate. Engineered T-cells are then infused back into the patient via the peripheral blood stream. Resistant T-cell lines can be used to prevent or treat immune disorders while maintaining immunosuppression and in vivo selective pressure. Immunosuppression is achieved by using the appropriate IMPDH or DHODH inhibitor for which the engineered T-cell lines are resistant.

G. Use of the Selection Systems in Association with Bone Marrow Transplantation and Solid Organ Transplantation

Selection systems provided herein may be used in connection with transplantation of cells, tissues, or organs. Transplantation can be autologous (i.e., the patients own tissue is removed, treated, and returned to the individual), allogeneic (i.e., between members of the same species, having allelic variants of certain genes), or xenogeneic (i.e, between members of different species). In a particular embodiment, the selection systems can be used in connection with bone marrow transplant treatment, in particular, bone marrow transplant due to bone marrow deficiency diseases caused by aplastic anemia, leukemia, lymphoma, chemotherapy, and radiation therapy, or due to immunodeficiency, such as congenital neutropenia or severe combined immunodeficiency syndrome. Selection systems may also be used in connection with solid organ or tissue transplantation treatment, including renal transplants due to end-stage renal failure; cardiac transplants due to terminal cardiac failure; lung or heart/lung transplants due to pulmonary hypertension or cystic fibrosis; liver transplants due to cirrhosis, cancer or biliary atresia; cornea transplants due to dystrophy or keratitis; pancreatic transplants due to diabetes; small bowel transplants due to cancer; and skin transplants due to burn injuries.

A1. Associated Diseases and Treatment

Transplant of tissue and/or cells generally results in some form of rejection. Immunosuppression is the major approach to prevention and management of transplant rejection. The predominant obstacle facing successful immunosuppression therapy is lack of specificity of immunosuppressive drugs. This lack of specificity often results in damage to the graft and/or inability of the immunocompromised host to fight opportunistic infectious agents.

a. Host-Versus-Graft Disease

The leading cause of organ transplant loss is due to chronic rejection which manifests as progressive and irreversible damage to the graft from attack due to host immune responses, host-versus-graft disease (HVGD). Graft survival is therefore dependent upon adequate immunosuppression. Use of purine and pyrimidine biosynthesis inhibitors has proven successful in controlling and reversing HVGD by preventing T- and B-cell proliferation [see e.g., WO96/01111; Kuchle et al., (1990) Transplant. Proc. 23:1803-1806; Williams et al., (1994) Transplantation 57:1223-1231]. While suppressing the immune response responsible for acute and chronic HVGD, this treatment can also result in two additional clinical problems for graft recipient. First, the recipient is particularly susceptible to infections, especially by viruses. Infection by cytomegalovirus, a herpes family virus, is particularly common and may be fatal in the immunosuppressed recipient. Second, transplant patients have an increased proclivity to development of certain tumors, commonly B cell lymphomas, squamous cell carcinoma of the skin, and Kaposi's sarcoma. The B cell lymphomas are thought to be the result of infection by Epstein-Barr virus (EBV). The squamous cell carcinomas of the skin are usually associated with papilloma virus and Kaposi's sarcoma is also likely a virally induced malignancy. A therapy that effectively inhibits HVGD while permitting the presence and function of other T cells able to fight infectious agents would therefore be of great advantage in addressing the problem of HVGD while promoting the efficacy of tissue and organ transplantation.

b. Graft-versus-Host Disease

Bone marrow transplantation (BMT) is most often associated with graft-versus-host disease (GVHD) in which the alloreactive T cells within the transplanted healthy bone marrow attack the recipient's (host's) cells as though they were foreign organisms. One treatment for graft-versus-host disease involves immunosuppressants such as de novo purine and pyrimidine biosynthesis inhibitors. Because bone marrow is unable to adequately salvage nucleotides [see, e.g., Xu et al., (1998) J. of Immun. 160:846-853], treatment with such compounds results in myelotoxicity and possible loss of bone marrow graft. Immunosuppression also results in depletion of anti-tumor T-cells inhibiting the patient's natural defence against malignancies.

Another treatment approach taken to minimize or eliminate GVHD has been to deplete donor bone marrow of T cells in an attempt to remove alloreactive T cells [see, e.g., Martin, et al., (1987) Adv. Immunol. 40:379]. T cell depletion from donor marrow has been found to reduce the occurrence of GVHD, however, this approach has also been shown to reduce the success of bone marrow engraftment. Additionally, depletion of T cells from donor marrow used to treat malignancies reduces the anti-tumor activity of donor cells known as graft versus leukemia response (GVL) or graft-versus-myeloma response (GVM) [see, e.g., Goldman, J. M. et al., (1988) Ann. Intern. Med. 108:806-814; Marmont et al., (1991) Blood 78:2120-2130; WO 97/45145].

Treatment of GVHD by immunosuppression and/or depletion of T-cells in donor marrow also results in deficiency of cytotoxic T lymphocytes specific for viruses, such as cytomegalovirus (CMV) or Epstein-Barr virus (EBV), which are important in the pathogenesis of viral disease in immunocompromised transplant recipients. BMT patients may receive a conditioning treatment prior to transplantation that destroys their T-cells, and thus depend on the recovery of virus-specific T-cell immunity by in vivo proliferation of T-cells derived from the donor marrow or T-cells administered separately when using T-cell depleted bone marrow grafts. However, during the first 100 days after non-T-cell depleted allogeneic marrow transplantation, half the recipients are persistently deficient in CD8+ cytotoxic T lymphocytes specific for CMV [see, e.g., Walter et al., (1995) N. Ingl. J Med. 333:1038-1044]. It has also been shown that EBV causes potentially lethal immunoblastic lymphoma in up to 30% of recipients of T-cell depleted bone marrow allografts [see, e.g., Rooney et al., (1998) Blood 92(5):1549-1555; Hege and Roberts, (1996) Current Opin. in Biotech. 7:629-634]. Therefore, the presence of at least some autologous T cells, T cells within the graft or allogeneic T cells administered separately is beneficial for successful engraftment, anti-tumor responses, and for fighting infectious agents. A therapy that effectively inhibits GVHD while permitting the continued presence and function of other T cells either within the graft or administered separately from the graft would therefore be of great advantage in addressing the problem of GVHD while increasing the probability of successful engraftment.

2. Use of Resistant IMPDH and/or DHODH as a Selectable Marker in Selection of Genetically Modified Stem Cells

The selection systems provided herein are particularly suitable for use in selection of genetically modified hematopoietic stem cells. The ability of hematopoietic stem cells to undergo substantial self-renewal as well as the ability to proliferate and differentiate into all of the hematopoietic lineages makes hematopoietic stem cells the target of choice for a number of gene therapy applications. In a particular embodiment of the systems provided herein, hematopoietic stem cells may be engineered to produce an inhibitor-resistant IMPDH and/or DHODH enzyme which serves as a selectable marker therein. Engineered cells containing a selectable marker may also contain other therapeutic genes for treatment of diseases amenable to gene transfer into hematopoietic stem cells. Such diseases may include, but are not limited to, viral infections, malignancies, thalassemia, sickle cell anemia, adenosine deaminase deficiency, recombinase deficiency, recombinase regulatory gene deficiency, Hunter syndrome, Hurler syndrome, and Gaucher's disease. Diseases other than those associated with hematopoietic cells can also be treated by genetic modification of hematopoietic stem cells. Such diseases may be related to the lack of a particular secreted product including, but not limited to, hormones, enzymes, interferons, and growth factors.

Hematopoietic stem cells may be isolated from any known source of stem cells, including bone marrow, mobilized peripheral blood, and umbilical cord blood. Optionally, expansion of the stem cell population and induction into active cell cycling can be performed prior to or following stem cell isolation. Stem cells can be expanded and/or treated to enter active cell cycling in vivo or in vitro by methods well known to those skilled in the art. These methods include, but are not limited to, the administration of cytokines, hydroxyurea, 5-fluorouracil (5-FU), pokeweed mitogen, and anti-CD3 antibodies [see, e.g., Berardi et al. (1995) Science 267:104; Povey et al., (1998) Blood 92:4080-4089; Shafer et al., (1991) Immunology 88:9760-9765; Jaalouk et al., (2000) Human Gene Therapy 11:1837-1849; Spencer et al., (1996) Blood 87:2579-2587; U.S. Pat. No. 5,665,350; U.S. Pat. No. 5,928,638; WO 96/33282]. Inducing stem cell replication has been shown to enhance gene-carrying retroviral integration [see, e.g., U.S. Pat. Nos. 5,665,350; 5,928,638].

Treated or non-treated stem cells can be transduced by recombinant retroviral vectors by means known to those skilled in the art [see, e.g., WO 96/33281; Povey et al., (1998) Blood 92:4080-4089; Shafer et al., (1991) Immunology 88:9760-9765; Jaalouk et al., (2000) Human Gene Therapy 11:1837-1849; Spencer et al., (1996) Blood 87:2579-2587]. While the use of retroviral vectors to mediate gene therapy may be preferred over other vectors for nucleic acid transduction due to their high transduction efficiency, DNA and other types of nucleic acid delivery systems are contemplated herein. The retroviral vectors encode a resistant IMPDH and/or DHODH as a selective marker and, in addition, may direct the expression of one or more therapeutic agents. The transfected cells are exposed to an inhibitor of IMPDH and/or DHODH to which the modified cells are resistant. Only hematopoietic stem cells that have incorporated resistant IMPDH- and/or DHODH-encoding DNA, and any additional therapeutic genes, will proliferate.

For enhancement of engraftment of the engineered stem cells, quiescence may be induced prior to transplantation. Quiescence can be induced, for example, by removing nutrients from the culture medium causing cells to enter the Go cell cycle phase [see, e.g., U.S. Pat. No. 5,665,350]. The quiescent stem cell population is then transplanted into the host. Immunosuppression with the appropriate IMPDH and/or DHODH inhibitor can then be maintained after transplantation of engineered hematopoietic stem cells without myelotoxic effects and without disrupting expression of any therapeutic agent that the stem cells have been engineered to produce.

3. Use of Resistant IMPDH and/or DHODH as a Selectable Marker in Selection of Genetically Modified T-cells The selection systems provided herein are also particularly suitable for use in selection of genetically modified T-cell lines. In a particular embodiment, specific T-cell lines, such as EBV- or CMV-specific cytotoxic T-cells or antitumor T-cells, may be engineered to produce a resistant IMPDH and/or DHODH which serves as a selectable marker therein. Specific cytotoxic T-cell lines and antitumor T-cell lines, such as EBV- or CMV-specific CD8+ and CD4+cytotoxic T lymphocytes or tumor infiltrating T lymphocytes, can be prepared from donor-derived peripheral blood [see, e.g., Rooney et al. (1998) Blood 92(5):1549-1555; Walter et al. (1995) N. Engl. J. Med. 333:1038-1044] and transduced with a retroviral vector carrying DNA encoding an inhibitor-resistant IMPDH and/or DHODH which confers resistance to a specific IMPDH and/or DHODH inhibitor. Methods for preparing retroviral vectors [see, e.g., U.S. Pat. No. 5,834,256; WO 96/33282] and methods for T cell transduction are well known in the art [see, e.g., WO 96/33282]. Such cell-lines can be made for a wide range of opportunistic infectious entities other than EBV and CMV.

Engineered cells containing a selectable marker may also contain one or more other therapeutic genes for treatment of a viral infection, malignancy or other diseases amenable to gene transfer into T-cells. For example, specific sense, antisense or ribozyme sequences may be introduced that interfere with viral infection, replication or tumor growth or gene products may be introduced which serve as decoys by binding essential viral proteins, thereby interfering with the normal viral life cycle and inhibiting replication. The transfected cells are exposed to an inhibitor(s) of an IMPDH and/or DHODH to which the modified cells are resistant. Only T-cells that have incorporated resistant IMPDH- or DHODH-encoding DNA, and any additional therapeutic genes, will proliferate. Engineered T-cells are then infused back into the patient via the peripheral blood stream. Resistant T-cell lines can be used to prevent or treat infection while maintaining immunosuppression required to prevent organ or tissue rejection. Immunosuppression is achieved by using the appropriate IMPDH and/or DHODH inhibitor for which the engineered T-cell lines are resistant.

H. Use of the Selection Systems in Association with Treating Hyperproliferative Vascular Disease

1. Hyperproliferative Vascular Disease

Hyperproliferative vascular disease is a condition often resulting from vascular smooth muscle cell injury and disruption of the integrity of the endothelial lining. Hyperproliferative vascular disease is initiated by the release of paracrine and autocrine growth factors, such as platelet derived growth factor (PDGF), epidermal growth factor, insulin-like growth factor, and transforming growth factor, as well as cytokines in response to vascular injury. The release of growth factors and cytokines stimulates medial smooth muscle cell proliferation and migration into the intima through fenestrae in the internal elastic lamina. In the intima, the proliferating smooth muscle cells form neointimal lesions. T cells and macrophages also migrate into the neointima [see e.g., WO94/01105].

Vascular injury causing hyperproliferative vascular disease can be broadly categorized as being either biologically or mechanically induced. Biologically mediated vascular injury may include injury attributed to autoimmune disorders, alloimmune related disorders, infectious disorders including endotoxins and herpes viruses, such as cytomegalovirus, metabolic disorders such as atherosclerosis, and vascular injury resulting from hypothermia and irradiation. One of the most commonly occurring forms of biologically mediated vascular injury is atherosclerosis. Occlusive coronary atherosclerosis remains one of the leading causes of mortality and morbidity in industrialized counties.

Mechanical injuries leading to hyperproliferative vascular disease result following balloon angioplasty, vascular surgery, transplantation surgery, laser treatment and other similar invasive processes that disrupt vascular integrity. Chronic rejection in the form of hyperproliferative vascular disease, also known as growth factor-mediated graft vascular disease (GVD), and obliterative bronchiolitis (OB) is one of the major factors limiting long-term cardiac and pulmonary allograft survival [see, e.g., Czech et al., (1999) Inflamm. Res. 48(suppl.2):S128-S129].

2. Purine and Pyrimidine Biosynthesis Inhibitor-Based Therapies in the Treatment of Hyperproliferative Vascular Disease

Purine and pyrimidine biosynthesis inhibitors have been found to inhibit the proliferation of vascular smooth muscle cells [see, e.g., Czech et al. (1999) Inflamm. Res. 48(suppL2):S1128-S129 and WO 94/01105]. Generally, the compounds are administered in a therapeutically effective amount, i.e., a dosage sufficient to effect treatment, which will vary depending on the individual and condition being treated. Therapeutically effective amounts of MMF which inhibit cellular proliferative response to vascular injury typically may be in the range of about 0.3 to about 10.0 pM [see, e.g., WO 94/12184]. The amount required to inhibit smooth muscle proliferation will also result in inhibition of T- and B-lymphocyte proliferation [see, e.g., WO 96/01111 and 5,380,879 and 5,444,072]. T- and B-lymphocyte proliferation is beneficial for successful engraftment, anti-tumor responses, for fighting infectious agents, and many other immune responses. A therapy that effectively inhibits smooth muscle cell proliferation while permitting the continued presence and function of T and B lymphocytes would, therefore, be of great advantage in addressing the problem of hyperproliferative vascular disease while promoting the efficacy of any vascular procedure such as, balloon angioplasty, stent, vascular surgery, transplantation surgery, laser treatment, endovascular or surgical procedures.

3. Gene Transfer-Based Therapies in the Treatment of Hyperproliferative Vascular Disease

Gene transfer-based therapies for the treatment of hyperproliferative vascular disease primarily focus on the viral-mediated localized arterial delivery or ex vivo delivery of therapeutic genes that interfere with the local production and action of growth factors, oncogenes and cell regulatory proteins involved with smooth muscle cell growth. Such therapeutic genes may include, but are not limited to, ribozymes, cyclin dependent kinase inhibitors, growth regulator or tumor suppressor genes such as members of the retinoblastoma gene family, and antisense genes such as AT1 B [see, e.g., 5,834,440; Lamphere et al., (2000) J. Mol. Med. 78(8):451-459; Claudio et al., Circulation Res. 85(11):1032-1039; Ouyang et al., (1999) 27(5):381-383]. Indirect inhibition of smooth muscle cell growth may also be achieved by re-endothelialization using hepatocyte growth factor gene [see, e.g., Hayashi et al., (2000) Gene Therapy 7(19): 1664-1671].

4. Use of Resistant IMPDH and/or DHODH as a Selectable Marker in Selection of Genetically Modified Cells

The selection systems provided herein are particularly suitable for use in protecting autologous T-cell lines against inhibitors of nucleoside metabolism used to treat smooth muscle cell proliferation. In a particular embodiment, specific autologous T-cell lines may be engineered to produce a resistant IMPDH and/or DHODH which serves as a selectable marker therein and serves to protect autologous T cell proliferation in the presence of IMPDH and/or DHODH inhibitors.

T-lymphocytes and macrophages also home to the site of vascular injury making it possible to modify these cells to deliver therapeutic gene products to the site of injury. The selection systems provided herein are therefore useful for the selection of genetically modified T cells containing genes that inhibit smooth muscle cell proliferation or other therapeutic genes as discussed for the treatment of immune disorders and transplantation.

The selection systems provided herein are also particularly suitable for use in the in vivo selection of genetically modified cells. In a particular embodiment, smooth muscle cells or cells responsible for the deposition of proteins involved in the formation of extracellular matrix are transduced by a viral vector encoding resistant IMPDH and/or DHODH and a therapeutic gene that interferes with smooth muscle cell growth. For in vivo delivery, a catheter may be used to deliver the viral vector directly to the site of vascular injury [see, e.g., Maillard et al., (1997) Cardiovascular Res. 35(3):536-546] or the viral vector may be delivered systemically. Ex vivo delivery to graft tissue or organs may be accomplished by pressure mediated transfection [see, e.g., Mann et al., (1999) Lancet 354(9189):1493-1498; Poston et al., (1998) J. Thorac. Cardiovasc. Surg. 116:386-396]. Selective pressure is maintained in vivo and smooth muscle cell growth inhibited by the administration of an IMPDH and/or DHODH inhibitor for which the modified cells are resistant.

I. Use of the Selection Systems in Association with Treating Malignancies

Selection systems provided herein may also be used in connection with treatment of malignancies, in particular, malignancies of lymphoreticular origin, such as, for example, lymphomas and leukemias, including B, T and promonocyte cell line malignancies, mycoses fungoides, non-Hodgkins lymphoma, malignancies of Burkitt lymphoma cells and other EBV-transformed B-lymphocytes, lymphomas resulting from EBV infections in allograft recipients, chronic lymphocytic leukemia, acute lymphocytic leukemia and hairy cell leukemia.

1. General Treatment of Malignancies

A wide range of protocols exist for the treatment of malignancies. Such protocols may include any one or more of the following: immune activators, tumor proliferation inhibitors, antibody therapies, adoptive cellular immunotherapy, gene therapy and drug therapy. Immune activators function by improving immune recognition of tumor-specific antigens such that the immune system is stimulated to promote increased lymphocyte proliferation, differentiation, or evolution to higher affinity interactions. The immune system thus stimulated will more effectively inhibit or kill tumor cells. Immune activators can be modulatory molecules, which affect the interaction between lymphocyte and tumor cell, or lymphokines, which act to proliferate, activate or differentiate immune effector cells. Modulatory molecules may include CD3, ICAM-1, ICAM-2, LFA-1, LFA-3, b-2-microglobulin, chaperones, alpha interferon, gamma interferon, B7/BB1, major histocompatibility complex (MHC), T cell receptor proteins, or synthetic equivalents such as T cell receptors with modified recognition sites. Lymphokines may include gamma interferon, tumor necrosis factor, IL-1, IL-2, IL-3, IL-4, IL-5, IL-6, IL-7, IL-8, IL-9, IL-10, IL-11, GM-CSF, CSF-1, and G-CSF.

a. Gene Transfer-Based Therapies in the Treatment of Malignancies

Much effort has been aimed at enhancing the therapeutic efficacy of tumor immunotherapy through the genetic modification of T cells. Two general types of T-cell gene therapy have been pursued. The first involves modification of T cell specificity. Modification of T cell specificity can be achieved, for example, through the cloning and transfer of genes for T cell receptors (TCRs) with defined tumor-antigen specificity into T cell lines or construction and transfer of chimeric TCRs with novel antigen specificity into T cell lines. For example, the α and β TCR genes from a cytotoxic T lymphocyte (CTL) clone recognizing the MART-1 melanoma antigen have been introduced into a T-cell line, resulting in the transfer of melanoma-specific reactivity [see, e.g., Cole et al., (1995) Cancer Res 55:748-752] To broaden the spectrum of tumors amenable to adoptive T-cell therapy, chimeric receptors composed of single-chain antibodies (scFv) directed against tumor-associated antigens fused to a signaling molecule such as the Fc receptor have been constructed and introduced into T cells [see, e.g., Hwu et al. (1993) J. Exp. Med. 178:361-366; Eshhar et al. (1993) Proc. Natl. Acad. Sci. USA 90:720-724; Hwu et al. (1995) Cancer Res. 55:3369-3373; Moritz et al. (1995) Gene Ther. 2:539-546].

The second type of T cell gene therapy involves arming T cell lines with genes which express immune activators and/or tumor proliferation inhibitors. Numerous examples of this type of gene therapy have been studied. For example, tumor-infiltrating lymphocytes (TILs) have been modified with the gene for tumor necrosis factor [see, e.g, Treisman, et al. (1994) Cell Immunol. 156:448-457; Hwu et al. (1993) J. Immunol. 150:4104-4115], a fusion gene containing TNF and the signal peptide from IFN-gamma [see, e.g., Hwu et al. (1993) J. Immunol. 150:4104-4115], the gene for IL-2 [see, e.g., Nakamura, et al., (1994) Cancer Res 54:5757-5760], chimeric receptors composed of the extracellular portion of the erythropoietin receptor and either the IL-2R,β or gamma chain [see e.g., Minamoto et al, (1995) Blood 86:2281-2287], and intracellular signaling molecule fyn [see e.g., Fujita et al., (1994) Jpn. J. Cancer Res. 85:1073-1079].

b. Purine and Pyrimidine Biosynthesis Inhibitor-Based Therapies in the Treatment of Malignancies

Anti-proliferative agents such as purine and pyrimidine biosynthesis pathway inhibitors are widely used chemotherapy agents. Purine and pyrimidine biosynthesis inhibitors have been found to inhibit cellular proliferation and stimulate differentiation in a wide variety of malignancies, in particular, solid tumors, such as, lung, colon, breast and stomach, and malignancies of lymphoreticular origin, such as, lymphomas and leukemias, including B, T and promonocyte cell line malignancies, mycoses fungoides, non-Hodgkins lymphoma, malignancies of Burkitt lymphoma cells and other EBV-transformed B-lymphocytes, lymphomas resulting from EBV infections in allograft recipients, chronic lymphocytic leukemia, acute lymphocytic leukemia and hairy cell leukemia [see, e.g., 6,054,472; 5,380,879; WO98/13047].

Therapeutically effective amounts of purine and pyrimidine biosynthesis inhibitors for the treatment of malignancies are in the range of those used for immunosuppression. An oral dose of 10-200 mg/kg of body weight has been reported as effective in inhibiting proliferation [see e.g., 4,680,299; WO98/13047]. The amount required to treat malignancies will also result in inhibition of T- and B-lymphocyte proliferation [see, e.g., WO 96/01111 and 5,380,879 and 5,444,072]. Inhibition of T- and B-lymphocyte proliferation results in an increased susceptibility to infection and tumor proliferation. Therefore, a therapy that successfully inhibits malignant cellular proliferation while permitting the continued presence and function of the immune system would be advantageous.

2. Use of Resistant IMPDH and/or DHODH as a Selectable Marker in Selection of Genetically Modified T-cells

The selection systems provided herein are particularly suitable for use in protecting autologous T-cell lines against anti-proliferative agents. The selection system are particularly suitable for use in protecting autologous anti-tumor T-cell lines against inhibitors of nucleoside metabolism used to treat malignancies. In a particular embodiment, specific autologous anti-tumor T-cell lines may be engineered to produce a resistant IMPDH or DHODH which serves as a selectable marker therein and serves to protect autologous T cell proliferation in the presence of IMPDH and/or DHODH inhibitors.

The selection systems provided herein are also particularly suitable for use in selection of genetically modified T-cell lines. One or more anti-tumor agents may be selected and a vector construct can be obtained combining the anti tumor agent(s) with a selective marker provided herein. Autologous or allogeneic T-cell lines are transfected with the vector containing nucleic acids encoding the anti-tumor agent of interest and a resistant IMPDH and/or DHODH. Methods for preparing retroviral vectors [see, e.g., U.S. Pat. No. 5,834,256; WO 96/33282] and methods for T cell transduction are known in the art [see, e.g, WO 96/33282]. While the use of retroviral vectors to mediate gene therapy may be preferred over other vectors for nucleic acid transduction due to their high transduction efficiency, DNA and other types of nucleic acid delivery systems are contemplated herein. The transfected cells are exposed to an inhibitor of IMPDH and/or DHODH to which the modified cells are resistant. Only cells that have incorporated foreign nucleic acids into the cell genome in a manner providing for stable expression of the resistant IMPDH- or DHODH-encoding DNA will proliferate. It will be recognized by those trained in the art that other appropriate cell lines such as hematopoietic stem cells or other cells capable of being used as therapeutic vehicles for particular malignancies may also be used.

Engineered T-cells are then infused back into the patient via the peripheral blood stream. Modified T-cell lines can be used to prevent or treat malignancy in combination with anti-proliferative agents without harmful effects to the modified T-cells and expression of the anti-tumor agent encoded therein. Appropriate anti-proliferative agents are the IMPDH or DHODH inhibitors for which the engineered T-cell lines are resistant.

J. Use of the Selection Systems in Association with Treating Viruses

Selection systems provided herein may also be used in connection with treatment of viruses, for example: retroviruses, including human T-leukemia viruses, Types I and II (HTLV-1 and HTLV-2), human immuno deficiency viruses, Types I and II (HIV-1, HIV-2) and, human nasopharyngeal carcinoma virus (NPCV) and in treating Herpes viruses, including EBV, CMV, Herpes Virus Type 6, Herpes Simplex, Types 1 and 2, (HSV-1, HSV-2) and Herpes Zoster.

1. Purine and Pyrimidine Biosynthesis Inhibitor-Based Therapies in the Treatment of Viruses

Purine and pyrimidine biosynthesis inhibitors have been found to inhibit retroviral replication by depleting substrate pools for reverse transcriptase [see, e.g., Chapuis et aL., (2000) Nature Medicine 6(7):762-768]. Such inhibitors have also been shown to potentiate the antiviral activities of reputed anti-viral compounds such as acyclovir, ganciclovir, penciclovir, H2G ((R)-9(4-hydroxy-2-(hydroxymethyl)butyl)guanine), lobucavir, and abacavir against HSV-1, HSV-2, thymidine kinase deficient HSV-1, CMV, EBV, and HIV [see, e.g., Neyts et al., (1999) Transplantation 67(5):760-764; Margolis et al., (1999) J. Acq. Immune Def. Synd. 21(5):362-370]. The combination of antiviral agents and inhibitors of nucleoside metabolism has, therefore, proven to be an important strategy in the treatment of viral infection.

Therapeutically effective amounts of purine and pyrimidine biosynthesis inhibitors for the treatment of viral infections are in the range of those used for immunosuppression. Oral doses of 200 mg/kg of body weight and plasma concentrations of 0.5-23 μM have been reported as effective in potentiating antiviral efficacy [see e.g., Neyts et aL., (1999) Transplantation 67(5):760-764]. The amount required to treat viral infection will also result in inhibition of T- and B-lymphocyte proliferation [see, e.g., WO 96/01111 and 5,380,879 and 5,444,072]. Inhibition of T-lymphocyte proliferation results in deficiency of cytotoxic T lymphocytes specific for viruses which are important in the pathogenesis of viral disease. Therefore, a therapy that effectively inhibits viral replication while permitting the continued presence and function of specific cytotoxic T lymphocytes would be of great advantage in the treatment of viral infections.

2. Gene Transfer-Based Therapies in the Treatment of Viral Infections

Gene transfer therapies for treatment of viral infections have focused on the generation of specific cytotoxic T lymphocytes as well as the introduction of genes into specific T lymphocytes to engineer greater specificity toward a viral pathogen or into T cells and hematopoietic cells to confer intracellular resistance to specific viral infection. For example, CMV-specific CD8+ and CD4+cytotoxic T lymphocyte clones have been used to restore cellular immunity to CMV in allogeneic bone marrow transplant recipients [see, e.g., Walter et al., (1995) 333:1038-1044; Ridell et al., (1992) Science 257:238-241; Rooney et al., (1998) Blood 92(5):1549-1555; Walter et al., (1995) N. Engl. J. Med. 333:1038-1044].

Specific genes may be introduced to T-cell clones to engineer greater specificity toward a viral agent. For example, transfer of the neo^{r} gene into donor-derived EBV-specific T cells has been used to treat EBV [see, e.g., Heslop et al., (1996) Nat. Med. 2:551-555; Rooney et al., (1995) Lancet 345:9-13]. Another example is the engineering of T cells, a human natural killer cell line, and hematopoietic stem cells with antigen-specific chimeric immune receptors for MHC-unrestricted specificity for HIV [see, et al., (Roberts et al., (1994) Blood 84:2878-2889; Tran et al., (1994) J. Immunol. 155:1000-1009; Hege and Roberts (1996) Current Opinion in Biotechnology 7:629-634].

Various strategies for immunizing T cells and hematopoietic stem cells through the introduction of genes that inhibit viral replication have been proposed [see, e.g., Baltimore, (1988) Intracellular Immunization 335:395-396]. Specific gene therapy strategies targeting retroviral replication cycles have been designed, including the introduction of transdominant HIV proteins [see, e.g., Lisziewicz et al. (1995) Gene Ther. 2:218-222; Woffendin et al., (1994) Proc. Natl. Acad. Sci. USA 91:11581-11585], ribozymes [see, e.g., Yu et al., (1995) Proc. Natl. Acad. Sci. USA 92:699-703; Yamada et al., (1994) Gene Ther. 1:38-45; Leavitt et al., (1994) Hum. Gene. Ther. 5:1115-1120; Zhou et al., (1994) Gene 149:33-39], RNA decoys [see, e.g., Bevec et al., (1994) Hum. Gene. Ther. 5:193-201; Lee et al., (1994) J. Virol. 68:8254-8264], and intracellular antibodies [see, e.g., Maciejewski et al., (1995) Nat. Med. 1:667-673; Duan et al. (1994) Proc. Natl. Acad. Sci. USA 91:5015-5019].

3. Use of Resistant IMPDH and/or DHODH as a Selectable Marker in Selection of Genetically Modified Cells for the Treatment of Viral Infections

The selection systems provided herein are particularly suitable for use in selection of genetically modified T-cell or stem cell lines. The selection system are particularly suitable for use in protecting autologous T-cell lines against inhibitors of nucleoside metabolism used to treat viral infections. In a particular embodiment, specific autologous T-cell lines, such as virus-specific cytotoxic T-cells, may be engineered to produce a resistant IMPDH and/or DHODH which serves as a selectable marker therein and serves to protect autologous T cell proliferation in the presence of IMPDH and/or DHODH inhibitors. Specific cytotoxic T-cell lines such as EBV- or CMV-specific CD8+ and CD4+cytotoxic T lymphocytes can be prepared from donor-derived peripheral blood [see, e.g., Rooney et al., (1998) Blood 92(5):1549-1555; Walter et al., (1995) N. Engl. J. Med. 333:1038-1044] and transduced with a retroviral vector containing nucleic acids encoding a resistant IMPDH and/or DHODH which confers resistance to a specific IMPDH and/or DHODH inhibitor. Methods for preparing retroviral vectors [see, e.g., U.S. Pat. No. 5,834,256; WO 96/33282] and methods for T cell transduction are well known in the art [see, e.g, WO 96/33282]. Such cell-lines can be made for a wide range of viral agents. Engineered stem cell lines, which have the ability to proliferate and differentiate into all of the hematopoietic lineages, may be used as a means to produce resistant T cell lines in vivo. DHODH and/or IMPDH inhibitor-resistant T cell clones or stem cells can be delivered back to the patient to provide protection in the presence of an inhibitor.

Autologous or allogeneic T cells or stem cells containing a selectable marker may also contain one or more other therapeutic genes for treatment of a viral infection as discussed above. Genes may be introduced to T cells to engineer greater specificity toward a viral pathogen or into T cells and hematopoietic cells to confer intracellular resistance to specific viral infection. Such genes may include specific sense, antisense or ribozyme sequences that interfere with viral infection or replication or gene products may be introduced which serve as decoys by binding essential viral proteins, thereby interfering with the normal viral life cycle and inhibiting replication.

The transfected cells are exposed to an inhibitor of wild-type enzyme to which the modified cells are resistant. Only T-cells that have incorporated resistant IMPDH- and/or DHODH-encoding DNA, and any additional therapeutic genes, will proliferate. Engineered T-cells are then infused back into the patient via the peripheral blood stream. Resistant T-cell lines can be used to prevent or treat infection while maintaining a treatment protocol which employs inhibitors of nucleoside metabolism with or without additional antiviral agents. Treatment is achieved by using the appropriate IMPDH and/or DHODH inhibitor for which the engineered T-cell lines are resistant.

K. Use of the Selection Systems in Association with the Treatment of Renal Disorders and Diseases

1. Glomerulonephritis

Glomerulonephritis is a condition of inflammation of the glomeruli of the kidneys. IgA nephropathy is the most common form of glomerulonephritis. The kidneys of people affected with IgAN show deposits of IgA-containing immune complexes with proliferation of the glomerular mesangium. The glomerular mesangium contains mesangial cells and extracellular matrix and plays a crucial role in maintaining structure and function of the glomerular capillary tuft. Mesangial cells play an important role in physiologic functions, including the regulation of glomerular filtration, and as a pathogenic factor for proliferative glomerulonephritis. Mesangial cell hyperplasia is a key feature of many glomerular diseases caused by immunological or other mechanisms of injury, including IgAN, membranoproliferative glomerulonephritis or lupus nephritis.

Mesangial cells are located in the pericapillary mesangial space of the glomerular capillary tuft and can be considered as specialized smooth muscle cells. A primary function of glomerular mesangial cells is maintaining structure and function of the glomerular capillary ultrafiltration apparatus. Imbalances in the control of mesangial cell replication appear to play a key role in the pathogenesis of progressive renal failure.

The turnover of mesangial cells in normal adult kidney is very low with a renewal rate of less than 1%. A prominent feature of glomerular inflammatory diseases is mesangial hyperplasia due to elevated proliferation rate or reduced cell loss of mesangial cells. When mesangial cell proliferation is induced without cell loss, for example due to mitogenic stimulation resulting from by products of mesangioproliferative glomerulonephritis can result.

Regulators of mesangial cell growth include a variety of soluble factors, such as, for example, autacoids, growth factors, hormones and cytokines [see, e.g., Haas et al. (1999) J. Nephrol. 12:405-415]. Promitogenic factors include platelet-derived growth factor (PDGF), basic fibroblast growth factor (bFGF), endothelin (ET-1), interleukin-1β(IL-1β) and serotonin. Antimitogenic factors which may inhibit mesangial cell proliferation include heparin, TGF-β, IL-10, IL-4, interferon-y, nitric oxide and adrenomedullin.

2. Purine and Pyrimidine Biosynthesis Inhibitor-Based Therapies in the Treatment of Renal Disorders and Diseases

Mitogen-induced mesangial cell proliferation can be inhibited by mycophenolate mofetil (MMF) in non-toxic, therapeutic concentrations [see, e.g., Hauser et al. (1999) Nephrol. Dial. Transplant. 14:58-63]. Therapeutically relevant blood through levels of MMF are in the low micromolar range corresponding to 1-5 μg/ml (4.3 μg/ml=10 μM) and are reached with a daily dosage of 2-3 g MMF in adult transplant recipients being treated for the prophylaxis of allograft rejection [see, e.g., Sollinger et al. (1992) Transplantation 53:428-432]. The growth inhibitory effect of MMF on mesangial cells appears to be mediated by inhibition of IMPDH.

3. Gene Transfer-Based Therapies in the Treatment of Renal Diseases and Disorders

Gene transfer systems may prove useful in combating a variety of kidney-related diseases and disorders, including kidney transplant rejection and autoimmune, polycystic, malignant and other acute glomerular and chronic interstitial kidney diseases. In vivo and ex vivo approaches to gene transfer to the kidney have been investigated in connection with gene transfer-based therapies in the treatment of renal diseases and disorders. For example, HVJ (a Sendai virus) liposomes, retroviruses, and adenoviruses have been used as in vivo gene transfer vectors whereas tubular epithelial cells and mesangial cells have been used as carrier cells to deliver heterologous nucleic acids into the interstitium and glomerulus.

Mesangial cells can be genetically modified to serve as gene carrier cells. For example, foreign genes can be introduced ex vivo into mesangial cells by viral vectors, DNA carriers, gene gun or other methods known in the art and propagated from isolated glomeruli selected on hygromycin which are then transferred back into the glomeruli via the renal circulation. The expression of the foreign gene product can be detected in isolated glomeruli [see, e.g., Kitamura et al. (1994) J. Clin. Invest. 94:497-505; Kitamura et al. (1994) Exp. Nephrol. 4:56-69; Kitamura (1997) Exp. Nephrol. 5:118-125]. In vivo gene transfer to glomerular cells (possible mesangial cells) may be achieved using an HVJ sendai virus fused to foreign DNA and a nucleoprotein encapsulated in liposomes [see, e.g., Tomita et al. (1992) Biochem. Biophys. Res. Commun. 186:129-134].

4. Use of Resistant IMPDH and/or DHODH as a Selectable Marker in Selection of Genetically Modified Mesangial Cells

Selection systems provided herein can provide a means of facilitating both ex vivo and in vivo gene transfer to the kidney. For example, selection systems provided herein are particularly suitable for use in selection of genetically modified mesangial cells. In a particular application of the systems provided herein, mesangial cells may be engineered to produce an inhibitor-resistant IMPDH and/or DHODH which serves as a selectable marker therein. Mesangial cells are transfected with foreign nucleic acids of interest and DNA encoding a resistant IMPDH and/or DHODH. The transfected cells are exposed to an inhibitor of wild-type IMPDH to which the modified cells are resistant. Only mesangial cells that have incorporated foreign nucleic acids into the cell genome in a manner providing for stable expression of the resistant IMPDH-encoding DNA will proliferate.

The following examples are included for illustrative purposes only and are not intended to limit the scope of the invention.

### EXAMPLE 1

Effect of Hygromycin on Proliferation of Jurkat T Cells and OKT3/rhlL-2 Activated Peripheral Blood Mononuclear Cells

Jurkat T cells (Human CD4+ T cell line from American Type Culture Collection, ATCC Number: TIB-152) were passaged in T cell culture media containing 0, 100, 125, 150, 175, and 200 μg/ml hygromycin. Unless otherwise stated, T cell culture media was prepared by addition of 13.7 ml HEPES (Irvine Scientific catalog No. 9319), 5 ml L-Glutamine (BioWhittaker Catalog No. 17-605C) and 57 ml heat inactivated fetal calf serum (Hyclone catalog No. SH30070.03) to 500 ml RPMI 1640 medium without Phenol Red pH indicator (Irvine Scientific catalog No. 9160).

Five hundred million freshly isolated, healthy donor peripheral blood mononuclear cells (PBMCs) were diluted to a volume of 500 ml with T cell culture media. PBMCs were activated with 15 λ anti-CD3 monoclonal antibody Orthoclone OKT3 (Ortho Biotech Inc.). Ten 50 ml aliquots of PBMC/OKT3 were placed in separate flasks. To each PBMC/OKT3 flask, 50 μL of stock 50 U/μl recombinant human IL-2 (Chiron catalog No. 53905-991-01) was added to stimulate proliferation. OKT3/rhIL-2-activated PBMCs were passaged under the same conditions as described for the Jurkat T cells. The amount of Jurkat T cell and PBMC proliferation was determined by measuring DNA incorporation of ³H-Tdr 8, 10, 12, and 15 days thereafter.

As a hygromycin control, Jurkat T cells were transfected by electroporation with vector pMG (InvivoGen, San Diego, Calif.; see SEQ. ID. NO. 33 for the nucleotide sequence of pMG) containing the hygromycin phosphotransferase gene and cDNA encoding an altered human IMPDH type II enzyme (see EXAMPLE 3 for a description of generation of this vector referred to as IMPDH(T333I/S351Y)/pMG). Vector pMG contains the hygromycin phosphotransferase gene under the control of the immediate-early human cytomegalovirus (hCMV) enhancer/promoter with intron A. Vector pMG also contains two transcriptional units allowing for the coexpression of two heterologous genes from a single vector sequence.

The first transcriptional unit of pMG contains a multiple cloning site for insertion of a gene of interest, the hygromycin phosphotransferase gene (hph) and the immediate-early human cytomegalovirus (hCMV) enhancer/promoter with intron A [see, e.g., Chapman et al. (1991) Nuc. Acids Res. 19:3979-3986] located upstream of hph and the multiple cloning site, which drives the expression of hph and any gene of interest inserted into the multiple cloning site as a polycistronic mRNA. The first transcriptional unit also contains a modified ECMV internal ribosomal entry site (IRES) upstream of the hph gene for ribosomal entry in translation of the hph gene. The IRES is modified by insertion of the constitutive E. coli promoter (EM7) within an intron (IM7) into the end of the IRES. In mammalian cells, the E. coli promoter is treated as an intron and is spliced out of the transcript. A polyadenylation signal from the bovine growth hormone (bGh) gene [see, e.g., Goodwin and Rottman (1992) J. Biol. Chem. 267:16330-16334] and a pause site derived from the 3′ flanking region of the human a2 globin gene [see, e.g., Enriquez-Harris et al. (1991) EMBO J. 10:1833-1842] are located at the end of the first transcription unit. Efficient polyadenylation is facilitated by inserting the flanking sequence of the bGh gene 3′ to the standard AAUAAA hexanucleotide sequence.

The second transcriptional unit of pMG contains another multiple cloning site for insertion of a gene of interest and an EF-1α/HTLV hybrid promoter located upstream of this multiple cloning site, which drives the expression of any gene of interest inserted into the multiple cloning site. The hybrid promoter is a modified human elongation factor 1alpha (EF-1alpha) gene promoter [see, e.g., Kim et al. (1990) Gene 91:217-223] that includes the R segment and part of the U5 sequence (R-U5′) of the human T-cell leukemia virus (HTLV) type I long terminal repeat [see, e.g., Takebe et al. (1988) Mol. Cell. Biol 8:466-472]. The Simian Virus 40 (SV40) late polyadenylation signal [see Carswell and Alwine (1989) Mol. Cell. Biol. 9:4248-4258] is located downstream of the multiple cloning site. Vector pMG contains a synthetic polyadenylation site for the first and second transcriptional units at the end of the transcriptional unit based on the rabbit,β-globin gene and containing the AATAAA hexanucleotide sequence and a GT/T-rich sequence with 22-23 nucleotides between them [see, e.g., Levitt et al. (1989) Genes Dev. 3:1019-1025]. A pause site derived from the C2 complement gene [see Moreira et al. (1995) EMBO J. 14:3809-3819] is also located at the 3′ end of the second transcriptional unit. Vector pMG also contains an ori sequence (ori pMB1) located between the SV40 polyadenylation signal and the synthetic polyadenylation site.

To prepare Jurkat T cell transfectants, a volume of cell suspension yielding 35×10⁶ viable Jurkat T cells was harvested. Of these cells, 30×10⁶ were placed in a 50 ml centrifuge tube and centrifuged at 1,200 rpm for 10 min at room temperature. The remaining 5×10⁶ Jurkat cells were placed in a flask with 10 ml Phenol Red-free T cell media and the flask placed in an incubator for use as an electroporation control. After centrifugation of the 30×10⁶ cells, the cell-free supernatant was decanted and the pellet resuspended in 10 ml of hypoosmolar electroporation buffer (Eppendorf catalog No. 4308070.501). The resulting cell suspension was centrifuged at 1,200 rpm for 8 min at room temperature. The cell-free supernatant was decanted and cells were resuspended in a volume of room temperature hypoosmolar electroporation buffer to yield a cell suspension of 20×10⁶ cells/ml. Vector IMPDH(T3331I/S351Y)/pMG (30 μg) was added to 1.2 ml of the 20×10⁶ cells/ml suspension. Three 0.4 ml aliquots of cell/DNA suspension were placed in 0.2 cm electroporation cuvettes and electroporated using an Eppendorf Multiporator operating at 240 V with a pulse time of 40 μsec. Each sample was electroporated with a single pulse followed by a 10 min room temperature incubation. Cells and cell debris were harvested from the cuvettes with a sterile transfer pipette into a 15 ml conical centrifuge tube. Phenol Red-free T cell media was added to a volume of 15 ml and the suspension centrifuged at 1,200 rpm for 8 min at room temperature. The pellet was resuspended in 10 ml of Phenol Red-free T cell media and added to a flask containing 40 ml of Phenol Red-free T cell media. The flask was placed in an incubator. Control cells were electroporated as described above with plasmid-free buffer. On day one and two after electroporation, 25 ml of cell-free media from the transfected cell suspension and 12.5 ml from the control cell suspension were removed and replaced with equal volumes of T cell media containing Phenol Red pH indicator. The pH indicator was used to assess any need for media changes based on acidity (“yellowness”) in order to maintain a physiologic pH (7.2-7.8) of the cell suspension. On day three, hygromycin (Invivogen catalog No. ant-hg) was added to the cell suspensions at concentrations of 0, 100, 125, 150, 175, and 200 pg/ml. The amount of cell proliferation was measured on day 8, 10, 12 and 15.

The same methodology may be used for generating a PBMC hygromycin control. To prepare PBMC transfectants, 50 ml OKT3/rhlL-2-stimulated PBMC suspension is centrifuged at 1,200 rpm for 10 min at room temperature. The cell-free supernatant is decanted and the pellet resuspended in 10 ml of hypoosmolar electroporation buffer (Eppendorf catalog No. 4308070.501). The resulting cell suspension is centrifuged at 1,200 rpm for 8 min at room temperature. The cell-free supernatant is decanted and cells are resuspended in a volume of room temperature hypoosmolar electroporation buffer to yield a cell suspension of 20×10⁶ cells/ml. The plasmid (200 μg) is added to 8 ml of the 20×10⁶ cells/ml suspension. Twenty 0.4 ml aliquots of cell/DNA suspension are placed in 0.2 cm electroporation cuvettes and electroporated using an Eppendorf Multiporator operating at 250 V with a pulse time of 40 μsec. Each sample is electroporated with a single pulse followed by a 10 min room temperature incubation. Cells and cell debris are harvested from the cuvettes with a sterile transfer pipette into a 15 ml conical centrifuge tube. Hypotonic electroporation buffer is added to a volume of 15 ml and the suspension centrifuged at 1,200 rpm for 8 min at room temperature. The pellet is resuspended in 10 ml of Phenol Red-free T cell media. One milliliter of the resulting suspension is added to each of 10 flasks containing 49 ml of Phenol Red-free T cell media and 50 μl of stock rhIL-2. The flask is placed in an incubator. Control cells are electroporated as described above with plasmid-free buffer. On day 5, 7, 9, 11, and 13 after electroporation, 25 ml of cell-free media from the transfected cell suspension is removed and replaced with equal volumes of T cell media containing Phenol Red pH indicator and 50 μl rhlL-2 for a final concentration of 50 U/ml rhlL-2. On day six, the cell suspension is split 1:2 by removing 25 ml of the cell suspension to a new flask and adding 12.5 μl rhlL-2 to each flask. Hygromycin (0, 100, 125, 150, 175, and 200 μg/ml; InvivoGen catalog No. ant-hg) is added to each flask and the amount of cell proliferation is measured at 3 and 5 days thereafter.

To assess proliferation by ³H-Tdr incorporation, a volume of cell suspension containing 5×10⁶ cells was diluted to 10 ml and incubated at 37° C. and 5% CO₂. On days 8, 10, 12, and 15, volumes of cell suspension yielding 0.6×10⁶ cells were placed in 15 ml conical tubes and centrifuged at 1100 rpm for 8 minutes at room temperature. Supernatant was removed and cells were resuspended in 0.6×10⁶ ml T cell media. Aliquots of 0.1 ml each were dispensed into wells of a 96 well round bottom plate. Six wells were used for each clone. ³H-Tdr (ICN catalog No. 24041) was diluted 1:20 with T cell media. Each well was pulsed by adding 50 μl/well (lpCi ³H-Tdr/well) dilute ³H-Tdr and incubating for 4 hours. Wells were harvested onto filter paper using a PhD Cell Harvester and CPM were counted in a liquid scintillation counter by standard scintillation procedures.

The viability of Jurkat T cells and PBMCs was determined using the Trypan Blue method for counting and calculating the number of live cells. The viability assay was performed by mixing 30 μl of cell suspension with 30 μl of 0.2% trypan blue (Sigma catalog No. T-8154). The loading sites of a hemacytometer were each loaded with 30 μl of the mixture and the chamber charged by capillary action. The hemacytometer was placed on a microscope and viewed under 100× magnification. Viable cells (unstained cells) and dead cells (blue stained cells) were counted in two diagonally opposite large corner squares of the hemacytometer and the total number of viable cells in the suspension and percentage of viable cells in the population were calculated. The total number of viable cells in the suspension was calculated by multiplying the mean number of cells counted per square by the dilution factor multiplied by 10⁴. The percentage of viable cells in the population was calculated by dividing the total number of viable cells per ml by the total number of cells per ml multiplied by 100.

The total number of Jurkat T cells and PBMCs was assessed by the Unopette method. The assay was performed by loading a Unopette (Becton, Dickinson and Co. catalog No. 5854/55) reservoir containing diluent with 50 μl cell suspension and mixing the cells and diluent evenly. Each chamber of a hemacytometer was charged with the Unopette and cells were counted in all nine large squares of two counting chambers. The total number of cells was calculated by adding the mean number of cells per chamber plus 10% of the mean number of cells per chamber and dividing by 10.

Proliferation and viability of the Jurkat T cells was significantly reduced at all concentrations of hygromycin in comparison to the degree of proliferation and number of viable cells in culture in the absence of hygromycin. In contrast, the viability in the presence of hygromycin of Jurkat T cells that had been transfected with the pMG-based vector was similar to that of untransfected Jurkat T cells in the absence of hygromycin. The proliferation of Jurkat T cell transfectants in the presence of hygromycin was significantly less than that of untransfected Jurkat T cells in the absence of hygromycin but was significantly greater than the proliferation of untransfected Jurkat T cells in the presence of hygromycin. Proliferation and viability of the untransfected PBMCs was also significantly reduced by hygromycin at all concentrations.

### EXAMPLE 2

Effect of Mycophenolic Acid on Proliferation of Jurkat T Cells and OKT3/rhIL-2-Activated PBMCs

Jurkat T cells (ATCC Number: TIB-152) were passaged in T cell culture media as described in Example 1 containing 1×10⁻⁷M, 5×10⁻⁷M, 1×10⁻⁶M, 1×10⁻⁵M, 2×10⁻⁵M, 4×10⁻⁵M, 6×10⁻⁵M, 8×10⁻⁵M and 1×10⁻⁴M mycophenolic acid (MPA). The amount of cell proliferation and viability were measured by the methods described in Example 1 at 72, 96, and 144 hours thereafter. OKT3/rhlL-2-activated PBMCs from healthy donors were passaged under the above conditions, and the amount of cell proliferation measured at the same time intervals. As an MPA control, Jurkat cells and OKT3/rhIL-2-activated PBMCs were passaged under the above conditions except without MPA, and the amount of cell proliferation and viability measured at the same time intervals.

Proliferation and viability of the Jurkat T cells was significantly reduced by MPA at all concentrations in comparison to the extent of proliferation and viability of the cells in the absence of MPA. Proliferation and viability of the OKT3/rhIL-2-activated PBMCs was significantly reduced by MPA at all concentrations, except the lowest concentration (i.e., 1×10⁻⁷M), in comparison to the extent of proliferation of the cells in the absence of MPA.

### EXAMPLE 3

Transfection of Jurkat T Cells with DNA Encoding an Altered Human IMPDH Type II

Jurkat T cells (ATCC Number: TIB-152) were transfected with DNA encoding an altered human type II inosine monophosphate dehydrogenase (IMPDH II) and DNA encoding hygromycin phosphotransferase (i.e., the hygromycin resistance gene). The hygromycin resistance gene was included in these transfections as a control for selectability.

Isolation and Site-Directed Mutagenesis of the Human Type II IMPDH cDNA

Human type II IMPDH cDNA was cloned by nucleic acid amplification of cDNA derived from mRNA isolated by standard procedures from healthy human donor blast cells activated with Orthoclone OKT3 and rhlL-2. Blast cell activation was achieved by the method described for PBMCs in Example 1. The forward and reverse primers used for cloning were as follows:

5′-gctatctgcaggccgccaccatggccgactacctgattag (SEQ ID NO. 34)

3′-ctagctctagatcagaaaagccgcttctcatac (SEQ ID NO. 35).

The resulting clones were digested with Xbal and ligated into a pSK (Stratagene, La Jolla, Calif.) plasmid digested with Xbal and Smal.

The resulting ligation product was used as a template for the introduction of four point mutations into the type II IMPDH cDNA sequence as follows with reference to SEQ. ID. NO. 1: C1045T, G1046C, C1099A and A1100T. These alterations in the cDNA sequence yield the following alterations in the amino acid sequence encoded by the cDNA: T3331 and S351Y. The QuickChange mutagenesis kit by Stratagene was used for site-directed mutagenesis essentially as described in the manufacturer's instructions. Primers for introducing the four mutations were as follows:

T3331 For:5′-ggctccatctgcattatccaggaagtgctggc (SEQ ID NO. 36)

T3331 Rev:3′-gccagcacttcctggataatgcagatggagcc (SEQ ID NO. 37)

S351Y For:5′-cagcagtgtacaaggtgtatgagtatgcacggcgcttt (SEQ ID NO. 38)

S351Y Rev:3′-caaagcgccgtgcatactcatacaccttgtacactgctg (SEQ ID NO. 39)

The mutated plasmid DNA was transformed into SCS 101 competent (Dam-) cells and a digest of the product was performed with EcoRV and Xbal to elute the 1.545 kb altered IMPDH II cDNA fragment.

Construction of Plasmid IMPDH(T333I/S351Y)/pMG

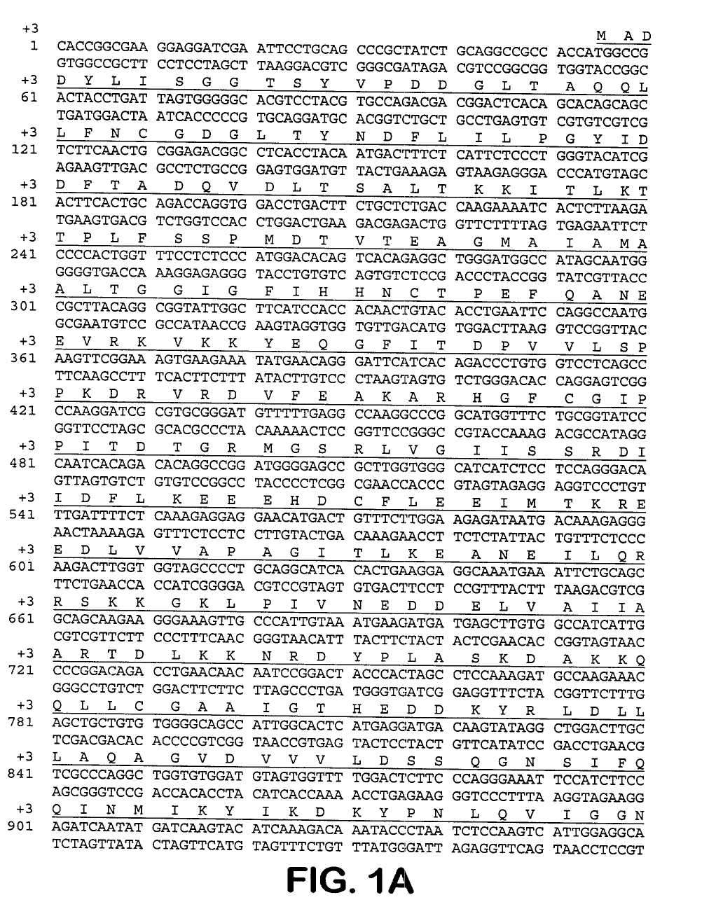

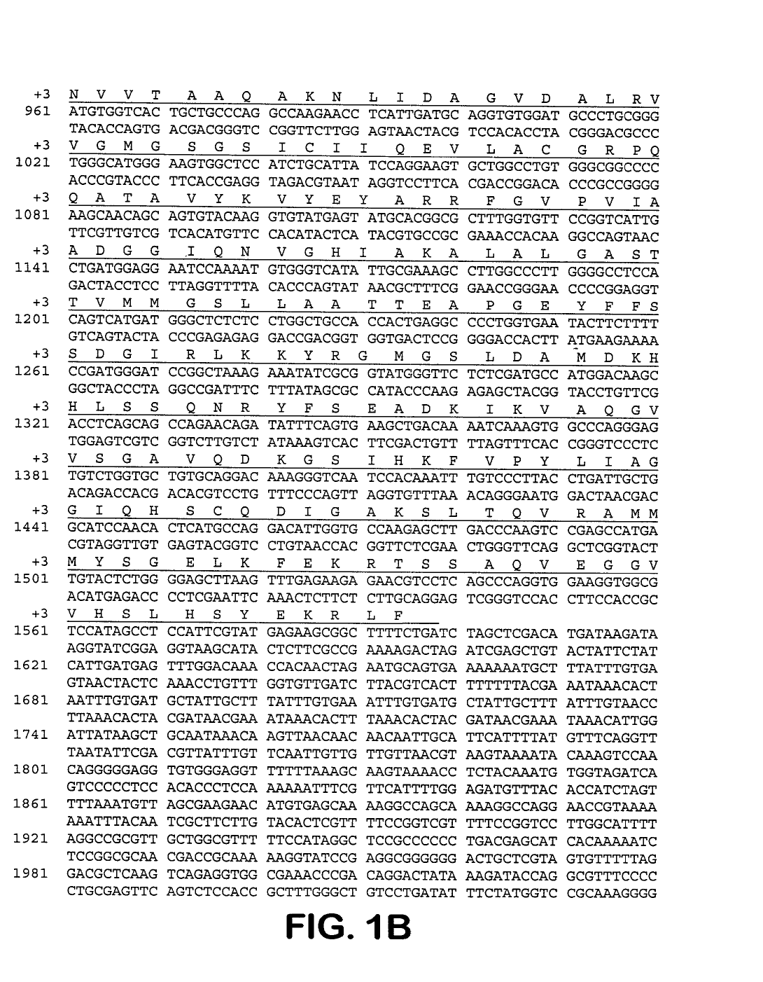

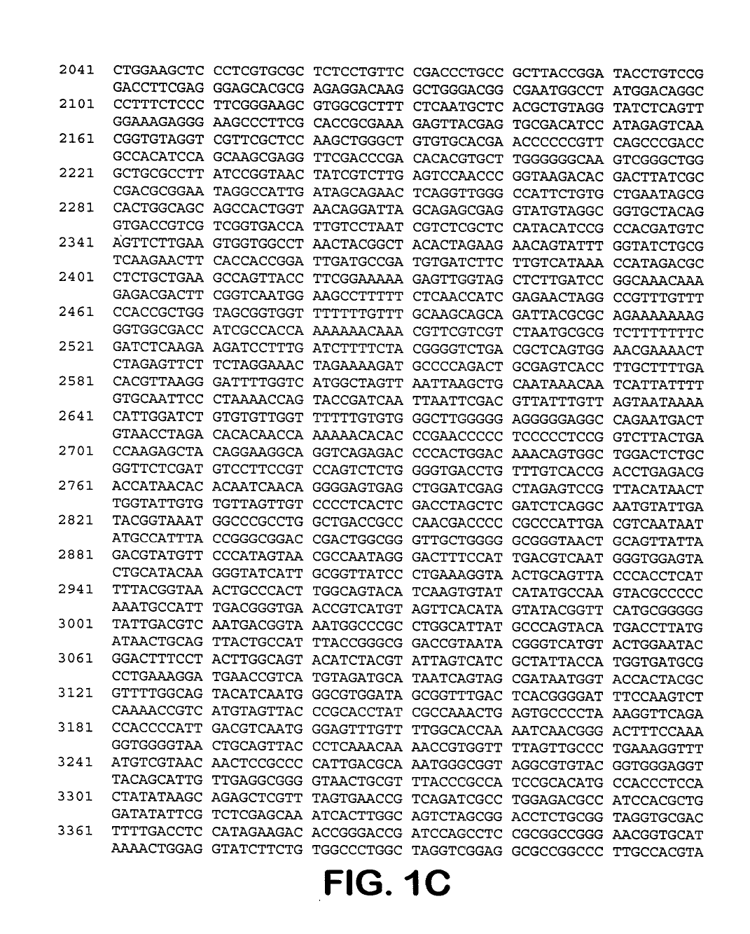

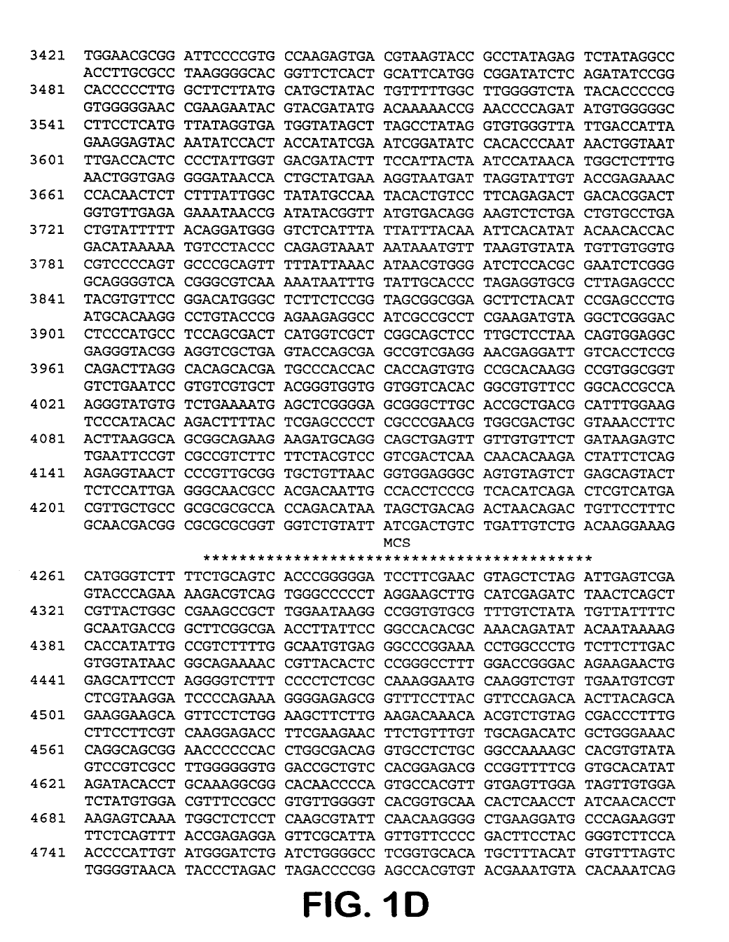

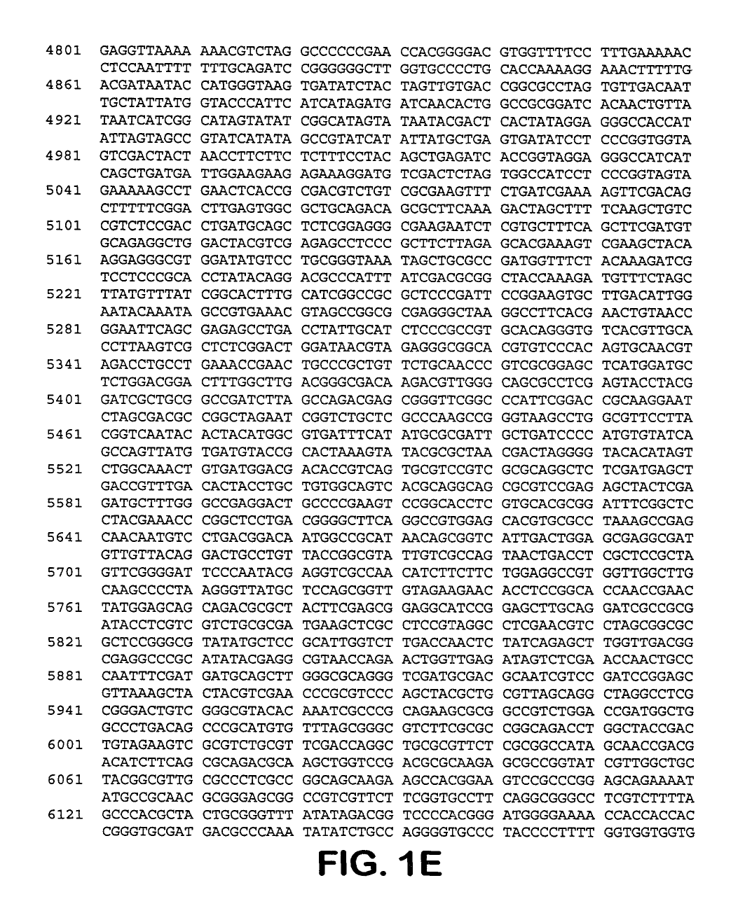

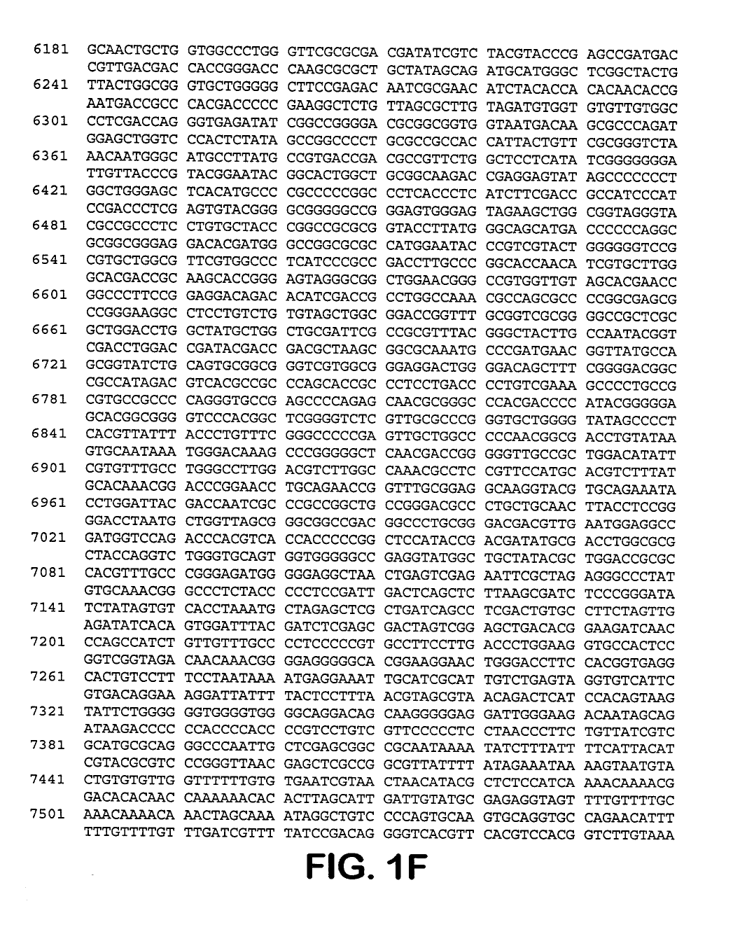

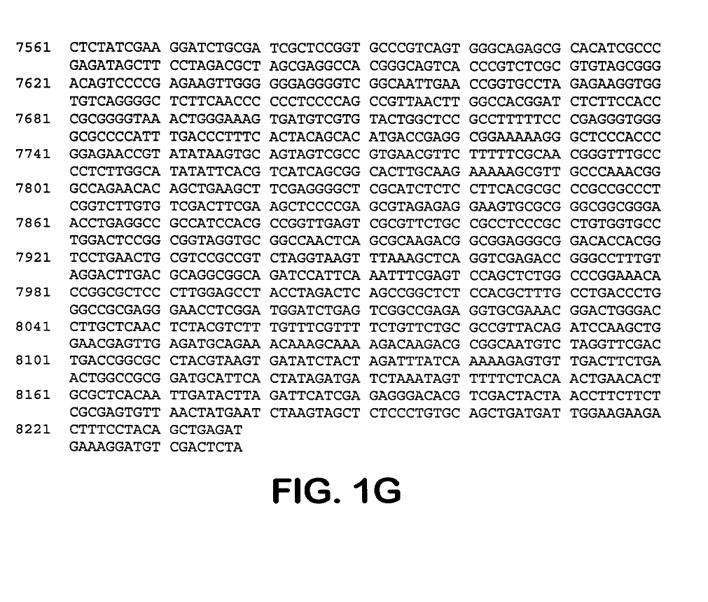

The 1.545 kb altered IMPDH II fragment was subcloned into the multiple cloning site in the second transcriptional unit of the expression vector pMG (InvivoGen, San Diego, Calif.; see SEQ ID NO. 33 for the nucleotide sequence of pMG). Insertion of the altered IMPDH II cDNA into this multiple cloning site of pMG places the cDNA under the control of a modified human Elongation Factor-1αa/Human T cell Leukemia virus hybrid (EF-1α/HTLV) which provides high expression levels in all cell types independent of cell cycle. The promoter is modified to enhance stability of DNA and RNA using the R segment and part of the U5 sequence (R-U5′) of the human T cell leukemia virus (HTLV) Type 1 long terminal repeat [see Takebe et al. (1988) Mol. Cell. Biol. 1:466-472]. The pMG plasmid was cut in the multiple cloning site with Stul and Nhel. The 1.545 kb EcoRV/XbaI fragment encoding the altered IMPDH II was then ligated with the linearized pMG plasmid to generate plasmid IMPDH(T333I/S351Y)/pMG. The nucleotide sequence of such a plasmid is provided in FIG. 1.

Electroporation of Jurkat T Cells with the Plasmid IMPDH(T333I/S351 Y)/pMG

Jurkat T cells (ATCC Number: TIB-152) were grown in T cell culture media in log phase and then subjected to electroporation by the method described in Example 1 in the presence of plasmid DNA vector IMPDH(T333I/S351Y)/pMG as described above. As a control (“mock”), Jurkat T cells were mock-transfected by electroporation with plasmid-free buffer.

Selection of Transfectant Cells by Growth in Hygromycin

Forty-eight hours after transfection, hygromycin was added to a final concentration of 0.8 mg/ml to the culture medium of the transfected and mock-transfected Jurkat T cells. Transfectant cells were grown in T cell culture media containing 0.8 mg/ml hygromycin for 21 days. Twenty-one days after transfection, proliferation and viability of the transfected Jurkat T cells was assessed by the methods described in Example 1. When grown in media containing hygromycin, Jurkat T cells transfected with plasmid IMPDH(T333I/S351Y)/pMG maintained significant proliferation (40-80% of the ³H-Tdr incorporation of the transfected cells in the absence of hygromycin) and viability (80-100% of the viability of transfected cells in the absence of hygromycin). In contrast, the mock-transfected Jurkat T cells did not proliferate when cultured in medium containing hygromycin and there were no viable mock-transfected Jurkat T cells 21 days after transfection.

Growth of Hygromycin-Resistant Transfectant Cells in Mycophenolic Acid

The hygromycin-resistant Jurkat T cells were then transferred to T cell culture media containing 2×10⁻⁷M, 1×10⁻⁶M, 2×10⁻⁶M, 5×10⁻⁶M, 1×10⁻⁵M, 1.5×10⁻⁵M and 2×10⁻⁵M mycophenolic acid but no hygromycin. The effect of mycophenolic acid on the proliferation and viability of the hygromycin-resistant cells was measured at 0, 21, 28, and 36 days after the cells were transferred to medium containing mycophenolic acid. As a control, mock-transfected Jurkat T cells were passaged under the same conditions.

Proliferation and viability of Jurkat T cells was assessed by the methods described in Example 1. The hygromycin-resistant transfectant Jurkat T cells clones continued to proliferate in medium containing mycophenolic acid (ranging, in the presence of 2×10⁻⁶M to 2×10⁻⁷M MPA, from about 20-92% of the maximum proliferation in the absence of MPA). Cells cultured in medium containing higher concentrations of mycophenolic acid were somewhat less viable at day 21 post-transfer to mycophenolic acid-containing medium as compared to day 0. The viability of these cells steadily increased, however, with increasing time post-transfer. Cells cultured in medium containing lower concentrations of mycophenolic acid were more viable at day 21 post-transfer than those cultured in medium containing higher concentrations of mycophenolic acid. The viability of these cells tended to decrease somewhat, however, with increasing time post-transfer.

In contrast, the control (“mock-transfected”) Jurkat T cells did not proliferate when cultured in medium containing mycophenolic acid, and by 36 days after the cells were passaged in the medium containing mycophenolic acid, no viable cells were detected. The extent of proliferation of the mock-transfected control Jurkat T cells in the absence of mycophenolic acid was similar to that of the untransfected host cells in the absence of mycophenolic acid.

Selection of Transfectant Cells by Growth in Mycophenolic Acid

Forty-eight hours after transfection of Jurkat T cells with plasmid IMPDH(T333I/S351Y)/pMG as described above, the transfectant cells were selected and cloned in a limiting dilution, in individual microtiter plate wells, in T cell culture medium containing 3×10⁻⁶ M mycophenolic acid.

Twenty-one days after transfection, the plates were screened for wells with cell growth. Approximately 10% of the transfectant cells demonstrated proliferation at a density of 10⁴ cells per microtiter plate well. Proliferation was assessed by the method described in Example 1.

Cells from cloning wells surviving growth in mycophenolic acid were expanded in T cell culture medium containing 3×10⁻⁶ M mycophenolic acid and assayed for cell proliferation by DNA incorporation of ³H-Tdr in T cell culture medium containing 0, 1×10⁻⁷M, 5×10⁻⁷M, 1×10⁻⁵M, 2×10⁻⁶M, 2.5×10⁻⁶M, 3×10⁻⁶M, 4×10⁻⁶M, 5×10⁻⁶M, 1×10⁻⁵M, 5×10⁻⁵M, and 1×10⁻⁴M mycophenolic acid. ³H-Tdr incorporation in the presence of mycophenolic acid ranged from approximately 28% to 93% of the maximum observed in media without mycophenolic acid, with the greatest inhibition observed at higher mycophenolic acid concentrations. Five of the ten mycophenolic acid-resistant clones maintained greater than 50% maximum ³H-Tdr incorporation (maximum ³H-Tdr incorporation in the absence of MPA) indefinitely at MPA concentrations of 1×10⁻⁶M or less. In contrast, the proliferation of control mock-transfected Jurkat T cells was essentially completely inhibited at lower concentrations of mycophenolic acid, i.e., at concentrations of 2×10⁻⁷ M and greater, ³H-Tdr incorporation was less than 3% of that obtained for media control.

Southern Blot for Detection of IMPDH(T333I/S351Y)/pMG in MPA-Resistant Jurkat Clones

To demonstrate and quantify chromosomally integrated plasmid vector in MPA-resistant Jurkat clones, Southern blot analysis was performed with a DNA probe specific for the hygromycin phosphotransferase hph gene. The hph-specific DNA probe was generated by nucleic acid amplification using hph-specific primers and IMPDH(T333I/S351Y)/pMG plasmid. The hph-specific primers were isolated by electrophoretic separation, purified using Qiaex II gel extraction kit, and labeled with flurorescein-11-dUTP by the random primer method using a Gene Images random prime labeling module (Amersham Life Science, Inc.). Transfectant T cell clones (1×10⁷ cells) were centrifuged at 3,500 rpm for 2 min and genomic DNA was purified using a QIAamp Blood Kit. DNA yield was approximately 50 μg. A restriction digest was performed with BamHI, restriction fragments were separated by gel electrophoresis and Southern blot analysis was performed. All MPA-resistant Jurkat clones studied had a positive Southern blot band indicating the presence of the plasmid vector IMPDH(T3331/S351Y)/pMG.

### EXAMPLE 4

Sequential Selection of Transfectant Jurkat T cells by Growth in Mycophenolic Acid following Growth in Hygromycin

Jurkat T cells transfected with vector IMPDH(T333I/S351Y)/pMG as described in Example 3 and selected by growth in hygromycin as also described in Example 3 were subjected to further selection by subsequent growth in mycophenolic acid.

The hygromycin-resistant transfectant cells were passaged in T cell culture medium containing 3×10⁻⁶M, 4×10⁻⁶M, 5×10⁻⁶M, 6×10⁻⁶M, 7×10⁻⁶M and 8×10⁻⁶M mycophenolic acid. Twenty-eight days after the hygromycin-resistant transfectant Jurkat T cells were passaged in medium containing mycophenolic acid, the viable cells were washed, and a proliferation assay was performed by the method described in Example 1.

The assay was performed by seeding 10⁵ cells per microtiter plate well and culturing the cells in T cell culture medium containing 0, 0.4, 0.8, and 1.0 mg/ml hygromycin or 0, 1×10⁻⁶M, 3×10⁻⁶M, 5×10⁻⁶, 7×10⁻⁶M, 1×10⁻⁵M and 2×10⁻⁵M mycophenolic acid. As a control, mock-transfected Jurkat T cells were cultured under the same conditions.

Proliferation of the selected transfectant Jurkat T cells was observed over a broad range of hygromycin and mycophenolic acid concentrations. Proliferation of the selected transfectant Jurkat T cells was, however, inhibited by hygromycin and mycophenolic acid relative to the proliferation of the transfectant and control cells in media without hygromycin and mycophenolic acid.

Cell proliferation in the presence of hygromycin ranged from approximately 58% to 92% of the maximum observed in media without hygromycin, with the greatest inhibition observed at higher hygromycin concentrations. In contrast, the proliferation of the control Jurkat T cells was completely inhibited by hygromycin at all concentrations.

In the assay to assess the effect of mycophenolic acid on proliferation of transfectant cells, three of the transfectant cell lines were investigated: cells selected in 5×10⁻⁶M, 4×10⁻⁶M and 3×10⁻⁶M mycophenolic acid, respectively. Transfectant cell proliferation in the presence of mycophenolic acid ranged from approximately 62% to 99.7% of the maximum obtained in media without mycophenolic acid, with the greatest inhibition observed at mycophenolic acid concentrations greater than 3×10⁻⁶M. In contrast, the proliferation of the control Jurkat T cells was essentially completely inhibited by mycophenolic acid at all concentrations.

### EXAMPLE 5

Generation and Selection of MPA-resistant PBMCs

Transfectant PBMCs were generated by the electroporation method described in Example 1 using the plasmid IMPDH(T333I/S351Y)/pMG prepared as described in Example 3. PBMC clones were expanded over a repeated fourteen day stimulation cycle in T cell culture media containing 30 ng/ml OKT3 and MPA to a final concentration of 3×10⁻⁶M, 4×10⁻⁶M, 5×10⁻⁵M, 6×10⁻⁶M, 7×10⁻⁶M or 8×10⁻⁶M mycophenolic acid. Beginning on day one of the cycle, IL-2 was added to a final concentration of 50 U/ml and was continually added every 48 hours thereafter. Transfectant cells did not grow in the absence of OKT3 and IL-2.

Southern Blot Analysis of Transfected PMBCs

Genomic DNA was isolated from transfectant PMBCs surviving growth in the presence of MPA and analyzed by Southern blot methods using an hph-specific DNA probe as described in Example 3. The blots revealed the presence of a hybridizing band indicating the presence of vector IMPDH(T333I/S351Y)/pMG in the MPA-resistant clones.

Analysis of Function of Transfected PMBCs

MPA-resistant transfectant PBMCs were evaluated for cytotoxic activity using a CD19 redirected target cell cytolysis assay. The ability of transfectant PMBCs to lyse target cells was determined in a chromium release assay. In this assay, target cells (human erythroleukemia cell line K562, the Daudi human B cell lymphoma line, DHL-6 cells and Allo-LCL cells) were incubated with ⁵¹Cr as follows. The night before performing the assay, the media in a flask of adherent target cells was aspirated and the cells were re-fed with 5 ml media. One mCi ⁵¹Cr was added to the cells and the flasks were swirled to mix completely. The cells were allowed to incubate overnight, and the next morning the media was aspirated and 15 ml of fresh media was added to the cells which were allowed to incubate for 30-60 min.

To label suspension target cells on the day of the assay, 5×10⁶ cells were added to a 50-ml centrifuge tube and centrifuged for 10 min at 1200 rpm and room temperature. The cell-free supernatant was aspirated and the cells were resuspended in 150 μl of FCS (Hyclone # SH30070.03). Fifty microliters of 5 mCi/ml ⁵¹Cr was added to the suspension which was gently vortexed. The cells were allowed to incubate in a 37° C. water bath for 1 hr with gentle vortexing every 15 min. Following the incubation, the tubes of cells were filled with 15 ml assay media (T cell media/2% SDS), vortexed and centrifuged for 10 min at 1200 rpm and room temperature. The cells were resuspended in 10 ml of assay media, vortexed and returned to the water bath for 30-60 min.

Transfected PMBCs (6.5×10⁶ total cells) were transferred into centrifuge tubes and centrifuged in a Beckman GS-6R centrifuge at 1200 RPM at room temperature for 10 min. The cell-free supernatant was aspirated and each pellet was resuspended in 2.6 ml of assay media and diluted. The cells were plated in wells of 96-well V-bottom assay plates.

Adherent target cells were trypsinized and transferred to centrifuge tubes. All target cells were centrifuged for 10 min. at 1200 rpm and room temperature. The supernatant was decanted and the cells were resuspended in 5 ml of assay media. Target cells (100 μl aliquots) were then added to the wells of the plates containing transfected PMBCs to achieve 5000 target cells per well. The plates were centrifuged at 500 rpm for 2 min. and 100 μl of supernate was harvested from each well into tubes. The tubes were placed in a gamma counter and the radioactivity was counted in order to calculate the mean percent chromium release for each target cell. The results of these assays revealed that transfectant PMBCs retained the cytolytic activity of the untransfected control cells.

The results of the experiments described in Examples 1-5 show that Jurkat T cells and PBMCs, genetically modified to express a human type II IMPDH with alterations at amino acids 333 (T->I) and 351 (S->Y) can be selected for by reduced inhibition of proliferation by MPA. The results also show that MPA-resistant Jurkat T cells can proliferate in concentrations of MPA at least two logs higher than tolerated by untransfected Jurkat T cells. Furthermore, transfectant PMBC containing DNA encoding the altered human IMPDH type II not only are able to survive growth in the presence of MPA but retain cytolytic function as well.

MPA-resistant T cells may therefore have the ability to function in vivo in individuals requiring immunosuppression with MPA, such as post-transplant patient, and may permit the use of allogeneic T cells in immunocompetent patients taking MPA, permitting the use of one T cell source for a population of individuals. The results of these experiments further indicate that MPA-resistant T cells would be able to survive peak levels of the drug given orally or, as an IV drip. The serum concentration of MPA can be monitored for effective in vivo selection.

### EXAMPLE 6

In Vitro Determination of the Effect of Inhibitory Compounds on the Response of Human Peripheral Blood Lymphocytes to T- and B-Cell Mitogens

This procedure is a modification of a procedure initially described by Greaves et al. [(1974) Nature 248:698-701]. Human mononuclear cells (PBL) are separated from heparinized whole blood by density gradient centrifugation in Ficoll-Plague (Pharmacia). After washing, 2×10⁵ cells/well are cultured in microtiter plates with RPMI 1640 supplemented with 5% fetal calf serum, penicillin and streptomycin. PHA (Sigma) at 10 μg/ml is then added. Test materials are tested at concentrations between 10⁴ and 10⁸ M, by addition to the culture at time 0. Cultures are set up in quadruplicate and incubated at 37° C. in a humidified atmosphere with 7% CO₂ for 72 hours. A pulse of 0.5 μCi/well of ³H-thymidine is added for the last 6 hours. Cells are collected on glass fiber filters with an automatic harvestor and radioactivity is measured by standard scintillation procedures. The 50% inhibitory concentration (IC₅₀) for mitogenic stimulation is determined graphically.

To evaluate differential effects on T- and B-lymphocytes, different mitogens are used: PWM (Sigma) at 20 μg/ml and Staphylococcus Protein A bound to Sepharose (SPA) (Sigma) 2 mg/ml or 14 μg/ml of Protein A.

### EXAMPLE 7

Determination of the In Vivo Immunosuppressive Activity of Inhibitory Compounds Using the Hemolytic Plaque Forming Cell Assay

This procedure is a modification of the agar plaque technique for recognizing antibody producing cells initially described by Jerne et al. [Cellbound Antibodies, Amos and Kaprowski, eds. (Wistar Institute Press, Philadelphia, 1963) p. 109].

Groups of 5-6 adult C578B1/6 male mice are sensitized with 1×10⁸ sheep red blood cells (SRBC) and simultaneously treated with an oral dosage form of the test material in an aqueous vehicle. Animals in a control group receive the same volume of vehicle. Four days after SRBC inoculation, spleens are dispersed in loose Ten Broeck homogenizers. The number of nucleated cells (WBC) is determined and the spleen cell suspension is mixed with SRBC, guinea pig complements and agar solution at 0.5% concentration. Aliquots of the above mixture (0.1 ml) are dropped on four separate quadrants of a Petri dish and are covered with cover slips. After two hours incubation at 37° C., areas of hemolysis around plaque-forming cells (PFC) are counted with a dissecting microscope. Total WBC/spleen, PFC/spleen and PFC/10⁶ WBC (PPM) are calculated for each mouse spleen. Geometric means of each treatment group are then compared with the vehicle-treated control group.

### EXAMPLE 8

HPLC Assay for Determining IMPDH Activity and Inhibition Thereof

The IMPDH HPLC assay is conducted essentially as described in U.S. Pat. No. 6,054,472. The assay utilizes high pressure liquid chromatography on a C28 column with ion pairing reagents to separate components of the IMPDH-catalyzed, NAD-dependent oxidation of IMP to XMP: IMP, NAD, XMP and NADH. The assay is particularly useful for determining the inhibition profiles of compounds which have significant absorbance in the UV-visible region between 290 and 340 nm.

The reaction mixture typically contains 0.1 M KPi, pH 8.0; 0.1 M KCl, 0.5 mM EDTA, 2 mM DTT, and 0.2 mM each of IMP and NAD. This solution is incubated at 37° C. for 10 minutes. The reaction is started by the addition of enzyme to a final concentration of 20 to 100 nM and is allowed to proceed for 10 minutes. After the allotted time, the reaction is quenched by the addition of mycophenolic acid to a final concentration of 0.01 mM.

The extent of conversion is monitored by HPLC using a Rainin Microsorb ODS column Cl 8-200 of dimensions 4.6×10 mm and a solvent system containing tetrabutylammonium sulfate (5 mM) in 0.1 M KPi, pH 6.0, with a 0-30% methanol gradient over 15 minutes. A similar solvent system has been used previously for the purification of halo-IMP derivatives [Antionio and Wu (1994) Biochemistry 33:1753-1759]. A UV monitor set at 254 nm is used to detect the four components, and the product peaks are integrated to determine the extent of conversion of the substrates.

For the analysis of inhibitors, the compound in question is dissolved in DMSO to a final concentration of 20 nM and added to the initial assay mixture at the desired concentration in a volume of 2-5% (v/v). The reaction is started by the addition of enzyme, and after 10 minutes is quenched as above. After HPLC analysis, the product areas are used to determine the extent of conversion relative to a control assay containing only DMSO and no test compound. IC₅₀ or Kᵢ values are determined from non-linear least squares fitting of conversion vs. concentration curves to the tight-binding equations of Henderson [(1972) Biochem. J. 127:321].

Since modifications will be apparent to those of skill in this art, it is intended that this invention be limited only by the scope of the appended claims.

What is claimed:

1. An isolated altered human inosine monophosphate dehydrogenase (IMPDH) that contains one or more alterations in:
(a) the sequence of amino acids from a position corresponding to amino acid 330 to a position corresponding to amino acid 451 of SEQ. ID. NO. 2;
(b) the sequence of amino acids from a position corresponding to amino acid 330 to a position corresponding to amino acid 441 of SEQ. ID. NO. 2;
(c) the sequence of amino acids from a position corresponding to amino acid 330 to a position corresponding to amino acid 400 of SEQ. ID. NO. 2;
(d) the sequence of amino acids from a position corresponding to amino acid 330 to a position corresponding to amino acid 355 of SEQ. ID. NO. 2; or
(e) the sequence of amino acids from a position corresponding to amino acid 333 to a position corresponding to amino acid 351 of SEQ. ID. NO. 2;
whereby the altered IMPDH is resistant to a purine biosynthesis inhibitor.

2. The isolated IMPDH of claim 1, wherein the purine biosynthesis inhibitor is mycophenolic acid or a derivative, analog or metabolite thereof.

3. The isolated IMPDH of claim 1 containing one or more alterations in the sequence of amino acids from a position corresponding to amino acid 333 to a position corresponding to amino acid 351 of SEQ. ID. NO. 2.

4. The isolated IMPDH of claim 1 containing an amino acid other than threonine at a position corresponding to amino acid 333 of SEQ. ID. NO. 2 or an amino acid other than serine at a position corresponding to amino acid 351 of SEQ. ID. NO. 2 or an amino acid other than threonine at a position corresponding to amino acid 333 of SEQ. ID. NO. 2 and an amino acid other than serine at a position corresponding to amino acid 351 of SEQ. ID. NO. 2.

5. The isolated IMPDH of claim 4, wherein the amino acid other than threonine at a position corresponding to amino acid 333 is isoleucine or a conservative substitution thereof and the amino acid other than serine at a position corresponding to amino acid 351 is tyrosine or a conservative substitution thereof.

6. The IMPDH of claim 1, comprising the sequence of amino acids set forth in SEQ. ID. NO. 4 or the sequence of amino acids set forth in SEQ. ID. NO. 4 containing an alanine at amino acid 190 and a glycine at amino acid 191.

7. An isolated nucleic acid molecule encoding the IMPDH of claim 1.

8. An isolated nucleic acid molecule encoding the IMPDH of claim 2.

9. An isolated nucleic acid molecule encoding the IMPDH of claim 3.

10. An isolated nucleic acid molecule encoding the IMPDH of claim 6.

11. The isolated nucleic acid molecule of claim 10, comprising the sequence of nucleotides 48 to 1589 in SEQ. ID. NO. 3 or the sequence of nucleotides 54-1595 of FIG. 1 or the sequence of nucleotides 48 to 1589 in SEQ. ID. NO. 3 containing the sequence of nucleotides TGCAGG at nucleotides 614-619.

12. The isolated nucleic acid molecule of claim 7, wherein the codon for the amino acid at a position corresponding to amino acid 333 of SEQ. ID. NO. 2 encodes an animo acid other than threonine or the codon for the amino acid at a position corresponding to amino acid 351 of SEQ. ID. NO. 2 encodes an animo acid other than serine or the codon for the amino acid at a position corresponding to amino acid 333 of SEQ. ID. NO. 2 encodes an amino acid other than threonine and the codon for the amino acid at a position corresponding to amino acid 351 of SEQ. ID. NO. 2 encodes an amino acid other than serine.

13. A vector, comprising the nucleic acid molecule of claim 7.

14. A vector, comprising the nucleic acid molecule of claim 10.

15. A cell, comprising heterologous nucleic acid wherein the heterologous nucleic acid comprises the nucleic acid molecule of claim 7.

16. A cell, comprising heterologous nucleic acid wherein the heterologous nucleic acid comprises the nucleic acid molecule of claim 10.

17. An isolated cell, comprising nucleic acid wherein the nucleic acid comprises the nucleic acid molecule of claim 10.

18. The cell of claim 15 that is a T-lymphocyte or a B-lymphocyte.

19. The cell of claim 16 that is a T-lymphocyte or a B-lymphocyte.

20. A non-cancerous human lymphocyte, comprising an altered inosine monophosphate dehydrogenase (IMPDH) that is resistant to a purine biosynthesis inhibitor.

21. The lymphocyte of claim 20, wherein the purine biosynthesis inhibitor is mycophenolic acid or a derivative, metabolite or analog thereof.

22. The lymphocyte of claim 20, wherein the lymphocyte is a T-lymphocyte or B-lymphocyte.

23. A method of providing for selective proliferation, viability or proliferation and viability of a first cell relative to a second cell, comprising:
introducing a nucleic acid encoding an altered mammalian enzyme into the first cell; and
exposing the first and second cells to conditions that inhibit the unaltered mammalian enzyme but to which the altered mammalian enzyme is resistant;
whereby the first cell exhibits greater proliferation, viability or proliferation and viability relative to the second cell.

24. The method of claim 23, wherein the mammalian enzyme is a human enzyme.

25. The method of claim 23, wherein the first and second cells are eukaryotic cells.

26. The method of claim 24, wherein the first and second cells are eukaryotic cells.

27. The method of claim 25, wherein the first and second cells are mammalian cells.

28. The method of claim 26, wherein the first and second cells are mammalian cells.

29. The method of claim 27, wherein the first and second cells are human cells.

30. The method of claim 28, wherein the first and second cells are human cells.

31. A method of providing for selective proliferation, viability or proliferation and viability of a first cell relative to a second cell, comprising:
introducing a nucleic acid encoding an altered mammalian enzyme of a nucleotide biosynthesis pathway into the first cell; and
exposing the first and second cells to conditions that inhibit unaltered mammalian enzyme but to which the altered mammalian enzyme is resistant;
whereby the first cell exhibits greater proliferation, viability or proliferation and viability relative to the second cell.

32. The method of claim 31, wherein the mammalian enzyme is an enzyme of a purine nucleotide biosynthesis pathway.

33. The method of claim 32, wherein the mammalian enzyme is a mammalian inosine monophosphate dehydrogenase (IMPDH).

34. The method of claim 33, wherein the enzyme is a human IMPDH.

35. The method of claim 33, wherein the IMPDH is selected from the group consisting of an IMPDH type I and an IMPDH type II.

36. The method of claim 33, wherein the IMPDH is an IMPDH type II and the conditions comprise exposure to an inhibitor of IMPDH type II.

37. The method of claim 36, wherein the predominant IMPDH isoform expressed in the cells is IMPDH type II.

38. The method of claim 36, wherein the first and/or second cell is selected from the group consisting of lymphocytes, cancer cells, tumor cells, leukemic cells, proliferating cells, and mesangial cells.

39. The method of claim 38, wherein the first and/or second cell is a T-lymphocyte or B-lymphocyte.

40. The method of claim 39, wherein the cell or cells are activated prior to exposure to an inhibitor of IMPDH type II.

41. The method of claim 39, wherein prior to exposure to an inhibitor of IMPDH type II, the cells are exposed to one or more compositions selected from the group consisting of antigens, antibodies, cytokines, growth factors and mitogens.

42. The method of claim 33, wherein the IMPDH is an IMPDH type I and the conditions comprise exposure to an inhibitor of IMPDH type II.

43. The method of claim 42, wherein the predominant IMPDH isoform expressed in the cells is IMPDH type II.

44. The method of claim 33, wherein the IMPDH is an IMPDH type I and the conditions comprise exposure to an inhibitor of IMPDH type I.

45. The method of claim 44, wherein the predominant IMPDH isoform expressed in the cells is IMPDH type I.

46. The method of claim 33, wherein the IMPDH is an IMPDH type I and the conditions comprise exposure to an inhibitor of IMPDH type I and type II or to an inhibitor of IMPDH type I and an inhibitor of IMPDH type II.

47. The method of claim 31, wherein the nucleic acid introduced into the first cell comprises nucleic acid encoding an altered IMPDH type I and nucleic acid encoding an altered IMPDH type II and the conditions comprise exposure to an inhibitor of IMPDH type I and type II or to an inhibitor of IMPDH type I and an inhibitor of IMPDH type II.

48. The method of claim 33, wherein the IMPDH is an IMPDH type II and the conditions comprise exposure to an inhibitor of IMPDH type I.

49. The method of claim 48, wherein the predominant IMPDH isoform expressed in the cells is IMPDH type I.

50. The method of claim 33, wherein the conditions comprise exposure to one or more inhibitors of IMPDH and an inhibitor of a purine salvage pathway enzyme.

51. The method of claim 33, wherein the conditions comprise exposure to mycophenolic acid or a derivative, analog or metabolite thereof.

52. The method of claim 33, wherein the conditions comprise exposure to mycophenolate mofetil.

53. The method of claim 33, further comprising introducing heterologous nucleic acid into the first cell in addition to the nucleic acid encoding an altered inosine monophosphate dehydrogenase enzyme.

54. The method of claim 33, wherein the nucleic acid encoding an altered IMPDH is introduced into the first cell in vitro.

55. The method of claim 33, wherein the nucleic acid encoding an altered IMPDH is introduced into the first cell in vivo in an organism.

56. The method of claim 55, wherein the altered IMPDH is minimally immunogenic in the organism.

57. The method of claim 33, wherein the nucleic acid encoding an altered IMPDH is introduced into the first cell ex vivo and further comprising transfer of the first cell containing the nucleic acid encoding an altered IMPDH or its progeny into an organism.

58. The method of claim 57, wherein the altered IMPDH is minimally immunogenic in the organism.

59. The method of claim 56, wherein the organism is a mammal.

60. The method of claim 59, wherein the organism is a human.

61. The method of claim 58, wherein the organism is a mammal.

62. The method of claim 61, wherein the organism is a human.

63. The method of claim 33, wherein the conditions additionally do not substantially affect cells that are not similar or identical to the first cell into which the nucleic acid encoding an altered IMPDH is introduced.

64. The method of claim 63, wherein the first cell is exposed to the conditions in vivo.

65. The method of claim 33, wherein the nucleic acid encodes the amino acid sequence set forth in SEQ. ID. NO. 32.

66. The method of claim 33, wherein the nucleic acid comprises the sequence of nucleotides 67 to 1611 in SEQ. ID. NO. 31.

67. The method of claim 33, wherein the nucleic acid encodes the amino acid sequence set forth in SEQ. ID. NO. 30 except that the codon for amino acid 333 encodes an animo acid other than threonine.

68. The method of claim 33, wherein the nucleic acid encodes the amino acid sequence set forth in SEQ. ID. NO. 30 except that the codon for amino acid 351 encodes an animo acid other than serine.

69. The method of claim 33, wherein the nucleic acid encodes the amino acid sequence set forth in SEQ. ID. NO. 30 except that the codon for amino acid 333 encodes an amino acid other than threonine and the codon for amino acid 351 encodes an animo acid other than serine.

70. The method of claim 34, wherein the nucleic acid encodes the amino acid sequence set forth in SEQ. ID. NO. 2 except that the codon for amino acid 333 encodes an animo acid other than threonine.

71. The method of claim 34, wherein the nucleic acid encodes the amino acid sequence set forth in SEQ. ID. NO. 2 except that the codon for amino acid 351 encodes an animo acid other than serine.

72. The method of claim 34, wherein the nucleic acid encodes the amino acid sequence set forth in SEQ. ID. NO. 2 except that the codon for amino acid 333 encodes an amino acid other than threonine and the codon for amino acid 351 encodes an animo acid other than serine.

73. The method of claim 34, wherein the nucleic acid encodes the amino acid sequence set forth in SEQ. ID. NO. 4 or the amino acid sequence set forth in SEQ. ID. NO. 4 containing an alanine at amino acid 190 and a glycine at amino acid 191.

74. The method of claim 34, wherein the nucleic acid comprises the sequence of nucleotides 48 to 1589 in SEQ. ID. NO. 3 or the sequence of nucleotides 48 to 1589 in SEQ. ID. NO. 3 containing the sequence of nucleotides TGCAGG at nucleotides 614-619 or the sequence of nucleotides 54-1595 of FIG. 1.

75. The method of claim 34, wherein the nucleic acid encodes the amino acid sequence set forth in SEQ. ID. NO. 2, except that the codon for amino acid 277 encodes an amino acid other than glutamine.

76. The method of claim 34, wherein the nucleic acid encodes the amino acid sequence set forth in SEQ. ID. NO. 2, except that the codon for amino acid 462 encodes an amino acid other than alanine.

77. The method of claim 34, wherein the nucleic acid encodes the amino acid sequence set forth in SEQ. ID. NO. 2, except that the codon for amino acid 277 encodes an amino acid other than glutamine and the codon for amino acid 462 encodes an amino acid other than alanine.

78. The method of claim 34, wherein the nucleic acid encodes the amino acid sequence set forth in SEQ. ID. NO. 2, except that the codon for amino acid 456 encodes an amino acid other than phenylalanine and the codon for amino acid 470 encodes an amino acid other than aspartic acid.

79. The method of claim 34, wherein the nucleic acid encodes the amino acid sequence set forth in any of SEQ. ID. NO. 6, SEQ. ID. NO. 8, SEQ. ID. NO. 10 and SEQ. ID. NO. 12.

80. The method of claim 34, wherein the nucleic acid comprises the sequence of nucleotides 48 to 1589 in any of SEQ. ID. NO. 5, SEQ. ID. NO. 7, SEQ. ID. NO. 9 and SEQ. ID. NO. 11.

81. The method of claim 33, wherein the first cell is a lymphocyte.

82. The method of claim 34, wherein the first cell is a human T-lymphocyte or B-lymphocyte.

83. The method of claim 32, wherein the mammalian enzyme is selected from the group consisting of ribose phosphate pyrophosphokinase, amidophosphoribosyltransferase, glycinamide ribonucleotide (GAR) synthetase, GAR transformylase, formylglycinamidine ribonucleotide (FGAM) synthetase, aminoimidazole ribonucleotide (AIR) synthetase, AIR carboxylase, aminoimidazolesuccinocarboxamide ribonucleotide (SAICAR) synthetase, adenylosuccinate synthase, adenylosuccinate lyase, aminoimidazolecarboxamide ribonucleotide (AICAR) transformylase, inosine monophosphate (IMP) cyclohydrolase and GMP synthase.

84. The method of claim 83, wherein the enzyme is a human enzyme.

85. The method of claim 33, wherein the conditions comprise exposure to one or more compositions selected from the group consisting of ribavirin, tiazofurin, 5-ethynyl-1-β-D-ribofuranosylimidazole-4-carboxamide, mizoribine, selanazole-4-carboxamide adenine dinucleotide, pyridazines and VX-497.

86. The method of claim 31, wherein the mammalian enzyme is an enzyme of a pyrimidine nucleotide biosynthesis pathway.

87. The method of claim 86, wherein the mammalian enzyme is a mammalian dihydroorotate dehydrogenase (DHODH) enzyme.

88. The method of claim 87, wherein the enzyme is a human DHODH.

89. The method of claim 87, wherein the first and/or second cell is selected from the group consisting of lymphocytes, cancer cells, tumor cells, leukemic cells, proliferating cells, and mesangial cells.

90. The method of claim 89, wherein the first and/or second cell is a T-lymphocyte or B-lymphocyte.

91. The method of claim 87, wherein the conditions comprise exposure to an inhibitor of DHODH.

92. The method of claim 90, wherein the conditions comprise exposure to an inhibitor of DHODH and the cells are activated prior to exposure to an inhibitor.

93. The method of claim 90, wherein the conditions comprise exposure to an inhibitor of DHODH, and, prior to exposure to an inhibitor of DHODH, the cells are exposed to one or more compositions selected from the group consisting of antigens, antibodies, cytokines, growth factors and mitogens.

94. The method of claim 87, wherein the conditions comprise exposure to one or more inhibitors of DHODH and an inhibitor of a pyrimidine salvage pathway enzyme.

95. The method of claim 87, wherein the conditions comprise exposure to cinchoninic acid or a derivative, analog or metabolite thereof.

96. The method of claim 87, wherein the conditions comprise exposure to one or more compositions selected from the group consisting of quinolone carboxylic acids, naphthoquinones, isoxazoles, phenoxyquinolines, redoxal and derivatives, analogs or metabolites of any of the foregoing compositions.

97. The method of claim 87, wherein the conditions comprise exposure to one or more compositions selected from the group consisting of 6-fluoro-2-(2′-fluoro-1,1′-biphenyl-4-yl)-3-methyl-4-quinoline carboxylic acid, dichloroally lawsone, lawsone, lapachol, atovaquone, N-(4-trifluoromethylphenyl)-5-methylisoxazol-4-carboxamide and (8-chloro-4-(2-chloro-4-fluoro-phenoxy)quinoline).

98. The method of claim 87, further comprising introducing heterologous nucleic acid into the first cell in addition to the nucleic acid encoding an altered DHODH.

99. The method of claim 87, wherein the nucleic acid encoding an altered DHODH is introduced into the first cell in vitro.

100. The method of claim 87, wherein the nucleic acid encoding an altered DHODH is introduced into the first cell in vivo in an organism.

101. The method of claim 100, wherein the altered DHODH is minimally immunogenic in the organism.

102. The method of claim 87, wherein the nucleic acid encoding an altered DHODH is introduced into the first cell ex vivo and further comprising transfer of the first cell containing the nucleic acid encoding an altered DHODH or its progeny into an organism.

103. The method of claim 102, wherein the altered DHODH is minimally immunogenic in the organism.

104. The method of claim 101, wherein the organism is a mammal.

105. The method of claim 104, wherein the organism is a human.

106. The method of claim 103, wherein the organism is a mammal.

107. The method of claim 106, wherein the organism is a human.

108. The method of claim 87, wherein the conditions do not substantially affect cells that are not similar or identical to the first cell into which the nucleic acid encoding an altered DHODH is introduced.

109. The method of claim 108, wherein the first cell is exposed to the conditions in vivo.

110. The method of claim 87, wherein the nucleic acid encodes the amino acid sequence set forth in SEQ. ID. NO. 24.

111. The method of claim 87, wherein the nucleic acid comprises the sequence of nucleotides 4 to 1101 in SEQ. ID. NO. 23.

112. The method of claim 87, wherein the nucleic acid encodes the amino acid sequence set forth in SEQ. ID. NO. 22 except that the codon for amino acid 26 encodes an animo acid other than histidine.

113. The method of claim 87, wherein the nucleic acid encodes the amino acid sequence set forth in SEQ. ID. NO. 20 except that the codon for amino acid 56 encodes an animo acid other than histidine.

114. The method of claim 87, wherein the nucleic acid encodes the amino acid sequence set forth in SEQ. ID. NO. 22 except that the codon for amino acid 105 encodes an amino acid other than valine.

115. The method of claim 114, wherein the codon for amino acid 105 encodes a glutamic acid residue.

116. The method of claim 87, wherein the nucleic acid encodes the amino acid sequence set forth in SEQ. ID. NO. 20 except that the codon for amino acid 134 encodes an amino acid other than valine.

117. The method of claim 116, wherein the codon for amino acid 134 encodes a glutamic acid residue.

118. A method of providing for selective proliferation, viability or proliferation and viability of a first cell relative to a substantially identical or similar type second cell in vivo comprising, introducing a nucleic acid molecule into the first cell; and exposing the first and second cells in vivo to conditions that inhibit the proliferation, reduce the viability or inhibit the proliferation and reduce the viability of the substantially identical or similar type second cell, whereby the first cell exhibits greater proliferation, viability or proliferation and viability relative to the second cell,
wherein:
the nucleic acid molecule confers resistance to the conditions on the first cell; and
the conditions primarily directly affect only cells similar to the first cell that do not contain the nucleic acid.

119. The method of claim 118, wherein the nucleic acid encodes the amino acid sequence set forth in SEQ. ID. NO. 4 or the amino acid sequence set forth in SEQ. ID. NO. 4 containing an alanine at amino acid 190 and a glycine at amino acid 191.

120. The method of claim 118, wherein the nucleic acid comprises the sequence of nucleotides 48 to 1589 in SEQ. ID. NO. 3 or the sequence of nucleotides 48 to 1589 of SEQ. ID. NO. 3 containing TGCAGG at nucleotides 614-619 or the sequence of nucleotides 54-1595 of FIG. 1.

121. The method of claim 118, wherein the nucleic acid encodes the amino acid sequence set forth in any of SEQ. ID. NO. 6, SEQ. ID. NO. 8, SEQ. ID. NO. 10 and SEQ. ID. NO. 12.

122. The method of claim 118, wherein the nucleic acid comprises the sequence of nucleotides 48 to 1589 in any of SEQ. ID. NO. 5, SEQ. ID. NO. 7, SEQ. ID. NO. 9 and SEQ. ID. NO. 11.

123. The method of claim 118, wherein the nucleic acid encodes the amino acid sequence set forth in SEQ. ID. NO. 24.

124. The method of claim 118, wherein the nucleic acid comprises the sequence of nucleotides 4-1101 in SEQ. ID. NO. 23.

125. The method of claim 118, wherein the first cell is a T-lymphocyte or B-lymphocyte.

126. The method of claim 125, wherein the lymphocyte is a human lymphocyte.

127. The method of claim 118, wherein the condition comprises addition of mycophenolic acid or a derivative, metabolite or analog thereof.

128. The method of claim 118, wherein the condition comprises addition of mycophenolate mofetil.

129. The method of claim 118, further comprising introducing heterologous nucleic acid into the first cell in addition to the nucleic acid molecule that confers resistance to the conditions.

130. The method of claim 118, wherein the nucleic acid molecule that confers resistance to the conditions is introduced into the first cell in vivo in an organism.

131. The method of claim 118, wherein the nucleic acid molecule that confers resistance to the conditions is introduced into the first cell ex vivo and further comprising transfer of the first cell containing the nucleic acid into an organism.

132. The method of claim 118, wherein the nucleic acid that confers resistance to the conditions encodes a protein that is minimally immunogenic in vivo.

133. The method of claim 130, wherein the organism is a mammal.

134. The method of claim 133, wherein the organism is a human.

135. The method of claim 131, wherein the organism is a mammal.

136. The method of claim 135, wherein the organism is a human.

137. A method for transferring a heterologous nucleic acid molecule into an organism, comprising introducing nucleic acids comprising the heterologous nucleic acid molecule and a marker nucleic acid molecule into a first cell in the organism and exposing the organism to conditions that inhibit the proliferation, reduce viability or inhibit proliferation and reduce viability of a substantially identical or similar type cell that does not contain the marker nucleic acid, wherein:
the marker nucleic acid molecule confers resistance to the conditions on the first cell; and
the conditions primarily directly affect only cells similar to the first cell that do not contain the marker nucleic acid.

138. A method for transferring a heterologous nucleic acid molecule into an organism, comprising introducing nucleic acids comprising the heterologous nucleic acid molecule and a marker nucleic acid molecule into a first cell; introducing the first cell into the organism; and exposing the organism to conditions that inhibit the proliferation, reduce viability or inhibit proliferation and reduce viability of a substantially identical or similar type cell that does not contain the marker nucleic acid, wherein:
the marker nucleic acid molecule confers resistance to the conditions on the first cell; and
the conditions primarily directly affect only cells similar to the first cell that do not contain the marker nucleic acid.

139. The method of claim 137, wherein the heterologous nucleic acid molecule encodes a therapeutic product.

140. The method of claim 138, wherein the heterologous nucleic acid molecule encodes a therapeutic product.

141. A method of providing a selective advantage for proliferation of a first cell relative to a second cell, comprising introducing a nucleic acid molecule encoding an altered inosine monophosphate dehydrogenase (IMPDH) into the first cell; wherein
the altered IMPDH is resistant to an inhibitor of purine biosynthesis; and
the first cell is a mammalian cell.

142. The method of claim 141, wherein the nucleic acid molecule encodes the sequence of amino acids set forth in SEQ. ID. NO. 4 or the sequence of amino acids set forth in SEQ. ID. NO. 4 containing an alanine at amino acid 190 and a glycine at amino acid 191.

143. The method of claim 141, wherein the nucleic acid molecule comprises the sequence of nucleotides 89 to 1589 in SEQ. ID. NO. 3 or the sequence of nucleotides 48 to 1589 of SEQ. ID. NO. 3 containing TGCAGG at nucleotides 614-619 or the sequence of nucleotides 54-1595 of FIG. 1.

144. A method of providing a selective advantage for proliferation of a first cell relative to a second cell, comprising introducing a nucleic acid molecule encoding an altered dihydroorotate dehydrogenase (DHODH) into the first cell; wherein
the altered DHODH is resistant to an inhibitor of purine biosynthesis; and
the first cell is a mammalian cell.

145. The method of claim 137, wherein the heterologous nucleic acid molecule encodes a product that alters the organism's immune responses and the conditions comprise administering to the organism an immunosuppressive agent.

146. The method of claim 145, wherein the product is an immunomodulatory, anti-inflammatory or protective protein.

147. The method of claim 145, wherein the immunosuppressive agent comprises an inhibitor of one or more enzymes of one or more nucleotide biosynthetic pathways.

148. The method of claim 145, wherein the organism has an immune disorder.

149. The method of claim 145, wherein the first cell is a lymphocyte.

150. The method of claim 137, wherein the heterologous nucleic acid molecule encodes a product that alters the organism's immune responses and the conditions comprise administering to the organism an immunosuppressive agent.

151. The method of claim 150, wherein the product is an immunomodulatory, anti-inflammatory or protective protein.

152. The method of claim 150, wherein the immunosuppressive agent comprises an inhibitor of one or more enzymes of one or more nucleotide biosynthetic pathways.

153. The method of claim 150, wherein the organism has an immune disorder.

154. The method of claim 150, wherein the first cell is a lymphocyte.

155. The method of claim 118, wherein the conditions comprise administering to the organism an immunosuppressive agent.

156. The method of claim 118, wherein the first cell is a lymphocyte.

157. The method of claim 155, wherein the immunosuppressive agent comprises an inhibitor of one or more enzymes of one or more nucleotide biosynthetic pathways.

158. The method of claim 130, wherein the organism has undergone a bone marrow or solid organ transplantation.

159. The method of claim 130, wherein the organism has undergone a bone marrow or solid organ transplantation.

160. A method of providing for selective proliferation, viability or proliferation and viability of a first cell relative to a second cell, comprising:
introducing a nucleic acid encoding a resistant enzyme into the first cell; and
exposing the first and second cells to conditions that inhibit a sensitive form of the enzyme but to which the resistant enzyme is resistant;
wherein the second cell contains a sensitive form of the enzyme but does not contain the resistant enzyme; and
whereby the first cell exhibits greater proliferation, viability or proliferation and viability relative to the second cell.

161. The method of claim 160, wherein the first and/or second cell is a mammalian cell.

162. The method of claim 161, wherein the resistant enzyme is resistant to an inhibitor of an enzyme of a mammalian nucleotide biosynthesis pathway.

163. The method of claim 162, wherein the resistant enzyme is from a species other than a mammalian species.

164. The method of claim 163, wherein the resistant enzyme is resistant to mycophenolic acid or an analog, derivative or metabolite thereof.

165. The method of claim 164, wherein the resistant enzyme is a Tritrichomonas foetus inosine monophosphate dehydrogenase (IMPDH).
